(19) United States
(12) Patent Application Publication
(10) Pub. No.: US 2024/0316304 A1
(43) Pub. Date: Sep. 26, 2024

(54) HEADGEAR FOR A PATIENT INTERFACE

(71) Applicant: Fisher & Paykel Healthcare Limited, Auckland (NZ)

(72) Inventors: Timothy James HITCHCOCK, Auckland (NZ); Matthew James PEDERSEN, Auckland (NZ); Sanjay Parag PATEL, Auckland (NZ); Paul Mathew FREESTONE, Auckland (NZ); Jeroen HAMMER, Auckland (NZ); Andrew Chun Mon FAN, Auckland (NZ); Bruce Michael WALLS, Auckland (NZ); Chris Onin Limpin HIPOLITO, Auckland (NZ); Mark Arvind MCLAREN, Auckland (NZ); Christopher Jaye Norman AMADIO, Auckland (NZ)

(21) Appl. No.: 18/558,356

(22) PCT Filed: May 19, 2022
(86) PCT No.: PCT/IB2022/054656

§ 371 Date: Oct. 31, 2023

Related U.S. Application Data
(60) Provisional application No. 63/201,938, filed on May 19, 2021.
(60) Provisional application No. 63/280,844, filed on Nov. 18, 2021.

(51) Int. Cl. A61M 16/06

(52) U.S. Cl. CPC A61M 16/0683; A61M 16/0605; A61M 2205/0216; A61M 2207/10; A61M 2209/088; A61M 2210/0618; A61M 2210/0625

(57) ABSTRACT

Disclosed is a headgear for a patient interface, where the headgear includes a first and second lapped panels. The lapped panels define a lapped region in which the first and second panels respectively overlap and underlap each other. The lapped panels also define a non-lapped region in which the first panel is not lapped. At the lapped region, adjacent surfaces of the respective lapped panels are fused together.

### TECHNICAL FIELD

The present invention relates to headgear for a patent interface.

### BACKGROUND

Respiratory interfaces or masks are used to provide respiratory gas or gases, such as air in CPAP therapy, including in for example VPAP and BiPAP systems, or NIV, or high flow rate therapy, for example.

A respiratory interface may comprise a nasal, oral, or full face, i.e., both nasal and oral, interface. In turn an interface may be an indirect interface which covers the nose, mouth, or both, or an indirect interface such as an interface comprising nasal nozzles or pillows or similar which enter into the nares of the wearer.

Headgear for a respiratory interface may comprise at least two side straps which in use extend from a rear part of the headgear along the left and right sides of the patient's head to connect to the interface. Other configurations may include two sets of an upper and a lower side strap of each side.

Headgear may also comprise a top strap such as a top strap or a forehead strap, and respiratory headgear may be in various other forms. For example, headgear may comprise only a crown or forehead strap or an occipital loop, and a single strap on either side of the patient's head or face to the mask. Typically, the length of one or more of the headgear straps may be adjustable so that a patient can don the interface and headgear when the headgear strap or straps are loose and then tighten the straps when the interface and headgear are in position, to hold the mask and headgear securely in position thereafter until removal or doffing.

### SUMMARY

Patients may use various types of respiratory interfaces or masks for the provision of different respiratory therapies. To enable the respiratory therapy to be provided to the patient the interface must be retained in some way relative to either or both of the patient's mouth and nose. This is particularly the case where the respiratory therapy involves the provision of pressurised gases; the interface must be retained against the patient's face to provide at least some degree of a seal and prevent undesirable leakage of the respiratory therapy gases from the periphery of the interface. A headgear may be utilised to provide this function of retaining the interface on the patient's face.

Respiratory interfaces or masks may be used in a variety of settings, including in hospital environments and in patients' homes. Various respiratory therapies may be provided either or both when a patient is awake or while they sleep.

While a primary function of the headgear may be to retain the interface to the patient's face to counteract any pressure-generated forces and/or to create a seal with the patient's face, how the headgear transfers forces to the patient's head may significantly influence the patient's comfort and potentially also their compliance with the respiratory therapy. Load-carrying portions of the headgear may be liable to cause irritation or discomfort. This may particularly be the case where an interface and headgear are to be worn for an extended period of time, such as when sleeping.

Where an interface and headgear are used when sleeping at least some parts of the headgear may be positioned between the patient's head and the bed. This may present further opportunities for discomfort for the patient, as a localised thickness of the headgear may cause increased pressures to be experienced by their head when the headgear is laid on.

Different patients also may have significantly different anatomies. For example, they may have different circumferences of their heads, different face and skull shapes, and different tissue depths and sensitivities in different regions. This may be exemplified at the back of a patient's head, where some patients may have predominantly muscular necks or necks which taper significantly from their skulls, while other patients have more fat tissue or necks which do not taper as significantly from their skulls. These differences may result in different fits or comfort levels for a given headgear between different patients.

While a headgear may be adjusted in some ways to fit different patients, for example by shortening or lengthening one or more of the straps, such adjustments may not adequately compensate for the differences in anatomy between patients. Such adjustments may additionally or alternatively not provide for sufficient support or at least the sensation of support of the headgear on the patient's head. Ideally a headgear may be adjusted to comfortably suit the patient's anatomy, but also securely and snugly fit to the patient's head in the adjusted state.

In addition to fit constraints for different patients, it may be desirable for a headgear to perform various secondary functions in addition to retaining the interface on the patient's face. For example, it may be desirable to allow a “pull-away” function, where the headgear in a condition retaining the interface to the patient's face allows the user to pull the interface away from contact with their face. This may be to allow the patient to speak more clearly to someone, or to provide a brief respite from the therapy.

A headgear itself may have particular and localised requirements for its structure and function. For example, to retain any given respiratory interface with some force, some regions of a headgear may experience greater loads than others. There may also be location specific needs for stiffness, flexibility, softness, or any number of other properties.

Conventional headgear is commonly constructed from one or more joined sections of a laminate comprising external fabric layers which sandwich an internal foam layer, such as a natural or synthetic rubber foam. Portions of the headgear are cut from a sheet of the laminate, then connected with each other or other components to form the headgear.

An example of such a laminate material which is used in the construction of headgear is Breathe-o-prene®. Where a headgear is made from a joined sections of an existing laminate material such as Breathe-o-prene® the headgear will have a thickness of at least the three or more layers that make up the laminate. At joins between sections of the laminate the headgear will have the thickness of both joined sections. Even if efforts are made to reduce the thickness of the layers of each laminate, overlapping laminate sections can easily create undesirably thick regions and thus potential sources of discomfort for a wearer of the headgear. For example, where the laminates have three layers each, the resulting join will have a thickness of the total combined six layers.

Foam materials may deteriorate over time, leading to a loss of their properties such as their recoverability of stretching.

According to the present disclosure, a headgear may be formed by a first panel and second panel which are lapped one over the other to define a lapped region, and which are fused together at the lapped region.

One or both of the first panel and second panel may be partially not lapped by the other, and thus define one or more non-lapped regions of the headgear.

The panels may be single-ply panels. Because the headgear may be formed from individual panels comprising single plies of one material or one composite of materials, rather than from joined laminates of multiple layers of different materials. At non-lapped regions the headgear comprises only the thickness of one single-ply panel, rather than at least three plies as in the case of the use of a three-layer laminate material such as Breathe-o-prene®. This may allow for a relative reduction in thickness.

The panels may additionally or alternatively be multi-ply panels, for example a panel which is made up of multiple layers or plies of the same or different materials.

Lapped regions the headgear of the present disclosure may also have a reduced thickness, or a thickness of only two panels compared to six panels in the case of a joint of two three-layer laminate materials such as Breathe-o-prene®.

The panels may be cut to their respective desired shapes, then lapped with each other and joined together. Such a configuration may differ from conventional methods such as in the case of a laminate material such as Breathe-o-prene® where a headgear is built up by cutting already laminated panel sections, which are then joined together to form the headgear or parts of a conventional headgear.

According to the disclosure, a headgear may be formed with non-lapped portions, where the headgear comprises a single panel which is not lapped by another panel. This configuration differs from conventional methods of constructing a headgear, such as by using cut sections of Breathe-o-prene®, where the headgear will always comprise at least one three-layer fabric and foam laminate.

The panels of the headgear may be cut to shape before they are lapped together and joined.

In some forms at least some parts of the headgear may additionally or alternatively be cut once they are lapped together and potentially once one or more lapped parts are joined together.

The potential reduction in the number of panels and thickness at both lapped regions and non-lapped regions of the headgear of the present disclosure when compared to a conventional headgear made from joined laminate or multi-ply materials may provide either location-specific or overall reductions in the thickness of the headgear. Reduced thicknesses, whether in particular locations or across the whole headgear, may provide visual and/or physical perceptions to a patient of reduced bulk. Reduced thicknesses may also provide increased comfort for a patient wearing the headgear.

Any such reduction in the number of panels present at different points of the headgear and its thickness may provide corresponding decreases in the overall weight of the headgear.

The panels may exclude any foam material.

The headgear according to the disclosure may be fused, such as by welding.

Conventional headgear laminates which include foam layers are traditionally not fused by welding, as welding will degrade the cell characteristics of the foam, compressing it and causing it to become stiffer. Accordingly, welding of conventional headgear is confined to welding peripheral parts or edges of a lapped area of panels.

As the headgear according to the disclosure may exclude a foam layer or layers, and the panels may be fused together across substantial parts or even all of their overlapped areas. For example, at least a relatively wider boundary around the perimeter of a lapped region may be fused than in a conventional laminated headgear.

The exclusion, either in part or wholly, or foam materials such as Breathe-o-prene® may allow for the use of different panel materials than are required for covering a foam layer. This may for example allow the use of lower cost, thinner, and lighter panel materials.

The headgear of the disclosure may use panels made of lightweight and thin textiles. Accordingly, the overall thickness, and also the bulk and weight of a resulting headgear, may be reduced especially compared to conventional headgears constructed from laminated foams.

A headgear according to the disclosure may utilise fusing to provide the desired properties of the headgear.

By fusing across wider paths or larger areas rather than attempting to fuse only minimal areas particularly at the periphery of a lapped part of panels, it may be possible to minimise tight geometries where the panels are fused by welding, and consequently reduce the associated issues with arcing and burning that may occur when welding in tight geometries.

Two lapped panels may be fused together about at least a periphery of the lapped region.

Two lapped panels may be fused together about at least a border of the lapped region.

Two lapped panels of a rear portion for a headgear may be fused together about an entire periphery of their lapped region, except for at one or more strap connection portions of the rear portion. Where one or more strap connection portions of a rear portion are left unfused in manufacture of the rear portion, they may be fused in a in a subsequent manufacture of a headgear. For example, a strap may be interposed between the unfused first and second panels at the or each strap connection region, then the assembly fused together. By such a configuration at least the entire periphery of the lapped region of two first and second panels may be fused, where they are fused directly to each other at all but the strap connection portions, and at the strap connection portions are indirectly fused to each other by way of being fused to the interposed strap portion.

Two lapped panels of a rear portion of a headgear may be fused to each other across their entire lapped region, but for at one or more strap connection portions, at which part of a strap is interposed between the lapped first and second panels, and the first and second panels are each fused to the strap.

Two lapped panels a headgear may be fused to each other across their entire lapped region, but for at one or more pocket-forming regions of the lapped panels. An insert such as a strap end, may be provided within the pocket at the pocket-forming region before the lapped panels are fused together. Alternatively, an insert may be provided through an opening into the pocket after the lapped panels are fused together. Once an insert is provided within the pocket, the lapped panels and insert may be fused together, or remain unfused.

Two lapped panels may be fused together from a first edge of the lapped region to an opposite edge of the lapped region.

Two lapped panels may be fused together across the entire lapped region, but for minor regions within the interior of the lapped region.

Two lapped panels may be fused together at a substantial entirety of the lapped region.

Two lapped panels may be fused together across the entire lapped region.

Two lapped panels may be fused together by full-surface fusing of the panels at the lapped region.

Two lapped panels may be fused together such that a majority of the length along a line between two edges of the lapped region is fused.

Two lapped panels may be fused together such that a majority of the length along any line between two edges of the lapped region is fused.

The majority may be anywhere from a bare majority to a totality.

Reference to an edge of a headgear or part of a headgear, such as of a lapped region, will be understood to include to any point along the perimeter of that part.

In contrast to conventional stitched joins where the desire is to minimise the size of seams to reduce their impact on comfort or their visibility, panels which are joined by fusing may be fused together across large areas without the corresponding seam bulk or change to the surface finish that a large, stitched area would cause.

Where panels are fused together, their entire adjacent surfaces at the fused part may be connected to each other. Where panels are conventionally stitched together, the panels are only connected together between each successive stitch. Therefore, while stiches may even be provided closely together and across a large area, a comparative fused area of panels can provide a larger degree of connection between the panels than a stitched join. In other words, by fusing panels rather than stitching them, for a given area which is to be fused or stitched, a relatively greater proportion of the area of the adjacent panel surfaces may be secured against each other.

Panels, and particularly textile panels, which are fused across large portions of the panels may provide firm yet flexible properties to the headgear. The fused areas may provide a visually clean and uniform surface to the panels. Selectively fusing and not fusing parts of the panels may allow for different material properties within regions of the same panel or panels. It may also provide differences in the surface features of regions made up of the same panel or panels.

Fabric may be an example of a textile material.

Where two panels are lapped to form a headgear or part of a headgear, both panels may be a textile, such as a fabric.

One or both of two lapped panels may be made of or include a polymer material.

One or both of the two lapped panels may be made of or include a dipolar material.

Two panels which are fused together may be formed into one integral composite panel at the lapped region, rather than two separate panels which are otherwise bonded together or affixed to each other.

Fusing panels may present advantages over other additive methods of joining panels together, such as by stitching or the use of an adhesive. In particular, the weight and potentially also thickness of the joined panels may be relatively reduced.

Entirely or at least predominantly forming a headgear by fusing its constituent panels, such as by welding, may provide a more cost-effective method of joining panels together than other conventional methods.

Fusing may additionally or alternatively be applied to panels at a non-lapped region of one or more lapped panels to alter the properties of the panel.

A headgear according to the disclosure which has two lapped panels that define a lapped region may also have one or more non-lapped regions of one or both of the panels.

A non-lapped of one panel may form a border around part or all of the other panel. For example, a second panel may be provided wholly within the bounds of a first panel, so that a non-lapped border of the first panel extends about the second panel.

A headgear according to the disclosure may have a non-lapped region about a periphery of part or all of the headgear.

For example, a headgear may have a non-lapped region or regions about both an upper and lower periphery of a rear portion of the headgear.

The non-lapped region or regions may be formed by the same panel.

The non-lapped region at the upper periphery of the rear portion of the headgear may be provided along a substantially straight perimeter of the lapped region.

The non-lapped region at the upper periphery of the rear portion may be of a substantially continuous width.

The non-lapped region at the lower periphery of the rear portion of the headgear may be provided along one or more curved portions of the lapped region.

The non-lapped region at the lower periphery of the rear portion may be of a varying width.

The non-lapped region at the lower periphery of the rear portion may have an edge with a radius of curvature less than that of an adjacent curved portion of the lapped region.

The non-lapped region at the lower periphery of the rear portion may define one or more crescent shapes.

A headgear according to the disclosure which has a first and second lapped panels which define a lapped region may be fused about an entire perimeter of the lapped region.

The fusing about the entire perimeter of the lapped region may provide the first and second panels as a single unitary planar assembly with no free panel edges at the lapped region.

A headgear according to the disclosure may have a rear portion that includes a first and second lapped panels and a non-lapped region of the first panel which defines at least part of a perimeter of the rear portion.

The non-lapped region of the first panel comprises a border.

The border is located, when the headgear is worn, at least at upper and lower edges of the rear portion.

The border may vary in width away from the lapped region about the perimeter of the rear portion.

The border provides an edge-softening effect.

A headgear or rear portion for a headgear according to the disclosure which has a first and second lapped panels that are fused together at a lapped region of the panels may include two strap connection portions. In assembling the headgear two straps may be connected at the respective strap connection portions of the rear portion. In this configuration the lapped region may define a contiguous fused zone of the first and second panels between the two strap connection portions. The contiguous fused zone of the first and second panels aids in load transfer across the headgear between the straps of the headgear.

The lapped region may define more than one contiguous fused zone of the first and second panels between the two strap connection portions.

Where there are more than two strap connection portions, with a corresponding number of straps connected thereto, the lapped region may define corresponding contiguous fused zones between each one of the straps and another one of the straps.

Where there are more than two strap connection portions, the lapped region may define a single contiguous fused zones between each of the straps.

A headgear according to the disclosure may have a first and second drapeable textile panels which are lapped against each other to define a lapped region within which the panels are fused together at a welded portion of the second panel.

The welded portion may be the whole of the second panel at the lapped region, such that the first and second panels are fused together across the whole of the lapped region.

The whole of the second panel may be lapped by the first panel.

The second panel is a weldable textile.

The first panel is a non-weldable textile.

The second panel and the first panel may each be a weldable textile.

The first panel and second panel may each have different stretch properties.

The headgear may further comprise a strap portion configured to attach to a respiratory interface.

The first and second panels may define a non-lapped region, and at the lapped region the first and second panels respectively overlap and underlap each other and at the non-lapped region the first panel is not lapped.

Where the first panel and second panel are fused together may define a fused zone, and the headgear may have reduced stretch at the fused zone compared to at the non-lapped region of the first panel.

At the fused zone the headgear may have reduced stretch compared to at a non-fused portion of the second panel.

A headgear according to the disclosure may include a first and second drapeable panels which are lapped against each other to define a lapped region and are fused together at the lapped region.

Where the panels are fused together, their drape may be decreased.

Where the panels are fused together, the headgear may have a relatively reduced drape compared to one or more of a) the drape of the first panel, b) the drape of the second panel, or c) the drape of a non-fused part of the lapped region.

The first and second panels may be extensible, and the fused portion of the lapped region may have a relatively reduced extensibility.

The first and second panels may be extensible, and the fused portion of the lapped region is relatively inextensible.

The first and second panels may be extensible, and the fused portion of the lapped region is inextensible.

According to one aspect, the present disclosure provides a headgear comprising a plurality of panels which are lapped with each other, where the panels are joined together to define the headgear by fusion of the panels to each other.

The panels are fused by welding.

The panels are fused by radio frequency welding.

The adjacent lapped surfaces of the panels are fused to each other, not just peripheral edges of the lapped parts.

According to another aspect, the present disclosure provides a headgear comprising a first panel and second panel which are lapped with each other, where the headgear is fused across a substantial part of the lapped part of the first panel and the second panel.

The headgear is fused across a majority of the lapped part of the first panel and the second panel.

The headgear is fused about an entire periphery of the lapped parts of the first panel and second panel.

The headgear is also fused at a non-lapped part of either or both of the first panel and the second panel.

The first and second panels are exclusively joined by fusing.

The headgear is fused by radio frequency welding.

According to another aspect, the present disclosure provides a method of manufacture of a headgear for a patient interface, the method comprising the steps of:
providing a first panel and a second panel which partially overlaps the first panel to define a lapped region, and
applying a weld to the first panel and second panel,
wherein the step of applying the weld comprises applying a weld to the lapped region and past an outer perimeter of the lapped region.

The step of applying a weld further comprises applying a weld to a non-lapped region of the first panel and/or second panel, at which the panels are not lapped with each other.

According to another aspect, the present disclosure provides a headgear comprising a plurality of panels, where at least part of each panel is lapped against at least part of another panel and the lapped panels are fused together.

Combinations of panels having different properties, lapped interfaces between panels, and amounts, degrees, or configurations fused lapped parts provide for a headgear with the potential for location-specific features and properties.

According to another aspect, the present disclosure provides a headgear comprising a first panel and a second panel which are lapped with each other to define a lapped region, and the first panel and second panel are fused together about a perimeter of the lapped region.

The panels may be fused together about an entire perimeter of the lapped region.

Where the second panel lies within an extent of the first panel, the panels may be fused together about an entire perimeter of the second panel.

According to another aspect, the present disclosure provides a headgear comprising two textile panels which are lapped against each other.

The headgear consists of two textile panels and a plurality of strap fixtures.

The two textile panels are partially fused together.

The fusing is by welding.

According to another aspect, the present disclosure provides a headgear comprising two fabric panels which are lapped against each other.

The headgear consists of two fabric panels and a plurality of strap fixtures.

The two fabric panels are partially fused together.

The fusing is by welding.

According to another aspect, the present disclosure provides a headgear comprising a first panel and second panel which are lapped with each other to define a lapped region and the first panel and second panel are fused together about a perimeter of the lapped region.

According to another aspect, the present disclosure provides a headgear comprising a first panel and second panel which are lapped with each other to define a lapped region and portions the lapped region away from its perimeter are bonded together, where the bond is of a material or materials of the first panel and second panel to each other.

The panels are bonded together without any additive material such as a stitching or an adhesive.

According to another aspect, the present disclosure provides a headgear for a patient interface, the headgear comprising a first and second lapped panels, the lapped panels defining a lapped region in which the first and second panels respectively overlap and underlap each other and a non-lapped region in which the first panel is not lapped,
wherein, at the lapped region, adjacent surfaces of the respective lapped panels are fused together.

The adjacent surfaces of the respective panels the lapped region are fused directly to each other without any interposed material.

The lapped region comprises parts which are fused and parts which are non-fused.

The lapped region comprises a transition zone between a part which is fused and a part which is non-fused, the transition zone defining a degree of fusion between that of the non-fused part and the fused part.

The transition zone comprises a gradient of degrees of fusion between that of the fused part and the non-fused part.

A majority of the lapped region is fused.

A substantial entirety of the lapped region is fused.

The headgear comprises a third panel and the first panel, second panel, and third panel define the lapped region in which the first panel is respectively overlapped and underlapped by the second panel and third panel, and wherein adjacent surfaces of the first panel and second panel, and first panel and third panel, are each fused together.

The second panel is fully lapped by the first panel.

The first panel and second panel are both stretchable, and the first panel is more stretchable than the second panel.

The second panel has one or more cut-outs to define a stretch zone of the first panel within the one or more cut-outs.

The second panel is provided in two or more pieces to define one or more stretch zones of the first panel between the two or more pieces of the second panel.

Where the lapped panels are fused together defines a fused zone, and the headgear at the fused zone has one or more of a reduced stretchability, decreased thickness, or smoothed surface relative to the headgear at a non-fused zone.

Where the lapped panels are fused together defines a fused zone, and the headgear at the fused zone has reduced stretchability relative to each of the first panel and second panel.

Where the lapped panels are fused together defines a fused zone, and the headgear at the fused zone as a reduced stretchability relative to the headgear at the non-lapped region.

The lapped region comprises a plurality of discrete areas of the lapped panels.

The non-lapped region comprises a plurality of discrete areas of the first panel.

The headgear has different material properties or characteristics selected from one or more of drape, stretch properties, density, surface hardness, surface texture, and thickness at each of:
a) a fused part of the lapped panels,
b) a non-fused part of the lapped panels, and
c) the non-lapped region of the first panel.

The headgear at a fused part comprises one or more of a greater stiffness, a reduced extensibility, a lesser thickness, and a smoother surface texture than the headgear at a non-fused part.

The non-lapped region of the first panel is a first non-lapped region, and the headgear further comprises a second non-lapped region at which the second panel is not lapped.

The headgear comprises different stretch properties at each of:
a fused part of the lapped region,
a non-fused part of the lapped region, and
the non-lapped region of the first panel.

Adjacent surfaces of the first and second panels about a periphery of the lapped region are fused together.

The panels are fused together by a heating of a material of one or both of the panels.

The adjacent surfaces of the first and second panels are fused together about a whole periphery of the lapped region.

The second panel is fully lapped by the first panel, such that an area of the second panel defines the lapped region.

The second panel is fully lapped by the first panel, and the adjacent surfaces of the first and second panels are fused together about a whole periphery of the second panel.

The periphery of the second panel is a border within a perimeter of the lapped region.

Fusing is applied to the panels across a border both sides of the perimeter of the second panel.

The periphery of the lapped region is a border within a perimeter of the lapped region.

The periphery of the lapped region includes a border both sides of the perimeter of the lapped region.

The border is greater than about 2 mm in width.

The border is from about 2 mm to about 10 mm in width.

The border extends beyond the lapped region by at least about 2 mm.

The border extends beyond the lapped region by an amount at least equal to a predetermined panel placement tolerance during manufacturing.

The heating is applied across the border, such that both the first and second panels at the lapped region of the border and the or each panel at the non-lapped region of the border are affected by the heating.

The heating fuses together the first and second panels at the lapped region of the border.

Where the panels are heated a material property of the panels is altered relative to those not exposed to the heating.

The heating comprises a melting of a material of one or both of the panels.

The first and second panels at the lapped region are continuously fused together across the lapped region between two or more points about a or the perimeter of the lapped region.

According to another aspect, the present disclosure provides a headgear for a patient interface, the headgear comprising a first and second lapped panels, the lapped panels defining a lapped region in which the first and second panels respectively overlap and underlap each other, and a non-lapped region in which the first panel is not lapped,
wherein the lapped region and non-lapped region together comprise at least one first stretch zone and at least one relatively reduced second stretch zone, and
wherein each at least one second stretch zone is defined by a fused portion of the lapped region and each at least one first stretch zone is defined by a non-fused portion of either or both of the lapped region and the non-lapped region.

A fused portion of the lapped region is less stretchable than a non-fused portion of the lapped region.

A fused portion of the non-lapped region is less stretchable than a non-fused portion of the non-lapped region.

At a first stretch zone the headgear is more stretchable than the headgear at a second stretch zone.

At a second stretch zone the headgear is substantially non-stretch.

The first layer is a stretch layer, and the second layer is a relatively lower stretch layer.

Each of the lapped region and non-lapped region comprise each of a stretch zone and a non-stretch zone.

According to another aspect, the present disclosure provides a headgear for a patient interface, the headgear having a central section comprising a first layer and a second layer and defining at least one stretch region where the first layer is not lapped by the second layer and at least one reduced stretch region where the first layer is lapped by the second layer,
wherein the stretch region is located between laterally spaced strap connection portions of the central section.

The central section comprises a reduced stretch region at an upper portion thereof, and the reduced stretch region connects to lateral sections of the headgear either side of the central section.

One or more reduced stretch regions of the central section and lateral sections define a band of the headgear.

The reduced stretch region extends beyond the bounds of where the first layer is lapped by the second layer.

The layer or layers of the headgear at the reduced stretch region or regions are welded.

The headgear further comprises one or more lapped stretch regions where the first layer is lapped by the second layer, and wherein at each one or more lapped stretch region the headgear is unwelded.

A lateral extent of each lateral region defines one or more strap connection portions.

Each of the one or more strap connection portions have an increased width relative to a part of the band at each respective lateral region.

The one or more strap connection portions each include at least one of the one or more lapped reduced stretch regions.

The headgear has a lateral dimension along the band and a width dimension perpendicular thereto, and at one or both of the lateral sections the second layer is about 60% to about 95% of the width of the first layer.

At one or both of the lateral sections the second layer is about 70% to about 80% of the width of the first layer.

At one or both of the lateral sections the second layer is about 80% to about 90% of the width of the first layer.

The headgear has a lateral dimension along the band and a width dimension perpendicular thereto, and at a lateral middle of the stretch region the second layer is about 20% to about 70% of the width of the first layer.

At the lateral middle of the stretch region the second layer is about 40% to about 60% of the width of the first layer.

One or more of the one or more strap connection portions laterally tapers away from the strap.

One or more of the one or more strap connection portions have a triangular shape.

The one or more strap connection portions include a strap, and the strap connection portion and strap are comprised by a stretch panel and/or a relatively reduced stretch panel.

The stretch region of the central section is located between the band and two strap connection portions.

The lapped region is continuous.

The non-lapped region is continuous.

The first layer is of a first stretch value and the second material is of a second lesser stretch value.

The first layer is a stretch layer and the second layer is a relatively reduced stretch layer.

The first layer is a stretch layer and the second layer is a non-stretch layer.

According to another aspect, the present disclosure provides a headgear for a patient interface, wherein the headgear has a band portion and a plurality of strap connection portions and comprises a plurality of lapped panels which define at least one lapped region in which at least two of the plurality of panels respectively overlap and underlap each other and at least one non-lapped region in which one or more of the plurality of panels is not lapped by another of the plurality of panels,
wherein the headgear is welded at each at least one lapped region to fused ether adjacent surfaces of the lapped panels, and
a) the band portion has a first arrangement of welded and non-welded adjacent panel surfaces of one or more lapped regions within the band, and
b) one or more of the plurality of strap connection portions has a second arrangement, different from the first arrangement of welded and non-welded adjacent panel surfaces of one or more lapped regions within the or each respective one or more of the plurality of strap connections.

The first arrangement comprises substantially entirely welded adjacent panel surfaces.

For example, adjacent panels surfaces may be welded to each other except for parts minor internal regions of the headgear.

Adjacent panel surfaces may be welded to each other except for parts which define a desired surface texture.

The second arrangement comprises welded adjacent panel surfaces with one or more non-welded zones.

The one or more non-welded zones are located within a boundary from an edge of any lapped region within the one or more of the plurality of strap connection portions.

The boundary is at least 2 mm.

The boundary is from about 2 mm to about 10 mm.

The one or more non-welded zones further comprise one or more spot welds to limit separation of lapped panels within the or each of the one or more non-welded zones.

Welded regions of the headgear extend beyond a perimeter of each at least one lapped region.

Welded regions of the headgear include a border zone of any non-lapped region adjacent to a lapped region.

The border is from about 2 mm to about 10 mm in width.

The border extends beyond the lapped region by at least about 2 mm.

The border extends beyond the lapped region by an amount at least equal to a predetermined panel placement tolerance during manufacturing.

A welded part of a lapped region comprises different material properties from a non-welded part of the same lapped region.

A welded part of a non-lapped region comprises different material properties from a non-welded part of the same non-lapped region.

The headgear further comprises a plurality of straps corresponding to the plurality of strap connection portions, and each of the plurality of straps are comprised by one or more of the plurality of lapped panels.

The headgear further comprises a plurality of straps, one for connection to each of the plurality of strap connection portions.

The plurality of panels comprise a first panel and a second panel which is fully lapped by the first panel, the first panel and second panel defining a lapped region and at least one non-lapped region of the first panel, and wherein the lapped region comprises each of the band and at least two of the plurality of strap connection portions.

Each of the plurality of strap connection portions depend from the band.

At least one pair of the strap connection portions expand laterally from where a strap is to be connected towards the band.

The band further comprises one or a pair of top straps.

According to another aspect, the present disclosure provides a headgear for a patient interface, the headgear comprising a first stretch material and a second stretch material in respective panels which are at least partially lapped with each other,
wherein the lapped panels of the first stretch material and second stretch material are joined together at a joined region such that they have low stretch.

The first stretch material is more stretchable than the second stretch material.

At the joined region the headgear is less stretchable than that of either the first stretch material alone or the second stretch material alone.

At the joined region the headgear is less stretchable than the lapped first stretch material and second stretch material at a non-joined region.

The low stretch where the lapped panels are joined together is a relatively lower stretch than at a lapped panels are not joined together.

At the joined region the headgear is of a reduced stretch relative to a non-joined region.

At the joined region the headgear is substantially non-stretch.

The lapped panels of the first stretch material and second stretch material are joined together at the joined region by fusion of the panels together.

The lapped panels of the first stretch material and second stretch material are joined together at the joined region by fusion by melting of one of the materials into the other of the materials.

According to another aspect, the present disclosure provides a method of manufacture of a headgear for a patient interface, the method comprising the steps of:
providing a first panel and a second panel, the panels defining a lapped region in which the first and second panels respectively overlap and underlap each other and a non-lapped region in which the first panel is not lapped, and
melting one or both of the panels at the lapped region so as to fuse the panels together and define a fused zone of the lapped region.

The method further comprises the step of melting the first panel at the non-lapped region to define a fused zone of the non-lapped region.

The first panel is melted to define a fused zone about a perimeter of the lapped region.

The step of melting one or both of the panel sat the lapped region and the step of melting the first panel at the non-lapped region are provided as a single operation.

The step or steps of melting comprise the application of a heat and pressure to the first and/or second panels.

The first panel has a first melting point, and the second panel has a second melting point, and the step or steps of melting comprise raising the temperature of at least part of the first panel and second panel above both of the first melting point and the second melting point.

The first melting point is higher than the second melting point.

The step or steps of melting comprise raising the temperature of at least part of the first panel and second panel to temperature above the second melting point but below the first melting point.

The step or steps of melting comprise the application of a heat and pressure to both of the first panel and the second panel, but only the second panel is melted.

The step or steps of melting comprise a welding.

The first panel and second panel are provided between a first and second dies.

The method further comprises the step of moving the first and second dies towards each other.

The step of moving the first and second dies towards each other is comprised by the step or steps of melting.

The first die comprises a first relatively raised level and second relatively recessed level, wherein the first level defines the fused zone or zones, and the second level defines an unfused zone at which the panel or panels of the headgear are not melted.

The first die comprises a substantially immediate transition between the first level and the second level.

The first die comprises a graded transition between the first level and the second level.

The graded transition of the first die defines a transition zone between the fused zone and the unfused zone of the headgear.

The level of the graded transition of the first die varies linearly between the first level and the second level.

The level of the graded transition of the first mould palate follows an s-curve shape.

The first level and second level define in relief an indicia, such that when melted the headgear comprises one or more fused zones and unfused zones which represent the indicia.

The method further comprises the step of cutting one or both of the first panel and the second panel to a predetermined size and/or shape.

One or both of the dies comprise a cutting element, and the step of cutting is combined with the step of moving the first and second dies towards each other.

The step of melting one or both of the panels at the lapped region occurs after a bringing together of the two dies with the first panel and second panel therebetween.

The step of melting the first panel at the non-lapped region occurs after a bringing together of the two dies with the first panel and second panel therebetween.

The first panel is more stretchable than the second panel.

One of the first panel and the second panel has a higher melting point than the other of the first panel and second panel.

According to another aspect the disclosure provides a rear portion of a headgear, the rear portion comprising a first and second lapped panels, the lapped panels defining a lapped region in which the first and second panels respectively overlap and underlap each other and a non-lapped region in which the first panel is not lapped, wherein, at the lapped region, adjacent surfaces of the respective lapped panels are fused together.

Lateral ends of the lapped region are remain unfused for receiving between the first panel and second panel a headgear strap.

According to another aspect the disclosure provides for a headgear comprising the rear portion and a pair of straps, each strap sandwiched between the first panel and second panel at the lateral ends of the lapped region.

The lapped first panel, second panel, and strap are fused together at each lateral end of the lapped region.

According to another aspect, the present disclosure provides a headgear comprising a plurality of panels, where at least part of each panel is lapped against at least part of another panel they are joined together to provide a thin and seamless or substantially seamless headgear.

The lapped parts of each panel are joined together by a fusing of one or both panels into the other or each other of the panels.

According to another aspect, the present disclosure provides a headgear comprising a plurality of panels, where at least part of each panel is lapped against at least part of another panel they are fused together to provide a lightweight headgear.

According to another aspect, the present disclosure provides a headgear comprising a plurality of panels, where at least part of each panel is lapped against at least part of another panel they are fused together, wherein the panels are configured such that the headgear retains at least some of its in-use shape when in an at-rest state.

At least one of the plurality of panels comprises a different material to that of another panel of the plurality of panels.

At least one of the panels at a lapped region comprises a different material to that of another panel at a lapped region.

The plurality of panels comprise a first panel and a second panel, and the first panel comprises a different material to that of the second panel.

The plurality of panels further comprise a third panel, and the second and third panels comprise the same material, which is a different material to that of the first panel.

Different materials comprise one or more of a different texture, softness, stretch properties including one or more of in-plane stiffness, out-of-plane flexibility, and recoverability, density, thickness, colour, frictional coefficient in relation to a reference material, breathability, or degree of transparency or sheer.

Different materials comprise one or more of a directionally different: texture, softness, stretch property including one or more of in-plane stiffness, out-of-plane flexibility, and recoverability, or frictional coefficient in relation to a reference material.

The two opposed major faces of at least one panel of the plurality of panels comprises one or more of a different texture, frictional coefficient, or colour of the opposed major faces of the panel.

Exposed portions of major faces of the plurality of panels define an internal surface and an external surface of the headgear relative to the head of a patient in use, and wherein at least some of the plurality of panels each define part of the internal surface and/or the external surface of the headgear.

The at least some of the plurality of panels comprise one or more of a different texture, softness, colour, or frictional coefficient in relation to a reference material.

The at least some of the panels defining part of the internal surface of the headgear comprise a greater surface softness than that of at least some of the panels defining part of the external surface of the headgear.

Only some of the plurality of panels define the internal surface and only some of the plurality of panels define the external surface.

Excluding the or each at least one non-lapped region, an exposed portion of a panel either defines part of one or the other of the internal surface and external surface.

The plurality of panels comprise a panel of first stretch material and a panel of second stretch material, where the stretchability of the first stretch material and the second stretch material are different.

The first stretch material is more stretchable than the second stretch material.

The plurality of panels comprise at least one elasticated textile panel and at least one non-elasticated textile panel.

The plurality of panels comprise:
a rear portion for location at the rear of the head of a patient,
a top strap, and
at least two side straps for attachment to the patient interface.

The rear portion transfers loads between the at least two side straps, and the rear portion comprises at least one panel of a first material which laps a portion of a side strap panel of each of the at least two side straps.

The rear portion comprises a plurality of panels of the first material, and each of the at least two side straps are lapped on respective sides by panels of the first material.

An extent of the rear portion to be located lowest on the rear of the head of a user comprises a lower panel of a second material.

The second material is non-unravelable material.

The lower panel is lapped to or between one or more of the plurality of panels of the first material.

The lower panel of the second material is more stretchable than the plurality of panels of the first material.

The lower panel of the first material is a stretch panel and the plurality of panels of the first material are non-stretch panels.

The lower panel of the second material is elasticised, and the plurality of panels of the first material are non-elasticised.

The lower panel of the second material has a greater elasticity than the plurality of panels of the first material.

The second material is an elastic material, and first material is a substantially inelastic material.

A portion of the lower panel is in a non-lapped region of the headgear, and the portion of the lower panel is substantially crescent-shaped.

At least a portion of the lower panel within a non-lapped region of the headgear is substantially crescent-shaped, having a concave edge and a convex edge, and wherein the concave edge forms at least a portion of a lower peripheral edge of the rear portion.

The rear portion comprises an upper edge panel, the upper edge panel forming at least a portion of an upper peripheral edge of the rear portion, the upper edge panel comprising a material that is thinner and/or softer than that of a panel to which it is lapped.

The upper edge panel is lapped to another panel at the rear portion so as to define an internal surface, relative to the head of a patient in use, of the headgear at an upper region of the rear portion.

The upper edge panel is provided within both a lapped region and a non-lapped region, and wherein the non-lapped region forms at least part of an upper peripheral edge of the rear portion.

The upper edge panel at the non-lapped region is configured, in use and towards the upper peripheral edge of the rear portion, to roll away from the head of the patient.

The upper edge panel at the non-lapped region extends away from an adjacent lapped region a distance of about 5 times to about 20 times the thickness of the upper edge panel.

The top strap defines an internal surface oriented towards the head of the patient in use and an external surface oriented away from the head of the patient in use, and wherein the internal surface has a greater softness than that of the external surface.

The top strap depends from the rear portion.

The top strap depends from the rear portion and at least one side strap of each lateral side of the rear portion and/or top strap.

The top strap has a width greater than that of the at least two side straps.

The top strap is of a different colour to that of the at least two side straps.

The top strap comprises a pair of top strap portions which are adjustably fixable to each other to provide a variable-length top strap, each top strap portion comprising an internal top strap portion panel and an external top strap portion panel adhesively bonded to each other.

The internal top strap portion panels have a greater softness than that of the external top strap portion panels.

The internal and external top strap portion panels respectively overlap and underlap the panel or panels of the rear portion and/or side straps to which they are lapped.

The top strap is relatively stiffer and/or denser than the rear portion.

The top strap comprises one or more panels of a material having a greater stiffness and/or greater density than that of one or more panels comprising the rear portion.

The at least two side straps comprise two lateral sets of an upper and a lower side strap, each set of side straps for connection to corresponding sides of the patient interface.

The two upper side straps comprise a unitary panel which extends across the rear portion of the headgear.

The unitary panel is provided within a lapped region across at least part of the rear portion of the headgear and within a pair of distal non-lapped regions.

An upper and lower side strap comprise a unitary panel, the unitary panel configured to extend around a rear of the ear of a patient.

A lapped region of the upper side straps is respectively overlapped and underlapped by panels of the top strap.

The lower side straps are overlapped and underlapped by a plurality of panels of the rear portion.

An ear loop for passing behind the ear of a patient defines a lateral peripheral edge of the rear portion between each lateral set of upper and lower side straps.

The edge profile of each ear loop comprises a pair of straight-line portions.

The pair of straight-line portions form a V-shape, the tip of each V directed towards the other and into the rear portion of the headgear.

The two upper side straps comprise two respective panels, each provided within a lapping region at the rear portion of the headgear and within distal non-lapping regions.

Each of the two respective panels of the two upper side straps are overlapped and underlapped by panels of the rear portion of the headgear.

Terminal portions of either or both of the upper and lower side straps include gripping tabs comprising a first and second tab panels, the tab panels respectively overlapping and underlapping the terminal portion at a first tab region and lapping each other at a more distal second tab region.

The first and second tab panels are of a material that is one or more of thinner, softer, of a different colour, or has a lower coefficient of friction with respect to a reference material than that of the material of the respective straps of the sets of upper and lower side straps.

The first and second tab panels are of a plastics material that is one or more of thinner, harder, stiffer, of a different colour, or has a lower coefficient of friction with respect to a reference material than that of the material of the respective straps of the sets of upper and lower side straps.

The headgear at the second more distal tab region is of greater stiffness than the headgear at the first tab region.

The second more distal tab region is thinner than one or more of the upper and lower side straps at a non-lapped region.

The gripping tabs further comprise at one external surface a first half of a hook and loop fastener.

The side straps are configured such that the strap surface which corresponds to the first half of the hook and loop fastener, when the strap is folded back on itself, comprises a second half of the hook and loop fastener.

The at least two side straps comprise a left and right side straps, each side strap for connection to a corresponding side of the patient interface.

Each side strap of the set of side straps is configured to fold back onto and attach to itself to define a connection loop by which the patient interface may be retained.

Each side strap of the set of side straps comprises a series of visual features along at least one surface of the side strap, the visual features for indicating adjustment points to the patient when folding a side strap back onto itself.

The visual features are regularly spaced along the surface of each side strap.

Each side strap of the set of side straps comprises a series of tactile features along at least one surface of the side strap, the tactile features for providing a tactile feedback to a patient of different adjustment conditions of the respective strap.

The series of tactile features provide for indexing of the adjustment of each respective side strap.

An interaction of the tactile features with the patent interface provides the tactile feedback to the patient.

Each side strap comprises a strap panel, the strap panel lapped on at least one major face with a tactile feedback panel, wherein the tactile feedback panel is one or more of thinner, harder, and stiffer than the strap panel which it laps and wherein the tactile feedback panel and the strap panel are fused together.

The tactile feedback panel comprises the tactile features, the tactile features provided by a series of voids through the tactile feedback panel.

The series of voids present the tactile feedback panel as having a series of ridges relative to the strap panel to which it is lapped.

The tactile feedback panel is harder and/or stiffer than the strap panel, and the series of ridges are for mechanically engaging with a buckle of the patent interface.

Each respective lateral extent of the top strap and rear portion non-lappingly interface with each other and an end of a respective one of the side straps.

Each respective lateral extent of the top strap and rear portion and end of the respective side strap interface with each other in an edge-to-edge configuration.

Each respective lateral extent of the top strap and rear portion and end of the respective side strap are lapped on at least one set of major faces by a joint panel.

At least one major face of one or more of the top strap, rear portion, and side straps is lapped along at least a portion towards their peripheries with a stiffening panel.

The stiffening panel comprises a stretch material which is less stretchable than the respective one or more of the top strap, rear portion, and side straps.

The stiffening panel comprises a relatively reduced stretch material.

The stiffening panel comprises a low stretch material.

The stiffening panel comprises a non-stretch material.

The stiffening panel comprises a substantially inextensible material.

The stiffening panel comprises a non-elasticated material.

The top strap comprises a single unitary strap.

The rear portion comprises a single unitary strap.

The top strap and rear portion comprise a closed loop.

The set of side straps depend from intersections of the top strap and rear portion.

The intersections of the top strap and the rear portion are, in use, located above the ear of a patient.

One or more of the set of side straps, the rear portion, and the top strap comprise at least in part a first stretch panel which is overlapped and underlapped by a pair of second stretch panels, where the first stretch panel is more stretchable than the second stretch panels.

One or more of the set of side straps, the rear portion, and the top strap comprise at least in part a relatively elastic panel which is overlapped and underlapped by respective relatively inelastic panels.

An entirety of one or both of the top strap and rear portion comprise a first stretch panel which is overlapped and underlapped by a pair of second stretch panels, where the first stretch panel is more stretchable than the second stretch panels.

An entirety of one or both of the top strap and rear portion comprise a relatively elastic panel which is overlapped and underlapped by respective relatively inelastic panels.

The rear portion and top strap form a closed loop, and the rear portion and top strap together comprise a relatively greater stretch panel and a relatively lesser stretch panel.

The rear portion and top strap comprise two relatively lesser stretch panels, the two relatively lesser stretch panels each at a respective first end are lapped with a relatively greater stretch panel and at the other ends are lapped with each other.

The rear portion and crown portion comprise a single relatively lesser stretch panel, the single relatively lesser stretch panel lapped at two lateral ends to one or more relatively greater stretch panels.

Terminal portions of either or both of the upper and lower side straps include gripping tabs comprising a first and second tab panels, the tab panels respectively overlapping and underlapping the terminal portion at a first tab region and lapping each other at a more distal second tab region.

The first and second tab panels are of a material that is one or more of thinner, softer, of a different colour, or has a lower coefficient of friction than that of the material of the respective straps of the sets of upper and lower side straps.

The headgear at the second more distal tab region is of greater stiffness than the headgear at the first tab region.

The gripping tabs further comprise at one external surface a first half of a hook and loop fastener.

The side straps are configured such that the strap surface which corresponds to the first half of the hook and loop fastener, when the strap is folded back on itself, comprises a second half of the hook and loop fastener.

One or more of the plurality of panels comprise an unravelable material.

At least part of an edge of the headgear is treated by a conditioner.

An edge portion which is treated by the conditioner comprises increased fray resistance relative to a non-adhesive lapped edge portion of the same material.

At least part of an edge of the headgear is rolled back onto itself.

An adhesive is provided between the rolled-back portion and the portion onto which it is rolled back.

At least part of an edge of the headgear comprises an edge softening panel, a portion of which is lapped to a more interior panel, and wherein the edge panel comprises a material that is thinner and/or softer than that of the more interior panel to which it is lapped.

The edge panel is lapped to the more interior panel such that the edge panel defines an internal surface, relative to the head of a patient in use, of the headgear.

The edge panel is configured, in use and towards its outer end, to roll away from the head of the patient.

The edge panel at the non-lapped region extends away from an adjacent lapped region a distance of about 5 times to about 20 times the thickness of the more interior panel to which it is lapped.

The headgear has a weight of less than about 30 g.

The headgear has a weight of less than about 20 g.

The headgear has a weight of less than about 10 g.

The headgear has a weight of about 15 g to about 30 g.

The headgear has a weight of about 17.5 to about 27.5 g.

The headgear has a weight of about 25 g.

One or more of the plurality of panels comprise planar panels.

One or more of the plurality of panels comprise tubular panels.

According to another aspect, the disclosure provides a headgear comprising a first panel which includes a selectively fused zone, wherein in the selectively fused zone the first panel is contiguously fused along a first direction and at least in parts discontiguously fused along a second direction, the second direction being a different direction to the first direction.

Within the selectively fused zone and in the first direction all fused parts of the first panel are contiguous with each other.

Within the selectively fused zone and in the second direction at least some fused parts of the first panel are non-contiguous with each other.

Within the selectively fused zone and in the second direction the fused zone is non-contiguous.

The headgear comprises a second panel at least partially lapped with the first panel, and the selectively fused zone includes at least part of the lapped panels.

At the selectively fused zone the headgear has a reduced extensibility in the first direction relative to the second direction.

The second direction is substantially perpendicular to the first direction.

The selectively fused zone is provided to one or more elongate portions of the headgear, and the first direction is taken transverse to the length of each elongate portion and the second direction is taken along the length of each elongate portion.

The elongate portions comprise a plurality of straps.

The elongate portions comprise a rear loop.

The selectively fused zone is provided at a bifurcated portion of the headgear and the second direction is taken across the bifurcation and the first direction is taken along respective sides of the bifurcation.

The first direction is a direction of load transfer between two parts of the headgear, and the second direction is substantially perpendicular to the direction of load transfer.

The headgear comprises a first selectively fused zone between lateral sides of a rear portion of the headgear and a second and third selectively fused zones each between respective lower strap connection portions of the rear portion and the first selectively fused zone.

The selectively fused zone is configured such that when coupled to an interface the headgear defines a contiguous loop of fused material from a first side of the interface around the patient's head and to a second side of the interface.

According to another aspect, the present disclosure provides a headgear for a patient interface, the headgear comprising a first and second lapped panels, the lapped panels defining a lapped region in which the first and second panels respectively overlap and underlap each other and a non-lapped region in which the first panel is not lapped, wherein the panels are fused together at the lapped region, and wherein a width of the non-lapped region varies.

The non-lapped region is relatively more stretchable than the lapped region.

The first panel is a stretch panel and the second panel is a non-stretch or relatively reduced stretch panel.

The headgear comprises one or more stretch zones, and at the or each stretch zone the width of the non-lapped region is locally increased.

The non-lapped region has a locally increased width at ear loops of the headgear which are to be located, in use, around the ears of the patient.

The greatest width of the non-lapped region at the ear loops of the headgear is at a region to be located, in use, above and behind the ears of the patient.

At the ear loops the ratio of total width of the non-lapped region or regions to the lapped region is from about 1:1 to about 3:1.

The non-lapped region at an upper side of a rear portion of the headgear has a substantially constant width, and the width of the non-lapped region at a lower side of the rear portion of the headgear varies.

According to another aspect, the disclosure provides a headgear for a patient interface, the headgear comprising a first and second lapped panels, the lapped panels defining a lapped region in which the first and second panels respectively overlap and underlap each other, wherein a filament is provided between the first and second panels at the lapped region and the panels are fused together either side of the filament.

The filament is able to be drawn between panels.

The filament extends around a loop of the headgear between two patient interface connecting ends of the headgear, and the filament is drawable between the panels in order adjust a fit of the headgear.

According to another aspect, the disclosure provides a headgear for a patient interface, the headgear having three stiffening structures at each side of the headgear:
a) a first stiffening structure to provide stiffness between a rear portion and a top strap of the headgear,
b) a second stiffening structure to provide stiffness between the top strap and an upper side strap of the headgear, and
c) a third stiffening structure to provide stiffness between the rear portion and a lower sides trap of the headgear,
wherein the three stiffening structures are formed by a fusing of the headgear at each of the respective locations.

The headgear comprises a first panel and the fusing of the headgear comprises a fusing of the first panel.

The headgear further comprises a second panel which is at least partially lapped with the first panel, and the fusing comprises at least in part a fusing of the lapped first panel and second panel.

The panel or panels comprise a textile including a polymer material, and the panel or panels when fused form a solid plastic.

The three stiffening structures are formed by a fusing of the panel or panels, exclusive of any additional component provided to the panel or panels.

The three stiffening structures are contiguous with each other.

According to another aspect, the present disclosure provides a headgear for a patient interface, the headgear comprising a rear portion that has a first and second lapped panels, and at least two straps, wherein each of the at least two straps are fused to only the first panel of the rear portion.

The second panel does not overlap any of the at least two straps.

According to another aspect, the present disclosure provides a headgear for a patient interface, the headgear comprising a rear portion that has a first and second lapped panels and a plurality of straps lapped and fused to the rear portion, wherein the parts of each of the straps that are not lapped to the rear portion are straight.

The parts of each of the straps that are not lapped and to the rear portion are of constant width.

Both the parts of each strap that are lapped and are not lapped to the rear portion are straight and/or of constant width.

According to another aspect, the present disclosure provides a headgear for a patient interface, the headgear comprising:
a rear portion that has a first and second lapped panels,
at least one slot cut in the second panel, and
at least one strap, each strap having a strap end inserted through a respective slot so as to be located between the lapped first panel and second panel,
wherein each at least one strap and the rear portion are joined by a fusing of the first and second panels and each at least one strap end.

According to another aspect, the present disclosure provides a headgear for a patient interface, the headgear comprising:
a rear portion that has a first and second lapped panels,
at least one slot cut in the second panel, and
at least one strap, each strap having a strap end inserted through a respective slot and folded back onto itself.

Each at least one strap is joined to the rear portion by being fused to itself at the folded back portion.

Each at least one strap is joined to the rear portion by a releasable connection of the strap to itself at the folded back portion.

The at least one slot is cut through both the first panel and second panel.

According to another aspect, the present disclosure provides a headgear for a patient interface, the headgear comprising:
a rear portion that has a first and second lapped panels, and
a top strap with a thinned central region that is sandwiched between the first and second lapped panels of the rear portion,
wherein the rear portion and top strap are joined together by fusing.

The top strap before incorporation into the headgear has a straight shape.

The top strap when sandwiched between the first and second panels has a curved shape.

According to another aspect, the present disclosure provides a headgear for a patient interface, the headgear comprising:
a rear portion that has a first and second lapped panels,
a plurality of straps, and
a strap material layer overlaid on the lapped first and second panels, wherein the strap material layer comprises the same material as each of the plurality of straps, and the plurality of strap are each lapped against the strap material layer and fused thereto.

The headgear comprises a plurality of strap material layers, each overlaid on the lapped first and second panels and having at least one strap lapped and fused thereto.
The strap material layer or layers are lapped only against the second panel of the rear portion.

According to another aspect, the present disclosure provides a headgear for a patient interface, the headgear comprising:
a rear portion that has a first and second lapped panels,
a top strap panel partially lapped against the rear portion, the top strap panel comprising a slot, and
a strap extending through the slot and being secured back onto itself.

The headgear has left and right top strap panels, each partially lapped against respective left and right sides of the rear portion, and each top strap panel comprises a slot with a respective strap extending therethrough and being secured back onto itself.

According to another aspect, the present disclosure provides a headgear for a patient interface, the headgear comprising:
a rear portion that has a first and second lapped panels,
two straps, and
two overmold components, wherein the overmold components are each overmolded over a respective one of the two straps to secure the rear portion and straps together.

The rear portion comprises two slots through the rear portion, the overmold components each further comprise a mounting post having a base and a head, wherein the head of a respective mounting post is inserted through each respective slot to connect the rear portion and overmold components together.

Each head comprises lateral wings which are wider than a respective slot.

The headgear includes four straps, and two straps are overmolded by each of the overmold components.

The rear portion comprises two slots through the rear portion, and respective portions of each of the overmold components are fused to each other through each respective slot.

According to another aspect, the present disclosure provides a headgear for a patient interface, the headgear comprising:
a rear portion that has a first and second lapped panels and defines a strap connection region, and
a strap located at the strap connection region,
wherein at the strap connection region the rear portion is folded over the strap.

The strap is joined to the rear portion by fusing the rear portion to both sides of the strap.

According to another aspect, the present disclosure provides a rear portion of or for a headgear which is for connection to a patient interface, the rear portion comprising a first and second lapped panels defining a lapped region, and wherein, except for at one or more strap connection regions, an entire perimeter of the lapped region of the first and second lapped panels are fused together.

A headgear is formed by interposing part of a strap between the first and second lapped panels at each strap connection region and fusing the panels and strap together.

The first and second panels about the entire perimeter of the lapped region of the headgear are fused together.

According to another aspect, the present disclosure provides a rear portion of or for a headgear which is for connection to a patient interface, the rear portion comprising a first and second lapped panels defining a lapped region, where, except for at one or more strap connection regions, an entire periphery of the lapped parts of the first and second panels are fused together.

A headgear is formed by interposing part of a strap between the first and second lapped panels at each strap connection region and fusing the panels and strap together.

The first and second lapped panels about the entire periphery of the lapped region are fused together.

The first and second panels at the lapped region of the headgear are entirely fused together.

According to another aspect, the present disclosure provides a headgear comprising a first and second lapped panels, the lapped panels defining a lapped region in which the first and second panels respectively overlap and underlap each other, wherein the lapped panels are fused together such that a straight-line condition between two edges of the lapped region is satisfied, wherein according to the straight-line condition a total length along a line between the chosen two edges of the lapped region which is fused exceeds a total length between the two edges which is unfused.

The straight-line condition is satisfied between at least one pair of respective upper and lower edges of a lapped region of a rear portion of the headgear.

The straight-line condition is satisfied along any straight line between an upper and lower edge of a lapped region of a rear portion of the headgear.

The straight-line condition is satisfied along any line between two edges of a lapped region of a rear portion of the headgear.

The headgear further defines a non-lapped region in which the first panel is not lapped.

Along the line between the edges of the lapped region fused portions are located at both ends of the line.

Along the line between the edges of the lapped region non-fused portions are located only away both ends of the line.

Along the line between the edges of the lapped region a cumulative length along the line at its ends where the lapped portion is fused exceeds a cumulative length along the line where the lapped portion is not fused.

Along the line between the edges of the lapped region a cumulative length along the line at its ends where the lapped portion is fused exceeds a cumulative length along the line at a central portion where the lapped portion is not fused.

While the headgear is generally referred to in the context of a patient, it will be understood that the term patient may as appropriate be substituted for an assistant to the patient or a medical professional or otherwise anyone else who may use or interact with the headgear, whether for their own use or in association with aiding someone else's use of the headgear.

As used herein the term “and/or” means “and” or “or”, or both.

As used herein “(s)” following a noun means the plural and/or singular forms of the noun.

For the purposes of this specification, the term “plastic” shall be construed to mean a general term for a wide range of synthetic or semisynthetic polymerization products, and generally consisting of a hydrocarbon-based polymer.

For the purpose of this specification, where method steps are described in sequence, the sequence does not necessarily mean that the steps are to be chronologically ordered in that sequence, unless there is no other logical manner of interpreting the sequence.

The term “comprising” as used in the specification and claims means “consisting at least in part of.” When interpreting each statement in this specification that includes the term “comprising,” features other than that or those prefaced by the term may also be present. Related terms “comprise” and “comprises” are to be interpreted in the same manner.

Other aspects of the invention may become apparent from the following description which is given by way of example only and with reference to the accompanying drawings.

### BRIEF DESCRIPTION OF THE DRAWINGS

Preferred embodiments of the invention will be described by way of example only and with reference to the drawings, in which:

FIGS. 1A and 1B are cross-sectional views of, respectively, a pre-fused and fused configuration of two panels.

FIGS. 2A and 2B are cross-sectional views of another pre-fused and fused configuration of two panels.

FIGS. 3A and 3B are cross-sectional views of another pre-fused and fused configuration of two panels.

FIGS. 4A and 4B are cross-sectional views of a pre-fused and fused configuration of three panels.

FIGS. 5A and 5B are pre- and post-fusing cross-sectional views of two panels.

FIG. 6A is a cross-sectional view of a pre-fused configuration of three panels.

FIGS. 6B and 6C show two different fused configurations of the three panels of FIG. 6A.

FIG. 7A is a plane view of two panels lapped with each other.

FIG. 7B shows a region about the perimeter of the lapped part of the two panels of FIG. 7A where the two panels are to be joined together.

FIG. 7C shows the assembly of FIG. 7B, but where the panels and the joining region are relatively misaligned.

FIG. 8A is a plane view of two panels lapped with each other.

FIG. 8B shows a region across the lapped part of the two panels of FIG. 7A where the two panels are to be joined together.

FIG. 8C shows the assembly of FIG. 7B, but where the panels and the joining region are relatively misaligned.

FIG. 9A is a side view of a welding press for welding a headgear, where the weld press is open.

FIG. 9B is a side view of a welding press for welding a headgear, where the welding press is closed.

FIG. 10 is a view of a headgear retaining an interface to the face of a patient.

FIG. 11 is a lay-flat view of a headgear.

FIG. 12 is a view of two panels.

FIG. 13 is a view of the two panels of FIG. 12 lapped with each other to define part of a headgear.

FIG. 14 is a view of the headgear of FIG. 13 where the panels have been fused together.

FIG. 15 is a view of a headgear illustrating a boundary within which a fusing may be applied to the panels of a headgear.

FIG. 16 is a close-up view of a part of the headgear of FIG. 14, showing the two lapped panels and the boundary of where fusing is applied to.

FIG. 17A is a lay-flat view of a headgear.

FIG. 17B is a lay-flat view of the headgear of FIG. 17A with straps joined to it.

FIG. 18 is a lay-flat view of a headgear.

FIG. 18-1 is a lay-flat view of a headgear.

FIG. 18-2 is a view of the headgear of FIG. 18-1 worn by a patient.

FIG. 18-3 is a view of four different rear portions for a headgear.

FIG. 19 is a lay-flat view of a headgear.

FIG. 20 is a view of a die to form the headgear of FIG. 19.

FIG. 21 is a lay-flat view of a headgear.

FIG. 22 is a view of a die to form the headgear of FIG. 21.

FIG. 23 is a lay-flat view of a headgear.

FIG. 24 is a view of a die to form the headgear of FIG. 23.

FIG. 25 is a view of another configuration of a die having indicia-forming elements.

FIG. 26 is a view of part of a headgear having indicia formed in it.

FIG. 26-1A is a view of a headgear and a patient interface.

FIG. 26-1B is a cross-sectional view through the line A-A of FIG. 26-1A.

FIG. 27 is a lay-flat view of a headgear having indicia formed in it.

FIG. 28A is a pre-fusing lay-flat view of a headgear.

FIG. 28B is a view of the headgear of FIG. 28A after it has been fused.

FIG. 29 is a lay-flat view of a headgear.

FIG. 30 is a lay-flat view of a headgear.

FIG. 31 is a lay-flat view of a headgear.

FIG. 32 is a lay-flat view of a headgear.

FIG. 33 is a lay-flat view of a headgear.

FIG. 34 is a lay-flat view of a headgear.

FIG. 35 is a view of the drape configuration of a headgear when held by a patient.

FIGS. 36A and 36B are views of a headgear on a patient's head.

FIG. 37A is a panel for part of a strap.

FIG. 37B shows the panel of FIG. 37A bonded to another strap panel.

FIG. 38 shows another strap panel.

FIGS. 39A and 39B are top and side views respectively of a strap end feature.

FIGS. 40A and 40B are lay-flat views of a headgear with stamped parts.

FIG. 40C is a view of a rear portion for a headgear with stamped parts.

FIG. 41 is a partial view of two configurations of a headgear.

FIG. 42 is a view of a continuous fusing process for forming part of a headgear.

FIG. 43 is a view of another continuous fusing process for forming part of a headgear.

FIG. 44 is a view of another continuous fusing process for forming part of a headgear.

FIG. 45 is a view of another continuous fusing process for forming part of a headgear, where the headgear part is stretched before it is fused.

FIG. 46-1A is a view of a headgear panel and two inserts.

FIG. 46-1B is a view of two fused panels having pockets formed between them.

FIG. 46-2 is a view of two fused panels having voids formed between them.

FIG. 46-3 is a view of two fused panels having a continuous pocket formed between them.

FIG. 47A is a view of a headgear and interface where the fused headgear panels define air conduits.

FIG. 47B is a cross-sectional view of an air conduit through the line A-A of FIG. 47A.

FIGS. 48A-D are views of steps in a process for joining a strap to another portion of a headgear.

FIG. 49A is a view of panel, and FIGS. 49B-D are views of illustrated fusing patterns which may be applied to the panel of FIG. 49A.

FIG. 50 is a view of part of a headgear.

FIG. 51 is a view of part of a headgear.

FIG. 52 is a view of a rear portion of a headgear.

FIG. 53 is a view of another rear portion of a headgear.

FIG. 54 is a view of a rear portion of a headgear when the headgear is worn by a patient.

FIG. 55A is a view of a fusing arrangement provided to a panel and FIGS. 55B and 55C show the performance of the fused panel under different loading conditions.

FIG. 56A is a view of a panel which has been pre-stretched and fused, and FIG. 56B is a view of the fused panel of FIG. 56A when the pre-stretching is released.

FIG. 57 is a view of a headgear and interface when worn by a patient.

FIG. 58 is a view of a headgear and interface when worn by a patient.

FIG. 59 is a view of a headgear and interface.

FIG. 60A is a view of a headgear and interface with an integral adjustment fixture for a strap of the headgear.

FIG. 60B is a partial cross-section through the line A-A of FIG. 60A.

FIG. 61A is a view of a strap with an integral fixture.

FIG. 61B is a view of the fixture and panels forming the strap of FIG. 61A before the components are joined together.

FIG. 62 is a view of another fixture.

FIG. 63 is a view of two lapped panels configured to provide a smoothed edge.

FIG. 64 is another view of two lapped panels configured to provide a smoothed edge.

FIG. 65 is a view of two lapped panels configured to provide smoothed edges to both sides of a panel.

FIG. 66 is a view of two lapped panels configured to provide smoothed edges to both sides of a panel.

FIG. 67 is a view of two lapped panels configured to provide smoothed edges to both sides of a panel.

FIG. 68A is a view of a headgear and an interface.

FIG. 68B is a cross-sectional view through the line A-A of FIG. 68A.

FIGS. 69A and 69B are views of a process for forming a slider feature.

FIG. 69C is a view of the panel with a slider feature of FIG. 69B, with another panel folded about its edges to smooth them.

FIG. 70A is a view of a panel with fused adjustment features formed in it.

FIG. 70B is a cross-sectional view taken along the length of the panel of FIG. 70A.

FIG. 71 is a view of a panel with fused adjustment features formed in it.

FIG. 72A is a view of a headgear and an interface.

FIG. 72B is a cross-sectional view through the line A-A of FIG. 72A.

FIG. 72C is an illustrated view of a continuous process for forming a headgear portion with a filament in it.

FIG. 72D is a view of two fused panels with a filament slidable between them.

FIG. 73 is a view of a headgear which is adjustable by drawing on filaments provided within portions of the headgear.

FIG. 74 is a lay-flat view of a headgear.

FIG. 75 is a lay-flat view of a headgear.

FIG. 76 is a lay-flat view of a headgear.

FIG. 77A is a lay-flat view of a headgear.

FIG. 77B is a partial view of a headgear such as the headgear of FIG. 77A, illustrating the connection of a strap to the rear portion of the headgear.

FIG. 78 is a lay-flat view of a headgear.

FIG. 79A is a lay-flat view of a top strap for a headgear.

FIG. 79B is a lay-flat view of a headgear including the top strap of FIG. 79A.

FIG. 80 is a lay-flat view of a headgear.

FIG. 81 is a lay-flat view of a headgear.

FIG. 82A is a partial pre-assembly view of two straps and a rear portion of a headgear with an overmolding component.

FIG. 82B is a lay-flat view of a headgear.

FIG. 83A is a partial pre-assembly view of a strap and rear portion of a headgear.

FIG. 838 is an assembled view of the strap and rear portion of FIG. 83A.

### DETAILED DESCRIPTION OF PREFERRED EMBODIMENTS

Described herein are various embodiments of a headgear for a respiratory interface. Such a headgear may include a plurality of panels, configured to present the headgear with at least one lapped region in which at least two of the plurality of panels overlap and underlap each other, and at least one non-lapped region in which one or more of the panels are not lapped by another of the panels. The panels at one or more of the lapped regions may be joined together, such as by fusing.

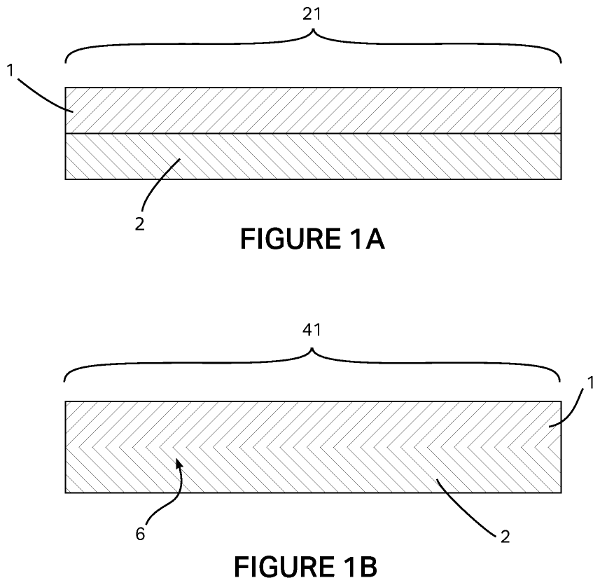

Details of example lapped and non-lapped panel configurations and fusing of those panels will now be described with particular reference to FIGS. 1A to 5C. These figures may be taken to illustrate cross-sectional views or partial cross-sectional views of a headgear according to the disclosure, or of various component parts from which a headgear may be built up.

FIG. 1A is a sectional view of two lapped panels, a first panel 1 and a second panel 2.

The panels may be selected from any suitable panel-like material. This may include textiles being networks of natural and/or artificial fibres, and more particularly fabrics as may be made by weaving, knitting, spreading, crocheting, bonding, or other available methods.

The panels may also include any other non-conventionally textile materials which may be provided in a panel-like form, such as a plastic or composites.

The individual panels may according to at least some configurations be single plies or single layers of one material or a unitary composite of materials, as opposed to multiple layers of the same or different materials.

The panels may be of a sheet-like form or may comprise tubular panels.

The panels may have a 2D shape. Alternatively, one or more of the panels which form the headgear may have a 3D shape.

In at least some embodiments, some or all of the plurality of panels may be of flexible materials, and particularly materials which may drape under their own weight. Such materials may be particularly suited to forming parts of a headgear which is to conform to the shape of a patient's head.

Each panel of the plurality of panels that comprise the headgear defines two opposed major faces. In at least some embodiments it may be these major faces which are to be lapped against each other and joined together within the one or more lapped regions of the headgear.

The plurality of panels may each be panels of the same material.

In various forms at least some of the panels may be of different materials.

Different panels, such as may include different materials, may define various material properties or characteristics. The headgear which is formed from these panels may in turn be defined by these material properties. Where a panel is treated by being fused or lapped panels are fused together, the properties of the respective panels and the headgear may further be altered by the fusing.

The material properties of a panel include its stretch properties. The stretch properties of a panel, or the headgear, or a part of the headgear, refer first to its in-plane extensibility. A panel having a relatively greater stretchability has a relatively greater extensibility, and vice-versa.

The in-plane stiffness of a panel refers to the degree of resistance to in-plane extension or stretch of the panel. Conversely, the flexibility of a panel refers to an out-of-plane stiffness property of a panel, for example the degree to which a panel will drape.

The extensibility of a panel may be omnidirectional, such that the panel or headgear may have the same in-plane extensibility in all directions. The stretch properties of a panel may differ directionally, so the panel has either different extensibilities or is extensible and inextensible in different in-plane directions.

A stretch panel may be one that has at least some degree of in-plane extensibility. A non-stretch panel may be relatively inextensible in-plane in at least one direction.

The panels may have differing stretch properties from each other. For example, a first panel may have a first stretchability, while a second panel has a second different stretchability.

Where the panels are lapped against each other but not fused together they may define another third stretchability. Where the panels are lapped and fused they may define another fourth stretchability. Still further, either the first panel or second panel are not lapped but are fused it may define a fifth stretchability, which is different to the respective one of the first stretchability or second stretchability.

A panel which has stretch properties such that it is extensible may or may not, when a stretching load is removed, allow recovery of some or all of the extension. In at least some configurations the stretch properties of a stretch panel may include recoverability, so that a panel may be stretched then when the stretching load is removed it may return to or towards its original unstretched shape.

Recoverability may for example be achieved by elasticating a panel. An elasticated panel includes at least one elastomeric, rubber, or rubberised component. For example, an elasticated panel may include at least some fibres of such an elastic material. Conversely, a non-elasticated panel is a panel which does not include any such an elastomeric, rubber, or rubberised component. An elasticated panel has greater elasticity, due to the at least one elasticated component, than a non-elasticated panel of the same material but without the at least one elasticated component.

The properties of a panel may include the texture of the panel at one or both of its major faces, or at one or more of the edges bounding the two major faces.

Another property of a panel may be the softness or hardness of one or both of the major faces or one or more of the edges bounding the two major faces of the panel.

Further properties which one or more of the panels may have include different densities, surface hardnesses, Young's moduli, thicknesses, colours at one or both of the major faces or bounding edges, coefficients of friction at one or both of the major faces relative to a reference material. The different material properties may also include different degrees of breathability, different degrees of hydrophobic or hydroscopic qualities, different permeability, or different transparencies or sheers.

In addition to having any of such different material properties between panels, an individual panel itself may have one or more of such characteristics which are directionally or locationally different within the panel itself. For example, as previously described, a panel may have a directionally different stretch properties. A panel may also have directionally different texture or softness, flexibility, or frictional coefficients.

In addition to different material properties, different panels of the plurality of panels may have different physical configurations, including both thickness and in-plane dimensions.

Another example of a characteristic of a textile panel may be whether it comprises a cut or uncut pile. Whether the pile is cut or uncut may provide different surface characteristics. For example, an uncut pile may present loops of material at a panel surface. Such a material with an uncut pile may generally be known as an unbroken loop (UBL) material.

The presence of such loops may be desirable to act as the loop portion of a hook and loop fastener system. By this configuration a hook and loop fastener system may be provided without having to attach any additional loop-providing component to the panel. This may go at least some way to providing a headgear which is of reduced thickness.

One or more panels to be utilised in the headgear may be unravelable, meaning that their constituent fibres may be unravelled.

In addition or alternatively, one or more panels to be utilised in the headgear may not be able to be unravelled, what may be known as a free-cut material. Such a material may be cut and may not or may at least be resistant to unravelling of the constituent fibres along the cut edge. Advantageously, such materials can be cut to shape with the cut edge of the panel forming an edge of the headgear without the need for further processing.

The panels may be selected of materials having any other material properties or panel configurations such as may provide for the desired functionality of a headgear which is to comprise the panels.

In addition to different material properties and characteristics provided by combinations of different individual panels, composite characteristics may be provided at a lapped region where the panels are lapped.

For example, the respective panel thicknesses will form a lapped region with a thickness equal to the sum of the respective panels.

In another example, one panel at a lapped region is extensible in one direction, and another panel at the lapped region is extensible in another direction which is different or potentially perpendicular to the direction of the former panel. Particularly where the panels are extensible only in directions which are perpendicular or substantially perpendicular to each other, a functionality may be provided where the panels at the non-lapped regions may stretch in their respective directions, but at the lapped region may be substantially inextensible.

It will be appreciated that numerous other such combinations of panels having one or more different material properties may be configured so as to have desired properties both at non-lapped regions and lapped regions.

In addition to the use of panels having one or more different material properties, the properties of the headgear may be at least in part determined by different configurations of respectively lapped panels at the one or more lapped regions of the headgear.

Lapped regions of the headgear may be fully or only partially joined together. For example, a lapped region may include both joined regions where the adjacent panel surfaces have been joined together, and non-joined regions where the adjacent panel surfaces are not joined together.

Panels which are joined together may be directly joined to each other. A direct join between two panels is a join of the two panels to each other, without any other material between the two panels. For example, a direct join between two panels would exclude any interposed adhesive or an interposed intermediate layer. A direct join may be a join of one or both of the materials of the respective panels to each other.

According to various embodiments, at least part of a lapped region of the headgear may have its panels joined by being fused.

FIG. 1B shows the first panel 1 and second panel 2 of FIG. 1A which have been joined together by being fused.

Fusing defines a melting of a panel or at least a constituent material of the panel. In relation to fusing together of two panels, fusing refers to the melting of one or both of the panels into the other or each other, respectively. Thus, two panels may be fused by either a) melting only one or primarily one panel into the other, or b) a simultaneous melting of both panels into each other.

Accordingly, a panel which is to be fused should include a meltable material, such as an artificial fibre. For example, a polymer. Similarly, where two panels which are to be fused together at least one of the two panels should include a meltable material, such as an artificial fibre.

As seen in FIG. 1B the lapped region 21 of the first panel 1 and second panel 2 of FIG. 1A have been fused into each other, or one has been fused into the other at their lapped faces, so that they are integral with each other and define a fused panel 6.

In one form, the fusing of a panel or panels may be non-additive, in that the fusion involves a treatment applied to the panel or panels and does not involve the use of any additional material such as an adhesive interposed between two panels to fuse them together.

Fusing may involve one or both of the application of heat and pressure to the panel or panels.

Fusing may generally be by a welding.

Where panels are fused together by welding, one or more of the panels is of a weldable material.

Welding may include forms of plastic welding.

For example, fusing may be provided by radio frequency (RF) or high frequency (HF) welding, where the material to be fused comprises a dipolar material which is heated and melted by electromagnetic excitation. Examples of materials which may be fused by radio frequency or high frequency welding include PVC, CPVC, Polyurethane, EVA, PVDC, PET, and nylons. Fusing by radio frequency or high frequency welding may be provided to materials which at least partially include a dipolar material.

Further examples of materials which may be fused by radio frequency or high frequency welding include PETG, TPU, and LDPE.

One or more panels which are to be fused by welding, including by radio frequency or high frequency welding, may thus comprise or consist of a dipolar material, which are weldable by these processes. When the fusing process, such as welding by a radio frequency or high frequency welding, is applied to the panel or panels the panel or panels respectively may generally be caused to consolidate or solidify.

A panel or panels may be fused to varying degrees. For example, in the case of radio frequency or high frequency welding, one or more of the welding platen separation, weld energy, and welding time may be varied to increase or decrease the degree of the fusing.

Relatively lower degrees of fusing may cause partial or localised consolidation of the fused material. Relatively greater degrees of fusing may cause more complete or generalised consolidation of the fused material. A material which is exposed to a sufficient degree of fusing may cause the original material, for example a dipolar fabric, to form a solid plastic where it is fused.

Where the panels are to be fused by high frequency welding the panels specifically include nylon, and in particular a nylon content of about or more than 80%. A remaining portion of the panel may include spandex or other similar polyether-polyurea copolymers. Particularly where the panels are fabrics which are fused by high frequency welding, the fabrics may have a density of about 160 g/m² or greater.

Other examples of applicable welding methods to fuse a panel or panels include ultrasonic welding, vibration or friction welding, hot wedge welding, hot air welding, and induction welding.

The fusing of two panels is at least primarily to be a fusing of the lapped faces of the respective panels.

Panels joined together by fusion may have different degrees to which they are fused together. This degree of fusion may be defined by peel forces between the fused surfaces; the force necessary to peel the two fused surfaces apart, given some reference peeling angle.

Panels joined together by fusion may define a fused zone, at which the peel forces required to peel the panels apart are non-zero. In particular, the peel forces at a fused zone may be significant and may even be such that the panels cannot be peeled apart without destruction of the respective panels.

Panels joined by fusion may further define a non-fused zone, at which the peel forces to separate the panel are zero or substantially zero.

In addition to fused zones and non-fused zones, panels joined by fusion may define one or more transition zones, at which the peel forces transition between those of the fused zone and the non-fused zone. A transition zone may define a gradient of peel forces between that of the fused zone and the non-fused zone.

A transition zone may be defined by peel forces across its whole area which are between that of an adjacent fused zone and an adjacent non-fused zone. A transition zone may additionally or alternatively be defined by a decrease in density of fused area relative to an adjacent fused zone, and thus a relatively lesser average peel force required to separate the two panels than at the adjacent fused zone.

One or more lapped regions may be fully fused, so an entirety or substantial entirety of the lapped surfaces are joined together. For example, the whole of the lapped surfaces may be joined together except for some minor parts such as may for example be used in providing a desired texture or surface finish to the headgear. A substantial entirety of the lapped surfaces may be about 90% of the lapped area, or even about 95% of the lapped area.

In addition or alternatively, one or more lapped regions may be partially fused, defining both fused zones and non-fused zones within the lapped region.

In addition to or alternatively to being characterised as entirely or substantially entirely fused, the fused configuration of panels of a headgear according to the disclosure may be described by a straight-line condition. According to the straight-line condition, a straight line is drawn between any two edges of a lapped region of the headgear, or portion of the headgear such as a rear portion. Along the defined line the length that is within fused areas is greater than the length within non-fused areas. In other words, along the straight line there is more welded length than there is non-welded length.

The welded length of a line that satisfies the straight-line condition may be anywhere between a majority of the total line length to the entirety of the line length.

The straight-line condition may be satisfied between two given points, such as between an upper edge of the lapped region and a lower edge of the lapped region of a rear portion of the headgear.

The straight-line condition may be satisfied between more than two pairs of given points, such as between a plurality of respective upper and lower edges of a lapped region of the rear portion of the headgear.

The straight-line condition may be satisfied along an entire width of the rear portion of the headgear, between successive locations along the upper and lower edges of a lapped region of the rear portion of the headgear.

The straight-line condition may be satisfied along any straight line that can be drawn between two points located on an edge or edges of a given lapped region of the rear portion of the headgear.

Along a line that satisfies the straight-line condition the fused and non-fused lengths may be arranged in particular ways. For example, at the ends of the lines adjacent to each respective edge the lapped panels may be fused. In this configuration any non-fused portions along the line are provided along a central portion or portions of the line, away from the line ends.

In some configurations along a line that satisfies the straight-line condition non-fused portions along the line are only located away from the ends of the line.

Where a line between two edges of a lapped portion satisfies the straight-line test and fused portions are located at both ends of the line, a cumulative length along the line of the end portions where the lapped portion is fused may be greater than a cumulative length of any central non-fused portions along the line.

While generally described in relation to the rear portion of the headgear, other parts of the headgear such as one or more straps of the headgear may also satisfy the straight-line condition at one or more locations along their length or along their entire lengths.

The fusing of a panel or multiple panels may change one or more properties of the panel or panels.

For example, melting and re-solidification of a material of the panel may cause the panel to be one or more of thinner, denser, more stiff, less stretchable, less recoverable, or of a greater yield strength in stretching. Accordingly, in addition to joining panels together, selectively fusing the headgear at different lapped and non-lapped regions may allow control of the performance of the headgear.

For example, fusing of particular parts of the headgear may be utilised to create load transfer paths between different parts of the headgear.

Conversely, parts of the headgear which are not fused may be located to define stretch zones, or areas of relatively greater thickness or suppleness.

The fusing of a panel or panels may also change one or more surface characteristics of the panel. For example, where a panel is of an unbroken loop material at a surface, the melting and re-solidification the panel or a constituent material of it may reduce an number of unbroken loops presented at the surface of the panel. This may reduce the effectiveness of the panel in coupling with the hooks of a hook and loop fastener. It may even act to prevent the hooks of a hook and loop fastener system from being able to couple with the panel.

Accordingly, the selective fusing and non-fusing of parts of panels may provide parts of the same panel or panels which have an unbroken loop surface and others which do not.

In addition to providing a desired performance of the headgear, selective fusing—both of the parts of a panel or panels which are fused, a degree to which they are fused, and the shape or orientation of the fused parts of the panels—may be utilised to provide different properties to the panels to change the ‘feel’ or appearance of the headgear. This may be employed to provide cues to the patient or other user as to orientation or use of the headgear.

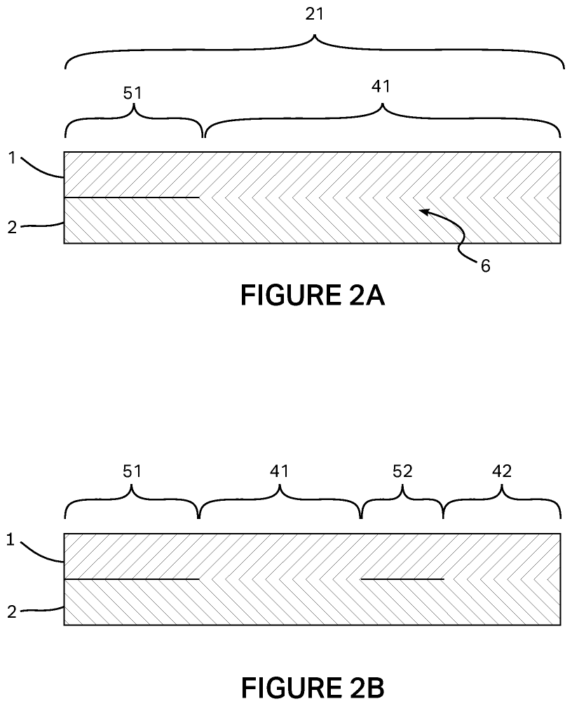

FIG. 2A shows a first panel 1 and second panel 2 lapped with each other to define a lapped region 21, which have been partially fused together.

A majority of the lapped region 21 has been fused to define a fused zone 41, while a remainder of the lapped region is left unfused and defines a non-fused zone 51. At the non-fused zone 51 the panels 1 and 2 are not joined to each other by being fused and have zero or substantially zero peel force required to separate the layers at this zone.

FIG. 2B shows the panel layup of FIG. 2A, but where it has a second non-fused zone 52 which separates the fused zone 41 of FIG. 2A into a first fused zone 41 and a second fused zone 42.

The panels may be fused and left unfused at any configuration of different areas to provide the desired characteristics of the headgear.

The fusing of the panels, for example where one or both of the panels are melted or partially melted into each other by welding, may result in the thinning of the panel layup at fused zones. This may particularly be the case where the fusing is provided by the application of pressure at the parts of the panels which are to be fused.

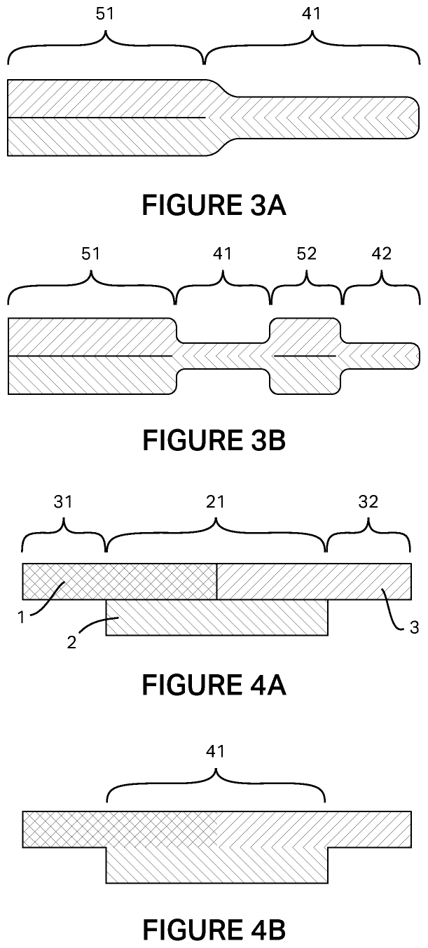

FIG. 3A illustrates a layup of a first panel 1 and second panel 2 which have been partially fused to define a non-fused zone 51 and a fused zone 41. At the fused zone the thickness of the layup is less than that of the panels at the non-fused zone.

FIG. 3B shows the same layup as in FIG. 3A, but where there are two spaced apart fused zones 41 and 42, and two non-fused zones 51 and 52. The first non-fused zone 51 is at an end of the panels. The second non-fused zone 52 is located between the two fused zones 41 and 42.

FIGS. 1-3 have illustrated configurations where two panels are fully lapped with each other. Panels also may be arranged in other configurations, such as where one or both panels define a non-lapped region, or where panels are butted against each other edge to edge. FIG. 4A shows a layup of a first panel 1, second panel 2, and third panel 3 which illustrates some such configurations.

In FIG. 4A the first panel 1 and second panel 2 are each partially lapped with each other. The third panel 3 then partially laps the second panel 2, and the edges of the first panel 1 and third panel 3 abut each other.

The arrangement of FIG. 4A defines a first non-lapped region 31, a lapped region 21, and a second non-lapped region 32.

FIG. 4B shows the arrangement of FIG. 4A where the panels are fused together. As seen in FIG. 4B the lapped surfaces of the first panel 1 and second panel 2, and the second panel 2 and third panel 3 have been fused together.

Also seen in FIG. 4B the abutting ends of the first panel 1 and third panel 3 have been fused together.

The characteristics of the headgear at either a lapped region or a non-lapped region also be influenced by whether or not, and the degree to which, the panel or panels are fused.

For example, the fusing of a panel by its melting may result in one or more of a localised thinning, reduced extensibility, or reduced flexibility at and potentially also adjacent to the fused zone.

Accordingly, fused zones of the headgear may be located at both non-lapped regions and lapped regions to provide desired properties to the headgear.

One or more of the panels at a non-lapped region of the headgear may also be fused. Instead of acting to join together two panels, fusing at a non-lapped region may function to alter the material properties of the panel.

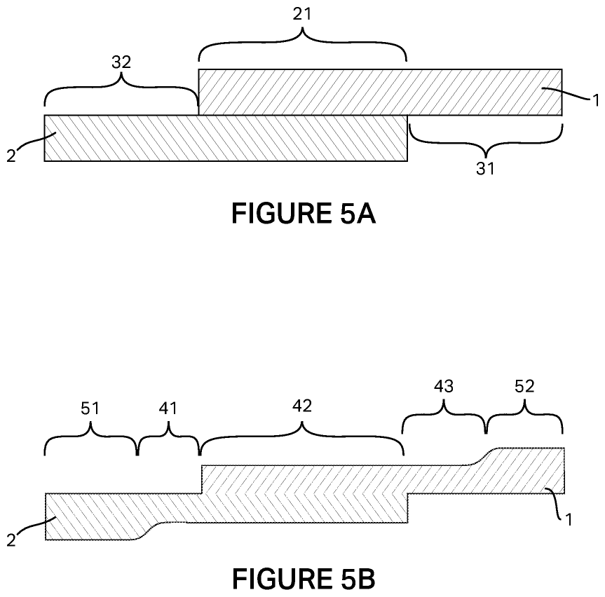

FIG. 5A shows a first panel 1 and second panel 2 which are partially lapped with each other to define a first non-lapped region 31 of the first panel, a lapped region 21, and a second non-lapped region 32 of the second panel.

FIG. 5B illustrates the result of a fusing applied to both the lapped region 21 and part of both of the first non-lapped region 31 and second non-lapped region 32. In the illustration of FIG. 5B the fusing is such that it results in a thinning of the panel or panels where it is applied.

As seen in FIG. 5B the fusing causes the panels to have a first fused zone 41 of the first panel, a second fused zone 42 of the fused lapped first panel 1 and second panel 2, and a third fused zone 43 of the fused second panel 2. The remaining lateral portions of each of the first panel and second panel are not fused and define the first non-fused zone 51 and second non-fused zone 52.

Panels may be singly lapped, as seen in any of FIGS. 1A-4B where a panel is only either overlapped or underlapped by one other panel. When single lapped panels are joined together they form a single lapped joint. Panels may also be double lapped, so that a panel is both underlapped and overlapped on respective major faces of the panel by two other panels. Where double lapped panels are joined together may be referred to as a double lapped joint.

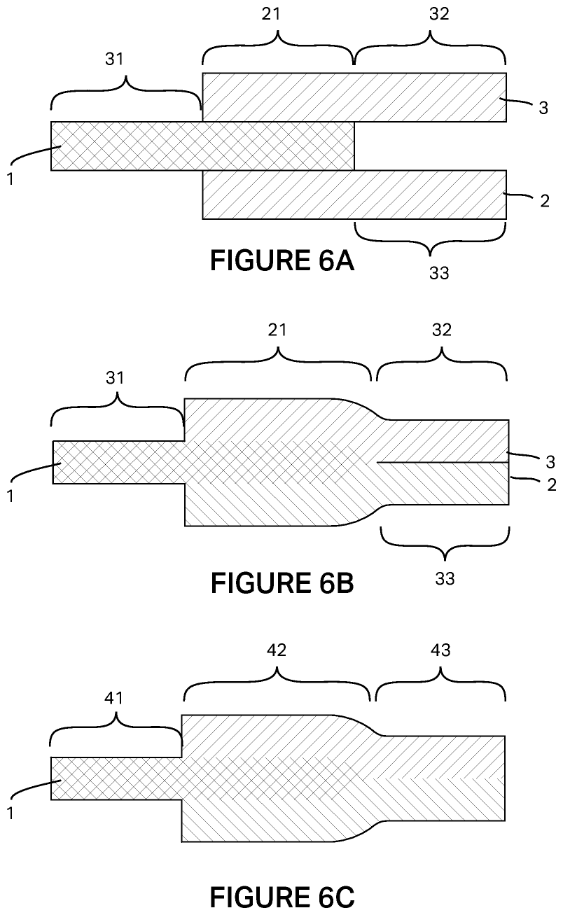

FIG. 6A shows a layup with a first panel which is both overlapped and underlapped, or double lapped, by, respectively, a second panel 2 and third panel 3. The layup defines a first non-lapped region 31, a lapped region 21 at which the first panel is lapped on either side by the second panel and the third panel, and a second and third non-lapped regions 32 and 33 of each of the second panel 2 and third panel 3 respectively.

FIG. 6B shows a first fused arrangement of the layup of FIG. 6A, where the lapped region 21 is fused. The fusing of the lapped region 21 joins both the lapped surfaces of, respectively, the first panel 1 and second panel 2, and first panel 1 and third panel 3, together.

As seen in FIG. 6B each of the non-lapped regions 31-33 remain unfused. At the non-lapped regions 32 and 33 the second and third panels are shown to now be lapped with each other due to the fusing of the adjacent lapped region.

FIG. 6C shows another fused arrangement of the layup of FIG. 6A, where the fusing has been applied across the whole section to define a first fused zone 41 of the first panel, a second fused zone 42 of the lapped region 21, and a third fused zone 43 of the second and third panels, which are now fused together.

In comparison to a single lapped joint, a double lapped joint such as shown in FIG. 6B or 6C may have the advantage of reducing the potential for inducing torsion in the joint under lateral loading of the respective panels. Such torsion may cause twisting or other deformation of the joint, so that at least part of the headgear does not lay flat. This may be undesirable in some circumstances, such as where the headgear is to lie flat against a patient's head, because any such torsion of the headgear may result in points of increased pressure against the patient's head, and thus discomfort.

Reduced or eliminated torsion at an interface between panels may also increase the strength of the headgear where the panels are joined or bonded together. Torsion of the panels at a joint may cause them to be subjected to peel stresses when the panels are under tension. Joints may have relatively limited strength under such peel stresses, where the panels are pulled away from each other perpendicularly to the joined surfaces. The reduction of torsion may mean that the panels are not twisted and exposed to peel stresses, but rather to predominantly or only to shear stresses. Some bonds, particularly fused or welded bonds, may be able to provide greater strength under shear stresses than peel stresses. This means that the strength of the headgear may be increased by reducing torsion at lapped panels.

In other configurations, for example where loads are not such as to cause significant torsion in the joint, a single lapped joint may be preferable to a double lapped joint as it may provide a joint of lesser thickness.

Single lapped joints of the arrangement of FIGS. 4A and 4B may also function similarly to a double lapped joint in reducing torsion, and thus potentially increasing the strength of the joint, where lateral loads are to be applied to the first panel 1 and third panel 3.

While the foregoing has described various example layups of different panels, and different fused and non-fused configurations of such panels, it will be appreciated that any number of variations or combinations of such layups and their fused configurations may be provided to form a headgear.

While shown comprising various panels of illustrative thicknesses, it will be appreciated that the foregoing layups may be provided with panels of different thicknesses and combinations of thicknesses, among any other desired material properties.

Unless the context indicates otherwise, the periphery of panel, portion, region, or the headgear itself may generally be understood as referring to the perimeter and more particularly a border within the perimeter, of the respective panel, portion, region, or headgear. Where the context requires, this may otherwise be referred to as an internal periphery.

Conversely, an external periphery of a panel, portion, region, or the headgear itself will generally be understood as referring to a border outside of the perimeter of the respective panel, portion, region, or headgear.

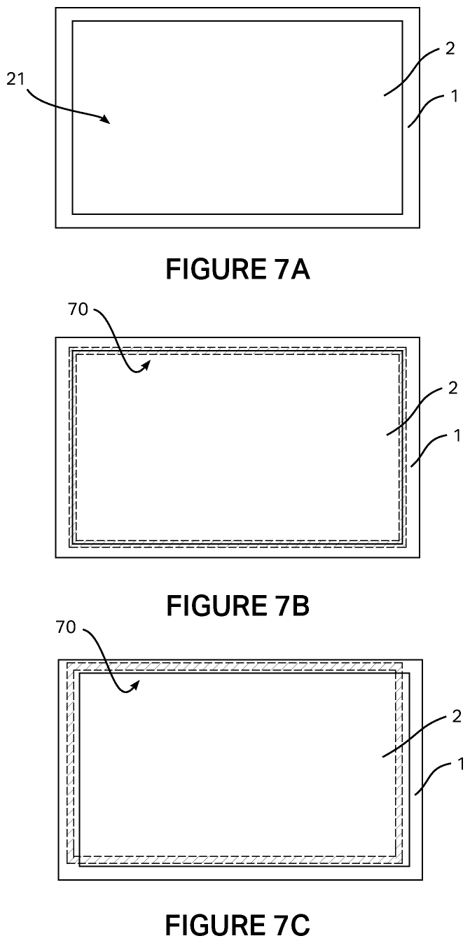

FIGS. 7A-7C show plan views of two panels which are to form part of a headgear.

As seen in FIG. 7A a first panel 1 provided under a second panel 2, which is fully lapped by the first panel. The panels define a lapped region 21, which is of the same size as the second panel 2. The panels are to be joined together to form part of a headgear. They may be joined by fusing or specifically by welding, as has been described. They may additionally or alternatively be joined at least in part by any number of methods, such as a stitching or by the use of an adhesive. For the purposes of this example the panels are to be joined by being fused together, and particularly by welding.

FIG. 7B shows a shaded boundary representing the weld 70 about the perimeter of the lapped region where the panels 1 and 2 of FIG. 7A have been joined.

The arrangement of FIG. 7B illustrates a conventional method of joining two or more panels together, particularly by welding and where a foam layer is interposed between the two panels and it is desirable to minimise an amount of the foam layer that is welded.

However, welding the panels about the perimeter of the lapped region 70 may present difficulties due to the narrow nature of the weld and the fine tolerances it necessitates. If one or both of the panels are not placed in their correct locations relative to the weld tool, or if the tool is misaligned relative the panels, the weld may not end up in its desired location.

FIG. 7C shows the arrangement of FIG. 7B, but where the weld 70 is misplaced relative to the panels 1 and 2. As a result, the weld 70 is located partially outside of the bounds of the second panel 2 (at the left-hand side and top of the second panel), and partially only within the bounds of the second panel (at the bottom and right-hand side of the second panel). This may mean that the panels are not suitably joined together.

While illustrated in FIG. 7C as being a misalignment between the panels and the weld tool, it will be appreciated that misalignments may additionally or alternatively be introduced by the locations of the panels relative to each other. Furthermore, while the misalignment in FIG. 7C is illustrated as a translational misalignment it will be appreciated that there may additionally or alternatively be rotational misalignments.

The issues associated with misalignments may be minimised if the width of the weld 70 is increased, such that expected degrees of misalignment will not cause the join to be located outside of the periphery of the second panel 2. The issues may also be minimised if the weld 70 provided to extend past the lapped region of the panels, onto where in a correct alignment only the first panel 1 will be. However, such changes may be contrary to the conventional teaching as they involve increasing rather than minimising the area of the panels which are subjected to welding.

Even if the width of the weld is increased and the weld is located to extend beyond the lapped region, misalignments may reduce the total amount of joined area the panels and thus the strength of their join.

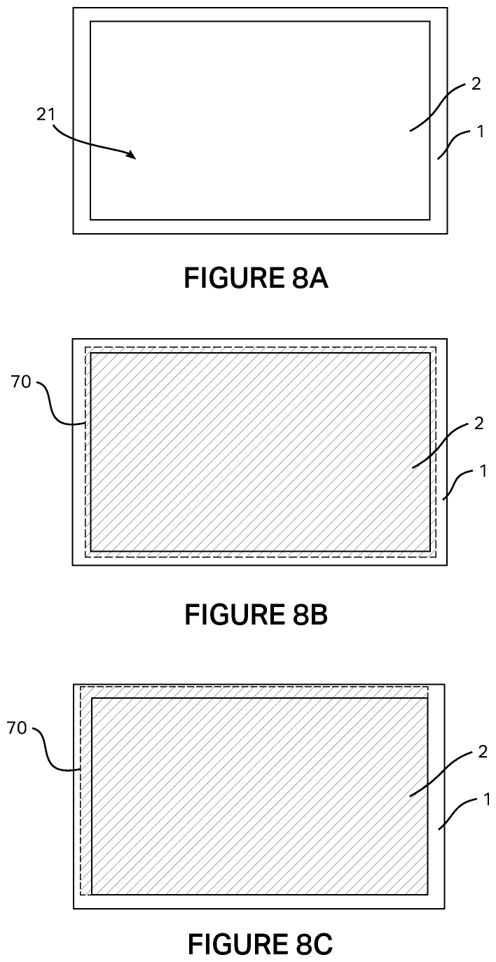

A further method of avoiding join misalignment is illustrated in FIGS. 8A-8C. FIG. 8A shows the same arrangement as FIG. 7A, with a first panel 1 fully lapping a second panel 2.

FIG. 8B shows the same panels where the weld 70 is applied across an entirety of the lapped region 21, and also extends beyond the lapped region onto the non-lapped region of the first panel 1.

FIG. 8C shows the arrangement of FIG. 8B, but where the weld and panels are misaligned from each other. Because the weld 70 is larger than the second panel 2, the misalignment can be compensated for and the entire lapped region 21 is still welded. Additionally, because the weld 70 covers the entire area of the second panel 2 rather than just a boundary about the internal and external periphery of it, the effect of any misalignment on the total welded are of the lapped region may be reduced.

Accordingly, it may be desirable to increase or even maximise the welded area of the headgear.

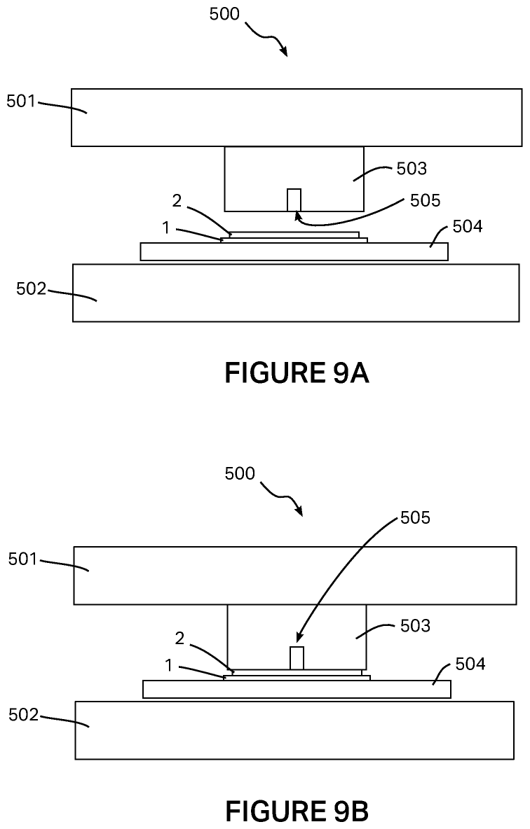

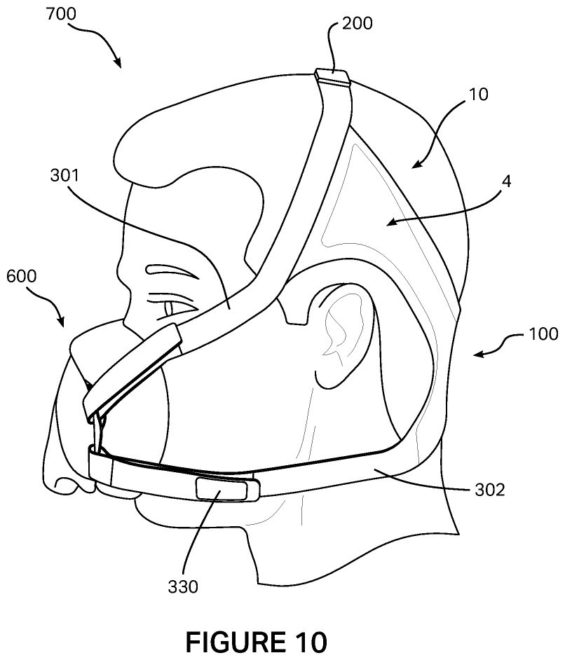

FIGS. 9A and 9B illustrate a welding apparatus 500, such as a high frequency welder, for use in welding the panels of a headgear.

The welding apparatus 500 includes top and bottom welding platens 501 and 502. Within the platens are an upper die 503 and lower die 504. The platens are to be movable away from each other to open the dies and together to bring the dies into proximity for welding.

Panels such as a first panel 1 and second panel 2 are located between the two dies.

The upper die and lower die each define die faces 506. These faces are brought into proximity with each other across the panels in order to provide the fusing of the panels.

According to a type of welding utilised, the die faces will variously interact to provide the fusing of the panel material therebetween. For example, in the case of a direct thermal welding one or both of the dies may be heated to melt the panels which are pressed between the dies. Or, in the case of an ultrasonic welding, the vibration of the dies at the die faces which contact the panels will heat the panels and provide the fusing of the panels.

In the example of high frequency welding the electromagnetic field provided across the dies is of sufficient strength between the two die faces 506 to excite and melt a dipolar material located between the die faces.

While one or both of the dies may be heated for direct thermal welding, one or both of the dies may also be heated where other types of welding are used, such as ultrasonic welding or high frequency welding.

Where one or both dies are heated they may for example be heated to about 100° C.

Such heating of one or both of the dies may increase the temperature of the panels before they are fused. This may increase the effectiveness of the fusing operation.

One or both of the upper die 503 and lower die 504 may include one or more recesses of the die away from the die face 506. As seen in FIGS. 9A and 9B the upper die 503 includes a recessed region 505. The recess creates a relatively increased distance between the two dies at the recessed region 505. This increased distance may reduce a degree of welding of the materials within the footprint of the recessed region 505. It may even cause the materials within the footprint of the recessed region to remain unwelded or at least substantially unwelded.

For example, in the case of direct thermal welding, the upper die 503 will not contact the panels at the recessed region 505 and may accordingly not cause the panels to be melted at the recessed region. A similar functionality would apply in the case of ultrasonic welding.

Where the panels are to be fused by high frequency welding the recess 505 will create a region of reduced electromagnetic field strength and consequently a reduced heating of the panels at the recess 505. The recess 505 may be of a sufficient depth as to prevent a melting of one or more materials of the panels at the recess 505.

The power supplied to the high frequency welding apparatus and the separation between parts of the two dies may thus be used to control where the panels are to be fused, and to what degree they are to be fused.

Regions of the dies which are closest to each other during welding may cause the parts of the panels between them to be fused, while parts of panels between less proximate parts of the two dies may be fused to a lesser degree or remain unfused.

Accordingly, design of one or both dies to design the shape of the die face 506 and location and depth of any recessed regions 505 may allow for control of what parts of the headgear are fused and to what degree they are fused, and what parts of the headgear remain unfused.

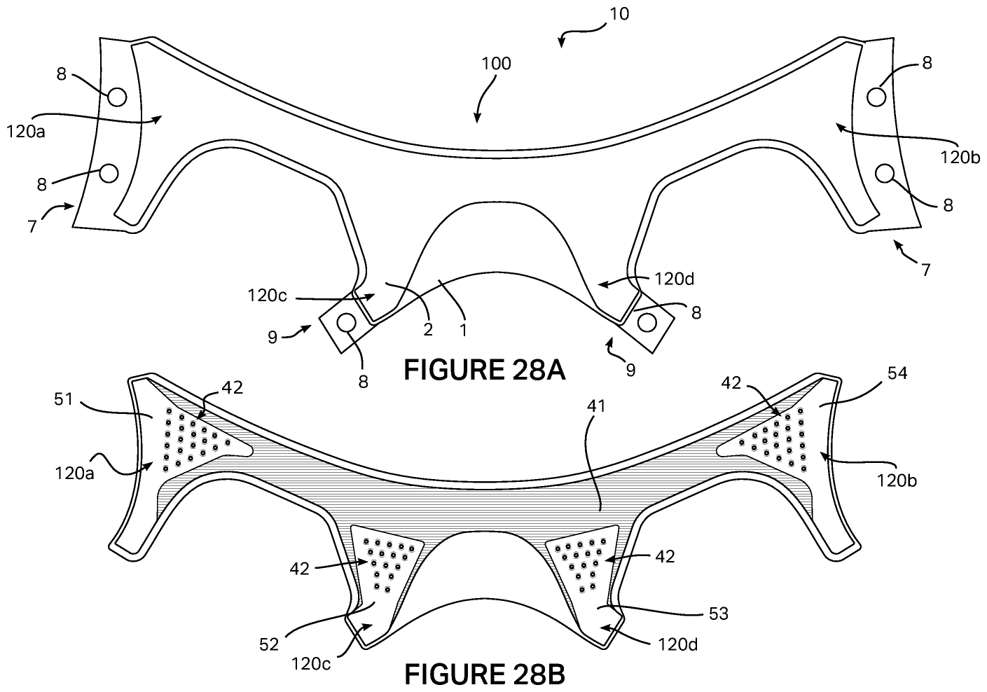

The panels of the headgear are to be placed in the welding apparatus 500 between the upper die 503 and lower die 504. One or more of the panels may include alignment features 8 as will be described in relation to FIGS. 28A and 28B. The alignment features may interface with corresponding features of one or both of the dies to retain the panel or panels in their desired location relative to the dies.

Once the panels are in their desired location the upper die 503 and lower die 504 may be brought together and the welding of the panels commenced.

FIG. 9B shows the welding apparatus 500 where the platens 501 and 502 and dies 503 and 504 have been brought together to weld the panels 1 and 2.

The dies may be brought into a predetermined proximity of the panel or panels. In some configurations the dies may apply pressure to the panel or panels, or at least to regions of the panels which are to be welded together.

The fusing of the panels may be provided in 2D as illustrated in FIGS. 9A and 9B, where the panels are laid flat or substantially flat between the two dies.

The panels or parts of the panels may be welded in a 3D shape. For example, the dies may have complementary 3D shapes, so that the panels drape in a desired way on the dies. The welding may then be applied to the panels when draped in their 3D shape. This may facilitate the forming of a headgear which has a 3D shape.

While not shown in FIGS. 9A and 9B, one or more layers of a non-stick material such as Teflon® may be provided between the panels and one or both dies to prevent sticking of the panels to the die or dies.

While described using the example of high frequency welding, it will be appreciated that the panels may be fused together as described by any number of other methods of fusing and particularly plastic welding including ultrasonic welding, vibration or friction welding, hot wedge welding, hot air welding, or induction welding.

FIG. 10 illustrates a patient 700 wearing a headgear 10 to retain a patient interface 600 to the patient's face. The headgear 10 has an external surface 4 which in use faces away from the patient, and an internal surface 5 (not visible in FIG. 10) which in use faces and contacts the patient.

The external and internal surfaces of the headgear may be the same or different to each other. The internal and external surfaces of the headgear may be defined by panels having either the same or different properties. For example, the surfaces maybe coloured or textured differently to each other to aid in signalling orientation of the headgear to the patient.

As seen in FIG. 10 the headgear 10 has a rear portion 100 located at a back of the patient's head. Two side straps, an upper side strap 301 and a lower side strap 302 are shown. The side straps connect the headgear to the patient interface 600. As seen in FIG. 10 the headgear also has a crown strap or top strap 200 which passes over the head of the patient.

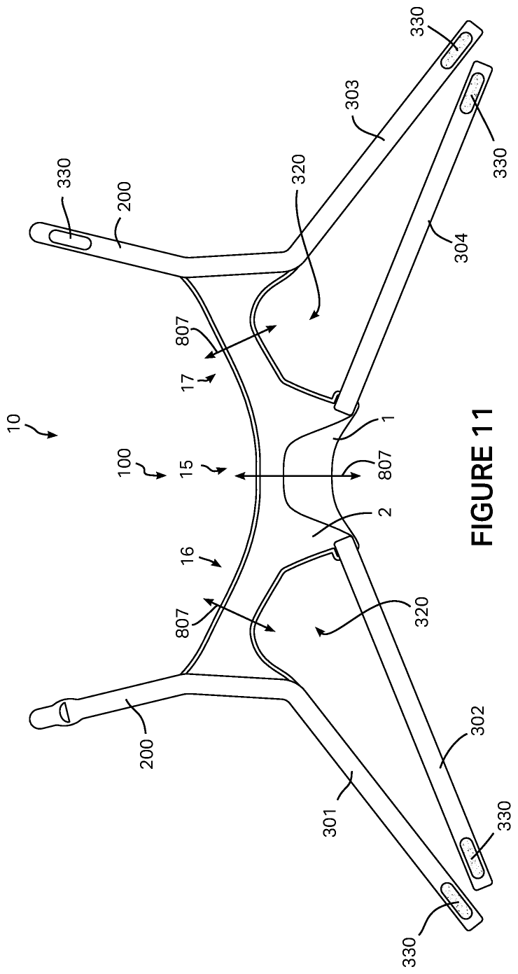

A corresponding two side straps, an upper side strap 303 and a lower side strap 304, may connect to the hidden side of the patient interface 600.

Where there are sets of side straps on each side, a region between the two side straps may define an ear loop 320, illustrated in FIG. 11. The ear loop 320 comprises a portion of the headgear which extends between each respective upper side strap and lower side strap on one side of the headgear. Each upper side strap 301, 303 passes above the ear, and each lower side strap 302, 304 passes beneath the ear. The ear loop 320 is located at least behind the ear. The edge of the ear loop between each upper strap and lower strap may be curved or may comprise a plurality of linear edges arranged about a nominal curve.

It will be appreciated that the straps may be provided in sufficient lengths either shorter or longer than those shown in FIGS. 10 and 11 to reach and couple with an interface, given a range of different patient head sizes.

In other embodiments the headgear 10 may include only two side straps, one strap for connecting to each side of the interface.

FIG. 11 shows a lay-flat view of an embodiment of a headgear 10.

The headgear 10 has a central region 15 and two lateral sides 16 and 17. The central region 15 is for location at the rear middle part of the patient's head, while the two lateral sides 16 and 17 extend use around the patient's head towards respective sides of a patient interface.

As shown, the lateral sides 16 and 17 are be shaped to pass about the ears of a patient.

A height of the headgear, taken in a direction perpendicular to a lateral direction around the head of the patient and shown by the lines 807 of FIG. 11, is larger at a middle of the central region 15 than at part of each of the lateral extensions 16 and 17. In particular, as seen in FIG. 11, the lateral extensions 16 and 17 narrow to approximately 30% to 50% of the height of the headgear at a middle of the central region 15.

As seen in FIG. 11 the height of the second panel 2 at the middle of the central region 15 and at the narrowed parts of the lateral extensions are approximately equal, while the height of the first panel is greater at the central region 15 than at the narrowed parts of the lateral extensions.

The headgear 10 has a rear portion 100 and a plurality of straps. Specifically, the headgear 10 has a pair of upper side straps 301 and 303, and a pair of lower side straps 302 and 304. The headgear also has a top strap 200.

In other forms, the headgear 10 may comprise a rear portion and two side straps, one for connecting to each side of the patient interface.

The straps may further be comprised of the same panel or panels as the rear portion.

One or more, or at least part of one or more of the straps may be formed from one or more fused panels according to the disclosure. Additionally or alternatively, the straps may be formed from another material such as a laminated foam. Where the straps are not formed from one or more fused panels they may still be joined to the remainder of the headgear by being fused to one or more of its panels.

While the upper side straps 301 and 303 are each shown in FIG. 11 as being formed by the same panel as a respective part of the top strap 200, each strap may be a separate part.

In other forms where there are two side straps on each side of the headgear adjacent straps 301 and 302, and 303 and 304, may be formed from one or more of the same panel or panels as each other. In still other forms each of the adjacent straps 301 and 302, and 303 and 304, and an adjacent part of the top strap 200 may be formed from one or more of the same panel or panels as each other.

While shown as including two strap parts, the top strap 200 may in some forms be a single strap which attaches back to the rear portion 100 of the headgear.

In some forms the top strap or straps 200 may be formed from one or more of the same panel or panels as the rear portion 100 of the headgear. In such forms the side straps 301-304 may also be formed from one or more of the same panels as the rear portion of the headgear, or one or more of the side straps 301-304 may be separate parts which are joined to the rear portion 100.

The straps of the headgear may include features 330 to facilitate the tensioning of the straps to the patient interface, or in the case of the top strap 200 to itself. Examples of such features 330 are shown in FIG. 10. These features 330 may for example be one half of a hook and loop fastener, where the other half may be provided by a surface of the headgear such as part of a strap or the rear portion.

While the headgear may be illustrated without any straps or other features for attaching to the patient interface it will be understood that a headgear or particularly a rear portion 100 of a headgear may include any suitable number or arrangement of straps or other fixtures as desired to facilitate its connection with and adjustment relative to a patient interface.

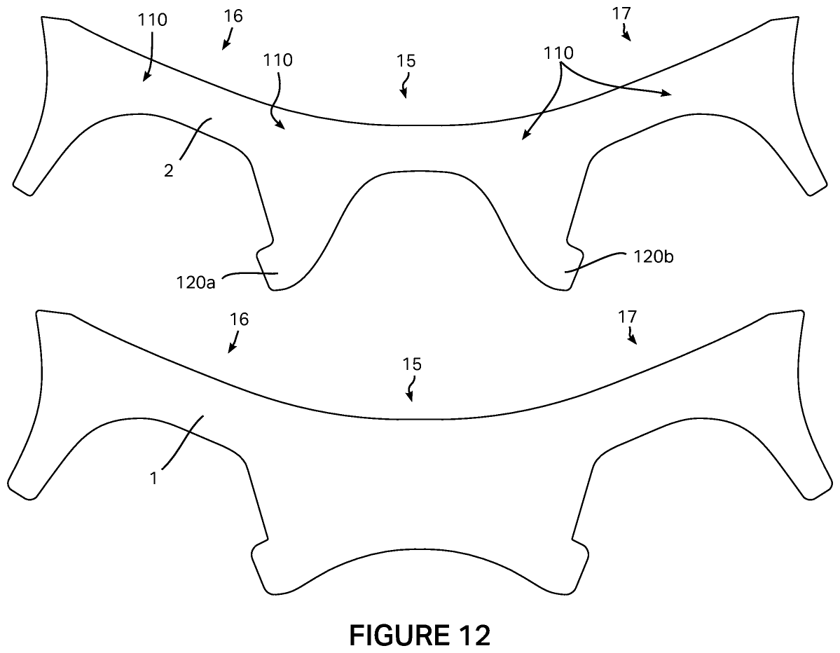

FIG. 12 shows a first panel 1 and second panel 2 which may form part of a headgear 10 according to an embodiment. The panels both have a central region 15 and two lateral sides 16 and 17. The central region 15 of the second panel has a cut-out at a lower part of it.

As seen in FIG. 12 the cut-out is located between a band 110 of the rear portion and two lower strap connection portions 120a and 120b of the headgear.

The first panel 1 and second panel 2 may be of the same material or of different materials. Particularly, the first panel 1 may be more stretchable than the second panel.

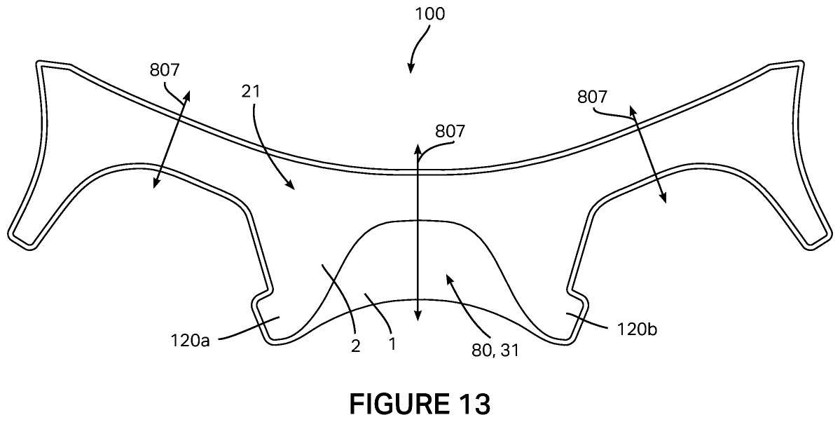

FIG. 13 shows the panels 1 and 2 of FIG. 12 overlaid on each other. The second panel 2 is located wholly within the first panel, so that the lapped region 21 defined by the panels has the same dimensions as the second panel.

The lapped panels define a first non-lapped region 31 about the external periphery of the second panel where there is a protruding border of the first panel 1, and a second non-lapped region 32 at the centre of the rear portion 200 defined by the cut-out of the second panel 2.

The panels may be sized so that at one or both of the lateral extensions 16 and 17 the height of the second panel may be about 70% to about 80% the height of the first panel at that same part.

As the headgear has only the thickness of the first panel 1 at the second non-lapped region 32 it may define a region which is thinner or more extensible than the remainder of the lapped region 21.

As the rear portion is to be located at the rear of a patient's head, the lower extent of the rear portion is to be located around an upper part of the patient's neck. This is a region which may have considerably variable geometry between people of different anatomies, and thus may be a point of discomfort for patients using a given headgear.

According to various forms where the first panel is of a stretch material the non-lapped region 31 may function to allow stretching to accommodate the geometry of a patient's neck. Accordingly, the non-lapped region 31 at the cut-out of the second panel 2 may be characterised as a stretch zone 80.

The stretching at the stretch zone 80 may be greater than a degree of stretching accommodated by at least adjacent portions of the headgear, and particularly than by the lapped region 21 as seen in FIG. 13.

While according to various embodiments the first panel 1 may fully lap the second panel 2, according to other embodiments the first panel 1 may only lap the second panel about a border of the cut-out of the second panel 2. By this configuration the headgear may still provide the previously described stretching function, but the first panel may be of a relatively smaller size.

The cut-out of the second panel relative to the first panel may be of a substantially semi-circular or crescent shape to define the stretch zone 80, as seen in FIG. 13.

The cut-out may be of increasing lateral size towards the bottom of the headgear, to allow for relatively greater expansion towards the bottom of the headgear to accommodate larger lower neck sizes.

The first non-lapped part of the first panel 1 about the second panel 2 may be a free edge, without a seam. It may be provided either with some edge treatment, or without any edge treatment.

In various forms, any edge treatment of the panel may be provided by the fusing of the panel or panels.

The absence of a seam and/or any edge treatment of the lower edge may increase the comfort of the headgear for a patient by providing continuous properties, such as stretch, stiffness, and thickness, across the whole of the first panel 1 lapped part with the second panel the whole way to the edges of the first panel.

As the first panel 1 beyond the second panel 2 is unsupported and may not be substantially load bearing, when in contact with the head of a patient it may roll or curl away from the patient's head. This may create a region of reducing pressure against the patient's head from the edges of the lapped region to the distal edge of the first panel 1.

Such a zone of gradually reducing pressure may have an edge softening effect, and provide for increased comfort for a patient, as opposed to a sharp drop in pressure across a hard edge which may make the patient more aware of the presence of the headgear or cause pressure irritation of the tissue at the edge.

The panels of the rear portion 100 may be fused together to join them and define the or part of the headgear.

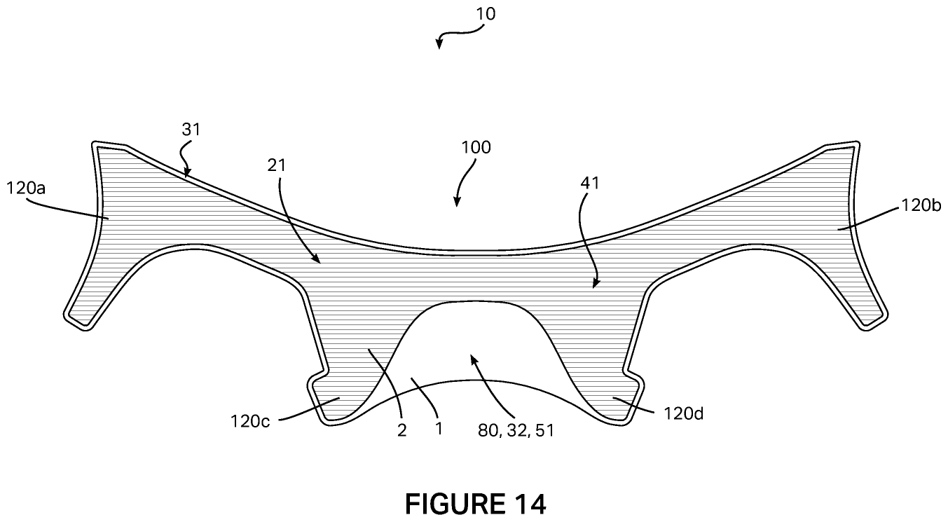

FIG. 14 shows the rear portion 100 of FIG. 13 where the panels have been fused together, such as by a weld applied to the panels. As illustrated by the shading in FIG. 14, the fused zone 41 covers the entirety of the lapped region 21, such that all the lapped parts of the first panel 1 and second panel 2 are fused together.

The first non-lapped zone 31 of the border of the first panel is not fused, and as such may retain a softer, more flexible, or more stretchable property than the fused panels at the fused zone 41.

By such a configuration the headgear may retain a desired degree of comfort about its edges.

While FIG. 14 shows the fused zone 41 extending across the whole of the lapped parts of the first panel and second panel, various embodiments of the headgear may utilise one or more non-fused zones.

Furthermore, while the headgear of FIG. 14 shows the fused zone 41 only covering the lapped panels, various embodiments may incorporate the fusing of all or parts of non-lapped regions. This may for example be to obtain the benefits of tolerance to misalignment of the panels or the fusing as described in relation to FIGS. 7A-8C.

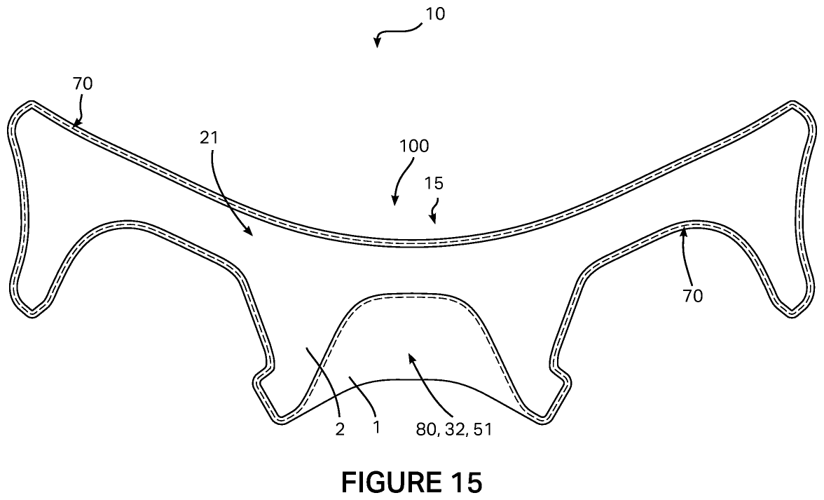

FIG. 15 shows the laid-up panels of FIG. 13, illustrating a boundary 70 of area to be fused which extends beyond the perimeter of the second panel 2.

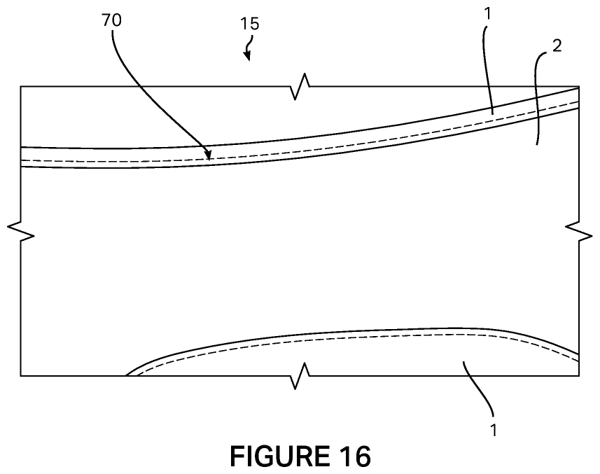

FIG. 16 shows a close-up view of an upper part of the central region 15, at the top of the cut-out of the second panel. The boundary 70 of the fusing extends to an external periphery of the second panel, being a margin or border beyond the peripheral extent of the second panel 2.

In FIG. 16 as illustrated by the boundary 70, fusing is to be applied both within the periphery of the second panel 2, and to the first panel 1 at an external periphery of the second panel 2, being the border between the perimeter of the second panel 2 and the line of the boundary 70. As illustrated in FIG. 16, fusing is to be applied to not only a periphery of the second panel 2, but to the entire area of the second panel.

While illustrated in FIG. 16 as being located at an external periphery of the second panel 2, the boundary 70 may be located anywhere between an external periphery of the second panel and within the periphery of the second panel. For example, boundary 70 may be located at the perimeter of the second panel 2. Where the boundary 70 is located within the periphery of the second panel 2, a border about the periphery of the second panel will remain unfused.

The distance a boundary 70 extends beyond the second panel 2 may be chosen according to an expected error in placement of the panels relative to the fusing equipment during manufacturing of the headgear.

The size of the boundary may be continuous about the external periphery of the second panel. In other configurations the size of the boundary may be variable around the external periphery of the second panel.

While in some embodiments the boundary 70 extending into the first panel 1 may result in a fusing of the first panel, in other embodiments the first panel 1 may be melted to a lesser degree than the second panel or even not at all.

For example, the first panel may be made of a material having a higher melting point than that of the second panel. Provided that the temperature of the materials when the fusing is provided do not exceed the melting point of the first material, the first material may not be fused when fusing is applied to it.

Where the fusing is applied by a high frequency welding the first panel may comprise less of a dipolar material than the second panel or may not comprise any dipolar material. Accordingly, the first panel may not be heated and melted by the high frequency welding which may be applied to it.

According to at least some embodiments the entire periphery of one or even all lapped regions of the headgear may be fused together. Edges of a lapped region where the lapped panels are not joined together may be unsightly. Un-joined edges of a lapped region may also introduce the potential for an undesired peeling apart of the lapped panels at an adjacent part where they are fused together.

Accordingly, in some embodiments of the headgear the entire periphery of each lapped region may be fused.

The rear portion 100 of a headgear may in some forms be configured to connect directly to a patient interface and may as such define the headgear. For example, the lateral ends of the rear portion 100 may be fitted with fixtures to attach to a patient interface or may otherwise be adapted to couple with the patient interface such as by an adhesive on one or both of the rear portion and the interface.

The rear portion 100 may be of a shape as illustrated in for example FIG. 13. The rear portion 100 may also have different shapes, such as which may include one or more straps formed from one or more of the panels which make up the rear portion 100.

In other embodiments one or more straps may be associated with the rear portion in order to provide the headgear 10. The straps may be associated with the rear portion 100 by any appropriate method, such as by fusing, gluing, or stitching.

Where the panels of the rear portion 100 are fused together it may be desirable to join the straps to the rear portion also by fusing. This may be conducted in discrete steps, where the straps are fused to the already fused rear portion. It may also be conducted in a single step, where the panels of the rear portion and the straps, whether panels or other materials such as a foam fabric-foam laminate, are all fused together in one operation.

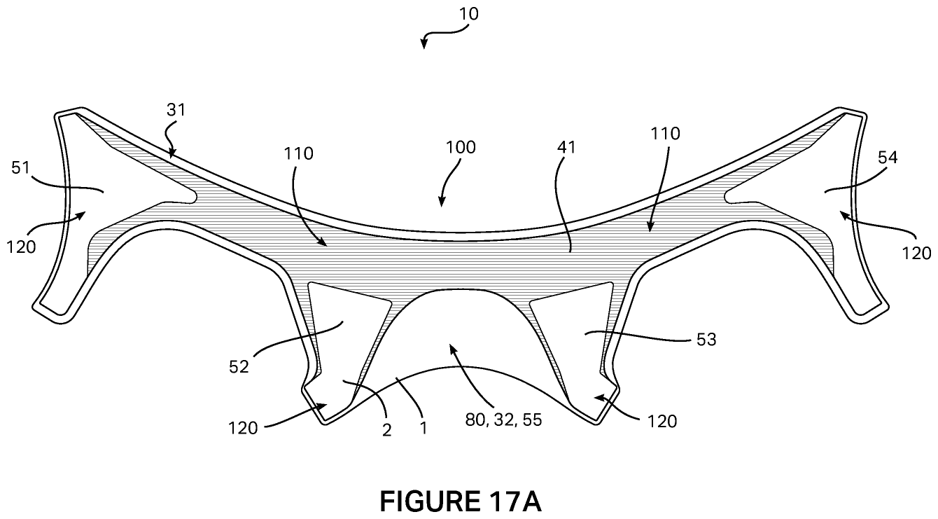

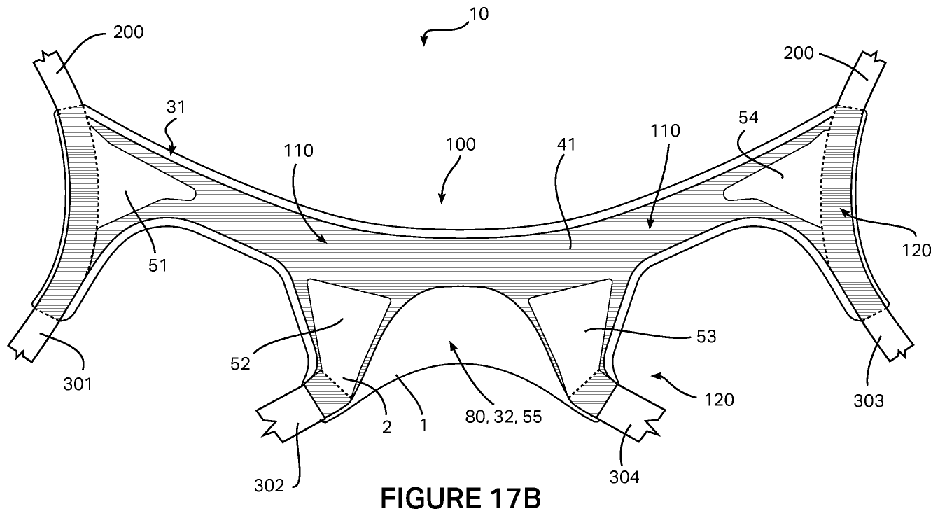

FIG. 17A shows another embodiment of a rear portion 100 of a headgear 10 where the lapped region includes a fused zone 41 and multiple non-fused zones 51-54.

According to some embodiments non-fused zones may generally be located at parts of the headgear which have relatively increased height. As such, the non-fused zones may be provided while still maintaining an amount of fused area across the lateral dimension of the headgear. FIG. 17A illustrates an example of this, where a contiguous fused band is provided along at least a significant part of the elongate direction of the headgear.

A headgear may define a band 110 and a plurality of strap connection portions 120. An example of a headgear having a band 110 is shown in FIG. 17A. The band 110 is a part of the headgear which extends around the patient's head and transfers load between the at least two connection points to the patient interface. The strap connection portions 120a-d depend from the band, and the straps either are wholly or partially defined by one or more of the same panels or are connected to them, such as by being fused together.

It will be appreciated that where straps are defined partially or wholly by one or more of the panels of the headgear at the strap connection portions 120 the shape of the first panel 1 and/or second panel 2 may be adapted from that shown for example in any one of FIGS. 13-17.

As seen in FIG. 17A, the headgear at the band 110 is largely a fused zone 41 and the strap connection portions are non-fused zones 51-54.

By this configuration the headgear may have a band of relatively reduced stretch, which may transfer loads between the sides of the headgear, and areas of relatively greater stretch at the strap connection portions. This may allow for stretch to accommodate the patient's physiology or different tensions on the straps.

FIG. 17B shows the rear portion 100 of FIG. 17A where it has been joined to a plurality of straps 301-304 and 200.

As seen in FIG. 17B, the straps have each been sandwiched between the first panel and second panel at each of the strap connection portions 120 and the first panel, respective strap, and the second panel have all been fused together at their overlapped parts.

As seen in FIG. 17B, the fusing of the headgear at the strap connection portions 120 will result in a fusing each of the first panel 1 and second panel 2 to respective sides of each respective strap.

Sandwiching of the straps between the first layer 1 and second layer 2 may provide the earlier described benefits of a double lapped joint and the consequent ability to reduce torsion at the joint under loading of the straps.

The headgear 10 as shown in FIG. 17B may be first formed as the rear portion 100 of FIG. 17A, then the straps joined as separate step. In other forms one or more of the straps may be joined to the respective panels in the same step as part or all of those panels are fused to form the rear portion 100 of FIG. 17A.

As seen in FIG. 17B, when the straps are fused to the rear portion they cause there to be a continuous perimeter of the second panel 2 which is fused. This may prevent loose edges of the second panel such as may allow for peeling of the first panel and second panel apart.

It will be appreciated that any embodiment of a rear portion 100 described herein having a non-fused zone of the lapped panels at a strap connection portion may be wholly or partially fused there when the straps are joined.

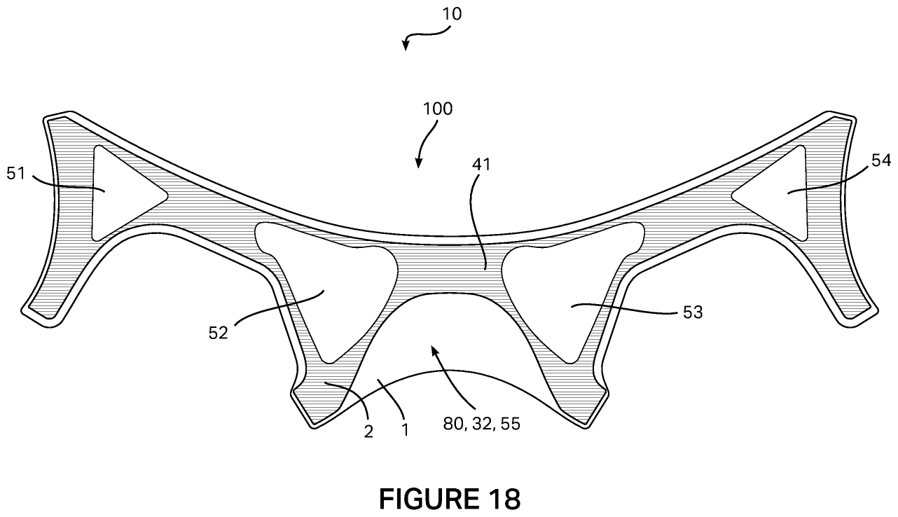

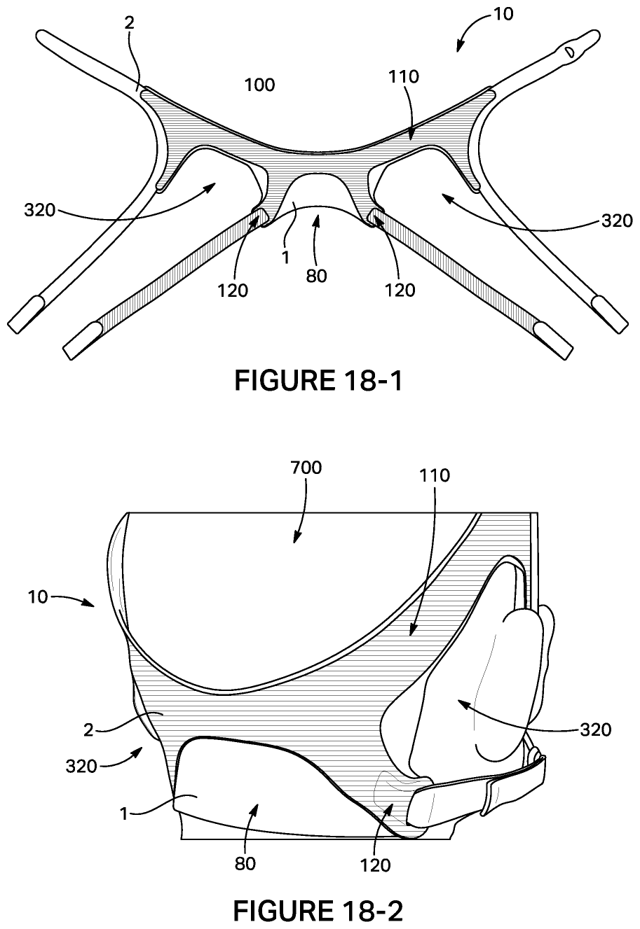

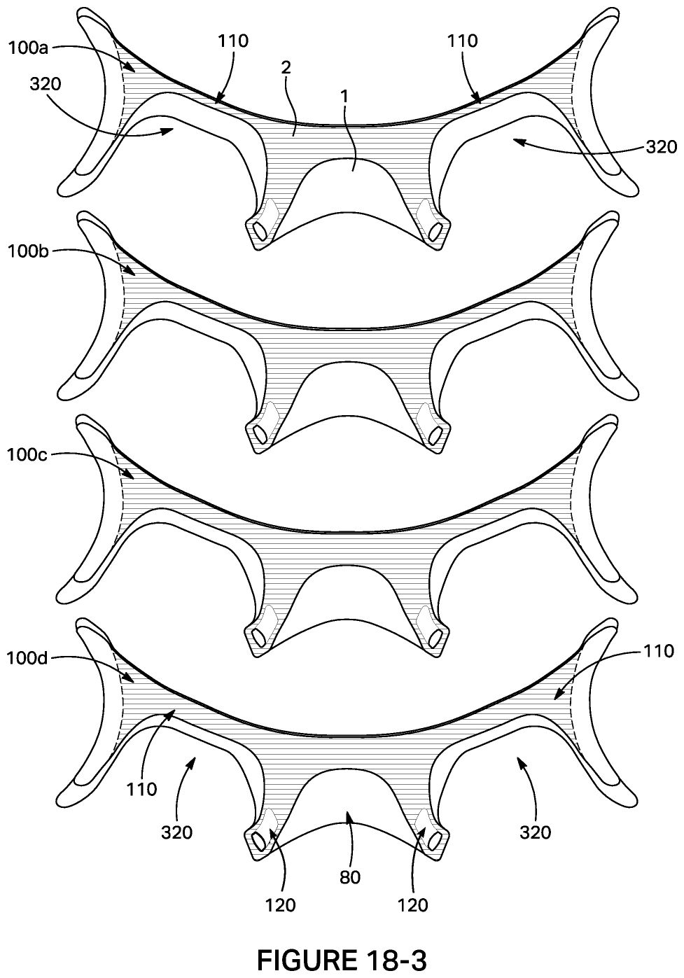

Another embodiment of a rear portion 100 is shown in FIG. 18, where the non-fused zones 51-54 are all enclosed within an uninterrupted fused zone 41 of the lapped panels 1 and 2.

The fused zone or zones may be discrete or two or more fused zones may define an uninterrupted fused area, as is seen in FIG. 18.

The enclosing fused parts of the panels may affect how loads are transferred through the headgear, as loads may be transferred from the straps to the fused strap connection portions, and then through the fused portions of the band of the headgear.

This may provide for a rear portion of a headgear having relatively less overall extensibility than that of the embodiment of FIGS. 17A and 17B.

The non-fused zones within the fused zone 41 of FIG. 18 may be of relatively greater stretch than the fused zone 41. The placement of the non-fused zones 51-54 at the widest parts of the lapped region may reduce the overall resistance of the lapped regions at those parts to stretch.

Apart from at the stretch zone 80, the size of a non-lapped part of the first panel 1 may be the same or substantially the same about the remainder of the non-lapped part of the first panel. Alternatively, non-lapped parts of the first panel other than the stretch zone 80 may vary in size. For example, the non-lapped amount of the first panel 1 at an ear loop 320 may be greater or less than a non-lapped amount of the first panel 1 at an opposite side of the headgear around a top of the band part 110 of the headgear.

The size of the non-lapped part of a first panel 1 about the second panel 2 may be referred to as a border of the first panel. As seen in the configuration of FIG. 18, the border formed by the non-lapped parts first panel 1 about the ear loops 320 and along the upper edge of the rear portion 100 is of a substantially continuous width.

While the border in FIG. 18 about the ear loops 320 and the upper edge of the rear portion 100 are of substantially the same size, in other configurations the border may be different widths in different regions. Customising the width of the border of non-lapped panel allow for providing local variation in the edge softness of the headgear when worn. Where the first panel is a stretch material, controlling the width of the border of non-lapped panel at different regions may allow deformation along the border to be controlled and recovered, such may occur at the stretch zone 80.

For example, in some configurations the first panel 1 may have a locally increased width of the non-lapped border at the or part of the ear loops 320. This may function to prevent or limit the headgear from lifting away from the patient's head when worn.

FIGS. 18-1 and 18-2 are, respectively, a lay-flat view of another embodiment of a headgear 10 and a partial rear view of the headgear 10 when worn. As seen in FIG. 18-1 the non-lapped portion of the first panel 1 at the sides of the ear loop adjacent the stretch zone 80 are of a locally increased width.

Between the respective strap connection portions 120 and the band 110 the non-lapped portion of the first panel 1 has a greater radius of curvature, such that the non-lapped portion between the connection portions 120 and the band 110 defines a crescent shape.

When worn by a patient, such as illustrated in FIG. 18-2, the forces acting on the headgear 10 may in at least some circumstances urge the headgear to lift away from the patient's head at or towards the base of the curve between the strap connection portions 120 and the band 110, behind the patient's ears. If the headgear experiences these forces, the locally widened portions of the non-lapped first panel may aid in limiting or preventing the headgear from lifting away from the patient's head between the strap connection portions 120 and the band 110.

FIG. 18-3 shows four different configurations 100a-d of a rear portion 100 of a headgear 10. Each of the rear portions 100a-d have a first panel 1 which is partially lapped by a second panel 2, with a non-lapped portion of each first panel 1 forming a border about various parts of the periphery of the rear portion. In particular, the non-lapped portions define a border at the rear stretch zones 80 and a border 83 at the ear loops 320.

Each of the rear portions 100a-d illustrate different configurations of the width of the non-lapped border of the first panel 1 at the ear loops 320. Such different widths of the border may be provided by increasing the local size of the first panel 1, or as in the configuration of FIG. 18-3 by decreasing the local width of the lapping second panel 2. In each progressive one of the rear portions 100a-d the second panel 2 at the ear loops 320 is of a relatively decreased width. In the case of the rear portion 100a the border 83 is approximately half the width of the adjacent part of the second panel 2. For the rear portion 100b the border 83 is approximately the same width as the adjacent part of the second panel 2. For the rear portion 100c the border 83 is approximately double the width of the adjacent part of the second panel 2. And finally for the rear portion 100d the border 83 is approximately three times the width of the adjacent part of the second panel 2.

Where the first panel 1 is a stretch panel and the second panel 2 is a non-stretch panel, decreasing the width of the second panel 2 at the ear loops 320 may increase the ability of the headgear to conform in this region to the shape of the patient's head.

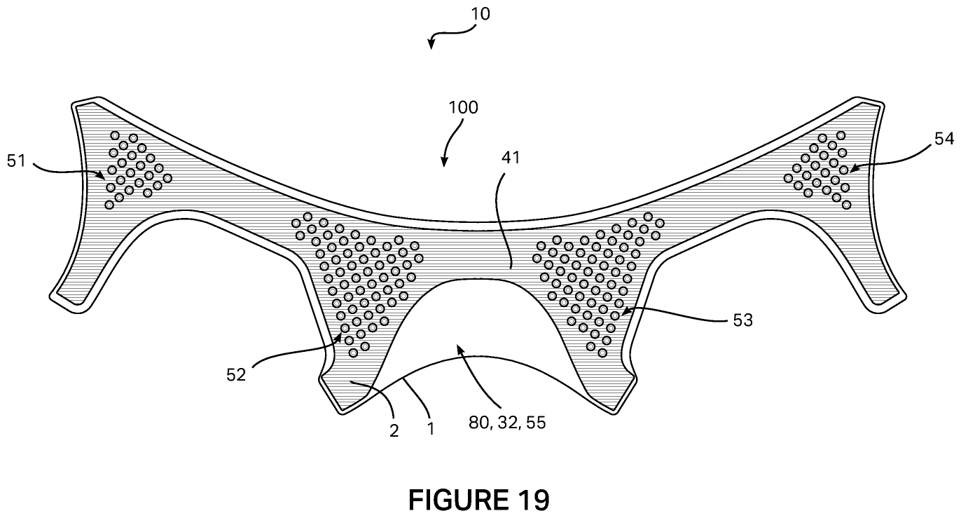

Another embodiment of a headgear is shown in FIG. 19. In FIG. 19 a substantial entirety of the lapped part of the headgear is fused to define a fused zone 41. As previously described, it will be understood that the fused zone 41 may extend beyond the second panel 2 and may encompass part or all of the non-lapped region of the first panel 1.

The headgear of FIG. 19 has non-fused zones 51-54 which are provided as a cluster of multiple small zones. As seen in FIG. 19 each small zone is circular; this shape may minimise tight geometries and accordingly reduce the risk of arcing or burning of the panels around the boundary of each small non-fused zone and the fused zone.

Where the fusing results in a thinning or flattening of the fused panel or panels, the configuration of FIG. 19 will provide a headgear surface with clusters of raised dots or bumps at each of the groups of non-fused zones 51-54.

This may provide a tactile feature for a user, such as helping the user orient themselves to the headgear by the feel of the raised bumps.

The cluster of small adjacent non-fused zones may also act together to create a stretch zone at which the headgear may have relatively increased extensibility.

The raised bumps at the non-fused zones may be more prominent at one or the other of the internal and external surfaces of the headgear by the selection of different panel materials. For example, relatively more prominent bumps may be formed at the external surface of the headgear than at the internal surface of the headgear where the panel at the external surface is compressed more by fusing than the panel at the internal surface.

It may be desirable to manufacture a single size of a headgear, or at least a minimal number of different sizes. It may also be desirable for a given headgear size to accommodate a broad range of patients.

A given size of a headgear may have certain dimensions and proportions of the rear portion 100, and also certain strap lengths.

To increase the ability for a given headgear size to accommodate different patient geometries, the straps may be lengthened. This may allow the headgear to fit patients having larger geometries. However, straps long enough to accommodate larger patients may cause issues for users of the same size headgear who have smaller geometries.

For example, where the strap ends are secured by fixtures on the straps or of the straps, such as by a hook or loop part of the strap end connecting to the other of a hook or loop part of the strap, for patients with smaller geometries the strap ends may return back past the base of the strap. This may make a given size of headgear unsuitable for patients with smaller geometries.

Where a panel is of an unbroken loop material and the fusing process is such that it reduces a density of unbroken loops at the surface of the unbroken loop material, one or more non-fused zones may be utilised to maintain an area of unbroken loops. This may for example allow the fixing of a strap fixture to the headgear at the one or more non-fused zones.

For example, as seen in FIG. 19 the cluster of small non-fused parts of the headgear at each of the non-fused zones 51-54 may provide sufficient density of unbroken loops to allow the fixing of a strap fixture having a hook part of a hook and loop fastener to the headgear at one or each of the non-fused zones 51-54.

The location of one or more non-fused zones may be such as to provide an extension of the region to which a strap fixture may be fixed.

For example, as seen in FIG. 17B the first non-fused zone 51 and fourth non-fused zone 54 are each located along a line of extension from parts of their respective straps 301 and 200 and 303 and 200. Similarly, at least part of the second non-fused zone 52 and third non-fused zone 53 is located at a line of extension from their respective straps 302 and 304.

While a non-fused zone may be positioned along a line of extension from a strap at a respective strap connection portion 120 or along a line of extension from any other distal part of the strap, it will be appreciated that a non-fused zone may be located anywhere on a headgear that corresponds to where it is desired for a user to be able to attach a strap fixture.

The rear portion 100 of FIG. 18 shows a further example of non-fused portions 51-54 which may allow for attachment of a strap fixture. As seen in FIG. 18 the middle non-fused zones 52 and 53 encompass a significant portion of the lapped parts of the panels adjacent the strap connection portions.

Also as illustrated in FIG. 18 the fused zones 51-54 are shaped so they increase along a notional line of extension from where straps would be connected to their strap connection portions, such as is illustrated with the straps of FIG. 17B.

Non-fused zones to which a strap can be affixed may be located directly adjacent to the base of a strap.

In forms where there is a continuous perimeter of the lapped panels which are fused together, the non-fused zone or zones may be located within the fused perimeter but as close as practicable to the base of a strap. This may prevent or minimise strap lengths at which the strap fixture cannot be fixed to the non-fused zone.

In other forms there may be a fused part of the panels which is located between the base of a strap and a non-fused zone. This is exemplified by the part of the fused zone 41 between the strap 302 and then non-fused zone 52 of FIG. 17B.

Where there is a fused zone between a base of a strap and a non-fused zone of the headgear to which the strap end can connect, the fixture of the strap end may be sized so as to be able to span the gap between strap base and the non-fused zone. For example, in the configuration of FIG. 17B a strap fixture of the strap 302 may be of greater size along the length of the strap than the dimension of the fused zone 41 between the base of the strap 302 and the non-fused zone 52.

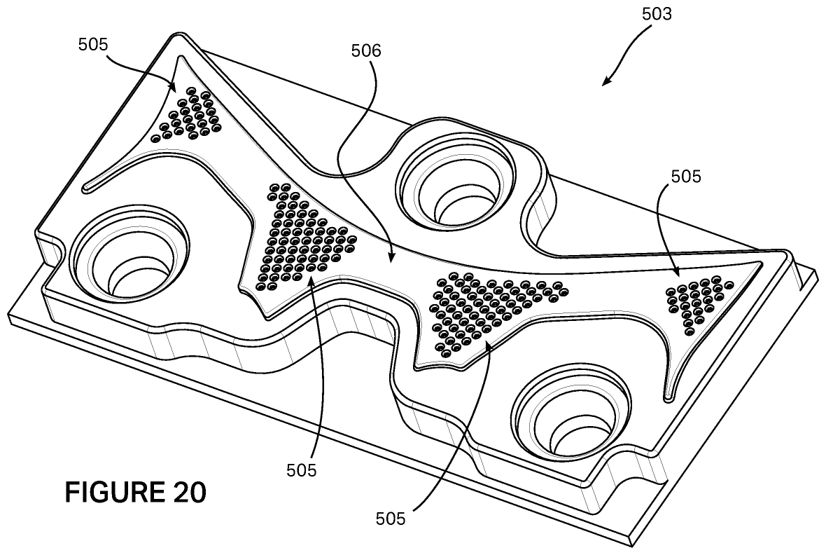

FIG. 20 illustrates a configuration of a die of a welding apparatus 500, for example either an upper die 503 or lower die 504 for use in fusing a headgear as shown in FIG. 19.

As seen in FIG. 20 the die 503 has a die face 506 which corresponds to the size and shape of the desired fused zone 41 of the headgear. A plurality of small circular regions 505 are recessed from the die face 506. At these recessed parts the die may press less strongly or not press against the adjacent panel of the headgear during fusing. This may result one or both of a reduced compression of the panel adjacent the die at the recessed parts and a decreased degree of or no welding of the panel or panels at the recessed parts.

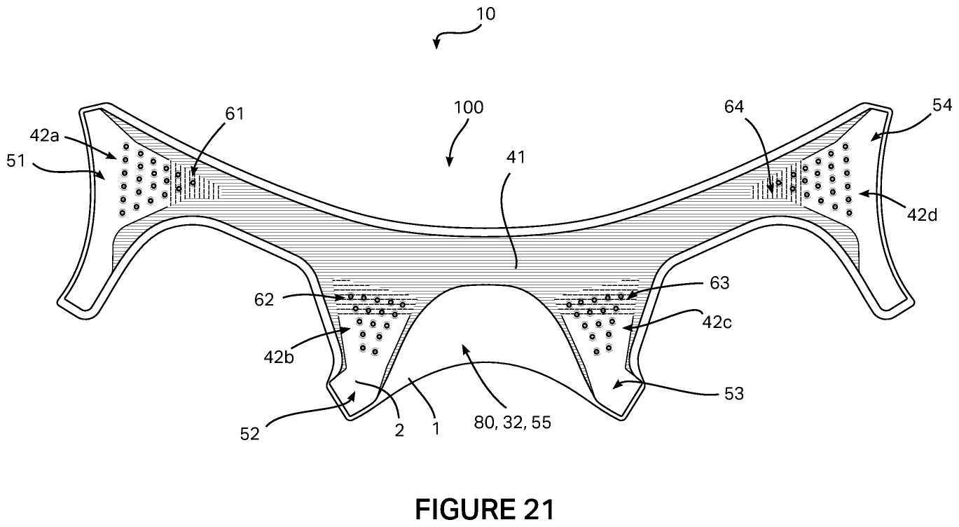

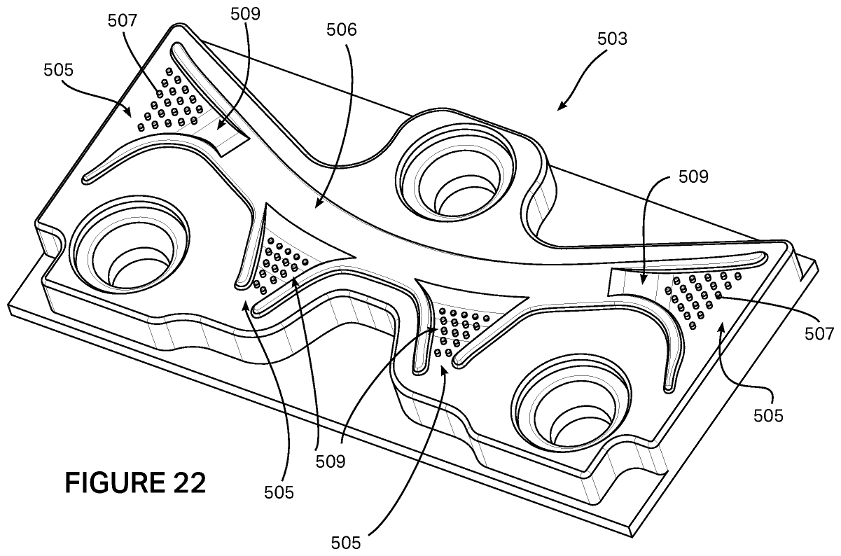

FIG. 21 illustrates a further embodiment of a fused headgear, and FIG. 22 shows an example of a die 503 which may be used to manufacture the fused headgear of FIG. 21.

As previously described, the headgear may have a transition zone of a fused panel or panels. In a transition zone the degree of fusion of the panel or panels may transition between that of a non-fused zone and that of a fused zone.

A transition zone may be located between a non-fused zone and a fused zone.

In other configurations transition zones may be provided within a non-fused zone or within a fused zone, or between two non-fused zones or between two fused zones. In such cases the transition zones may define an area of partial fusing of the panel or panels.

FIG. 21 illustrates a fused headgear 10 having a fused zone 41 across a band part of the headgear, and four non-fused zones 51-54 at strap connection portions of the headgear.

The headgear also has a set of second fused zones 42a-d which are provided as a cluster of small individual zones. These are the inverse of the non-fused zones of the headgear of FIG. 19, and instead define spots of material which are fused.

These may for example be formed by a spot welding of the headgear.

Where the panels are compressed or thinned when they are fused, the second fused zones 42 define spot indents or recesses in the headgear.

As seen in FIG. 21 the lateral second non-fused zones 42a and 42d are of a triangular overall shape, which tapers inwardly towards the central portion of the headgear. The base of the triangular shape is located adjacent to the respective strap connection portions 51 and 54, while the tip of the triangle is located away from the respective strap connection portions 51 and 54.

As seen in FIG. 21 the narrowing part of the second non-fused zones 42a and 52b are located within the transition zones 61 and 64 respectively.

The second non-fused zones 42b and 42c are also of a substantially triangular shape. However, unlike the second non-fused zones 42a and 42d, the second non-fused zones 42b and 42c are oriented so that their narrowed part is located towards their associated strap connection portions 52 and 53, and the opposite base part is located towards the band of the headgear.

As seen in FIG. 21 the wider parts of the second non-fused zones 42b and 42c are located within the transition zones 62 and 63, rather than the narrowed part of the second non-fused zones 42a and 42d in relation to their respective transition zones 61 and 64.

As seen in FIG. 21 the second non-fused zones 42a and 42d are of greater size adjacent to their respective strap connection portions 51 and 54 than the second non-fused zones 42b and 42c adjacent to their respective strap connection portions 52 and 53.

The size of a fused zone comprised of a cluster of smaller fused parts or spots may be either greater or less than those shown in FIG. 21. Similarly, the size of the smaller fused parts and their proximity to one another may be either greater or less than what is illustrated in FIG. 21.

Where a cluster of smaller fused parts make up a fused zone the fused parts may be uniform or non-uniform in size and relative location to each other.

Each smaller fused part may be fused to the same degree, such as is seen in the illustration of FIG. 21. Alternatively, one or more of the smaller fused parts may be fused to a relatively greater or lesser degree. As with a transition zone, the degree of fusing of the smaller fused parts may vary across a fused zone which is made up of a cluster of smaller fused parts. For example, a degree of fusion may decrease towards one or more sides of the fused zone, or may decrease towards the entire periphery or perimeter of the fused zone.

The size and shape of the smaller fused parts and their density and arrangement within a fused zone 42 may be such as to provide a desired pattern or texture to the headgear or part or parts of the headgear.

The configuration of fused and non-fused zones, particularly where patterns of fused nor non-fused zones are used, may be utilised to customise and particularly to localise properties of the headgear such as breathability, its ability to wick moisture, its edge properties, hand feel, drape, and its stretch and recovery.

For example, particularly where a fusing of the headgear compresses or thins the headgear at the fused part, clusters of fused spots may be used to give the headgear a waffle texture. Such a waffle texture may include a pattern of depressed spots. Similarly, clusters of non-fused spots may be used to give the headgear an inverse waffle texture.

The headgear of FIG. 21 further has four transition zones 61-64. These are each located between an associated one of the non-fused zones 51-56 and a part of the fused zone 41.

At each of the transition regions 61-64, as illustrated by the lesser density of shading, the first and second panels 1 and 2 may be fused together to a degree between that of the fused zone 41 and that of any of the non-fused zones 51-54.

A transition zone may define a single degree of fusion, for example where the panels are fused together to have approximately half the peel strength of a fused zone.

The shapes of transition zones 61-64 may vary. As shown in FIG. 21, transition zones 61 and 64 vary in size from being narrow narrower at a more central part of the headgear to a wider configuration the non-fused end. Conversely, as shown in FIG. 21 the transition zones 62 and 63 have a widest dimension at a part that is most central on the headgear.

Alternatively, a transition zone may define a graduation in the degree of fusion. For example, as seen in FIG. 21 the transition zone 62 may have a first relatively lesser degree of fusion adjacent to the non-fused zone 52 and a second relatively greater degree of fusion adjacent to the fused zone 41.

Where the degree of fusion varies across a transition zone it may do so in steps or by a gradient. The gradient may be continuous, or it may vary across the transition zone.

Through the use of transition zones, particularly where the degree of fusion varies across the transition zone, a smoothing effect may be provided between the properties of adjacent fused zones and non-fused zones.

For example, where one or more of the thickness, extensibility, recoverability, softness, or surface texture of a panel or panels are changed by fusing, a transition zone may graduate the transition between these different properties. This may provide for one or more of improved performance, increased comfort, or improved feel of the headgear.

The comfort and also performance of the headgear may particularly be affected by the use of transition zones. Changes between different regions of the headgear, for example a relatively less extensible and load-carrying part and an adjacent relatively more extensible part of the headgear may induce pressure concentrations at the boundary when the headgear is worn by a patient. Even where pressure concentrations are not induced, the sharp transition between regions of different properties may create a pressure sensation for a patient along the boundary.

As such, transition zones may allow for graduation in the change of properties of the headgear between two different regions, and accordingly may provide for increased comfort for a patient.

Loads may be applied in use from the strap connection portions at the non-fused zones 51-54 and loads may be transferred to the band of the headgear at the fused zone 41. Accordingly, the location of the transition zones 61-64 of the headgear of FIG. 21 are in between the non-fused and fused zones and may as such provide for a graduation in at least extensibility of the headgear along the load transfer paths.

Various versions of the headgear 10 of FIG. 21 may be provided with or without one or more of the sets of second fused zones 42.

In the embodiment of FIG. 21 the set of second fused zones 42 may act to pin the panels together at that part of the associated non-fused zone 51-54 and transition zone 61-64. This may prevent an undesired separation of the first and second panels.

The set of second fused zones 42 may additionally or alternatively also act as a transition element, effectively increasing a density of fused area or an average degree of fusion within the associated non-fused zone 51-54 and transition zone 61-64.

The headgear of FIG. 21 may have one or more straps attached to it. For example, side straps 301-304 and potentially also top straps 200 may be attached to the headgear 10 at one or more of the strap connection portions 120.

The one or more straps may be attached to the panels of the rear portion of the headgear by any desired method, such as by fusing, by an adhesive, or by use of a fixture.

According to some embodiments, the straps may be fused to one or both of the first panel 1 and second panel 2.

In particular, as seen in the configuration of FIG. 21 one or more straps such as are illustrated in FIG. 11 may be sandwiched between the free ends of the first panel 1 and second panel 2 at the non-lapped regions 51-54, and the panels and strap fused together.

While the rear portion of the headgear is illustrated in FIG. 21 as having been fused prior to the attachment of any straps, it will be appreciated that the step of fusing straps with the rear portion may be integrated with the step of fusing the panels of the rear portion.

A weld die 503 used to fuse the rear portion of the headgear similar to that of FIG. 21 is shown in FIG. 22.

As seen in FIG. 22 the die has a die face 506 which corresponds to the fused zone 41. It also has four recessed regions 505 which correspond to the four non-fused zones 51-54. The transition zones 61-64 of the headgear are defined by the sloping transition regions 509 of the die, which graduate from the level of the recessed regions 505 to the level of the die face 506.

The set of second fused zones 42 are defined by the plurality of posts 507 which are at the level of the die face 506 and are defined by the recessed parts of the die.

The posts 507 may rise up to the same level as the die face 506.

The posts 507 may extend to some level between that of the recessed level 505 and the die face 506.

The posts 507 may be all of the same height. Alternatively, one or more of the posts may be of different heights.

In particular, the height of the posts may vary from a side of each second fused zone which is adjacent a non-fused zone to a side of each second fused zone which is adjacent a fused zone. For example, the height of the posts may increase towards a fused zone, so as to an increasing degree of fusion at the posts towards the fused zone.

The transition zones of a headgear may have a gradient of their degree of fusion in one direction, such as is illustrated in the transition zones 61-64 of the headgear FIG. 21. Here the degree of fusion increases towards away from the adjacent non-fused zone and towards the fused zone at the band of the headgear.

It may however be desirable to provide for gradients of the degree of fusion from all adjacent parts of either or both of a non-fused zone or a fused zone.

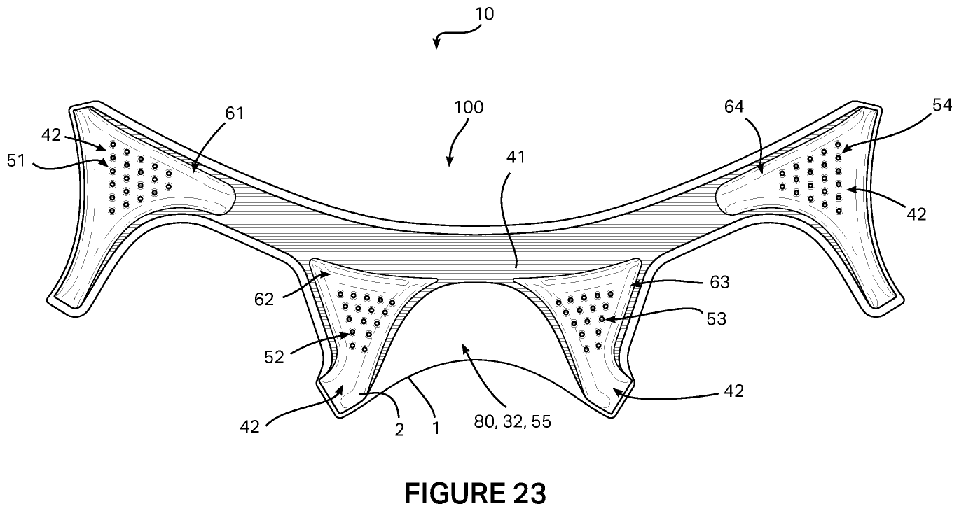

FIG. 23 shows an example of a headgear 10 of a similar configuration to that of FIG. 21, but where the transition zones 61-64 have a gradient of fusion about their entire perimeter.

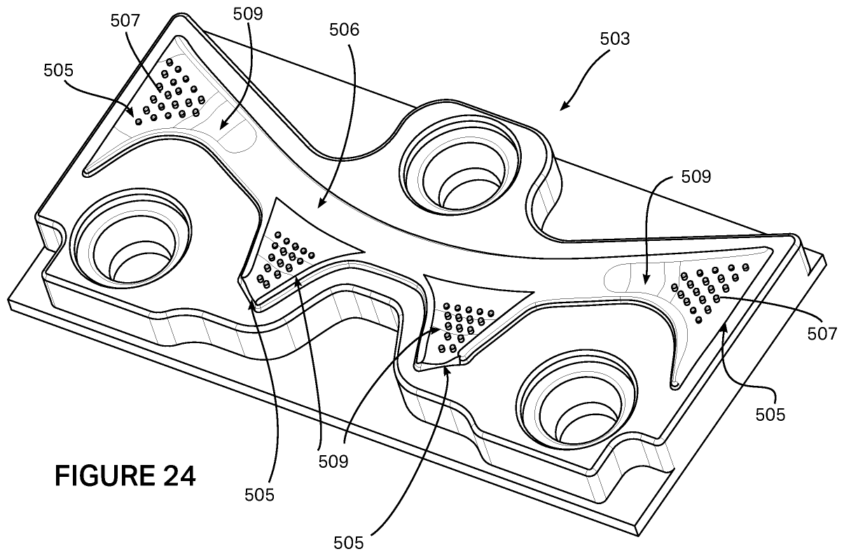

A die for making a headgear of the configuration of FIG. 23 is shown in FIG. 24. In FIG. 24 the transition regions 509 each define smooth curves of die height between the recessed regions 505 and the die face 506.

A headgear made using such a die may have increased comfort, as all transitions between welded and non-welded parts of the headgear with their different properties may be gradual rather than abrupt.

A headgear may include one or more user identifiable indicia, such as a brand, a model name or number, sizing information, a serial number, regulatory information, or other such features. Such indicia may conventionally be additively applied to the headgear, for example by stitching or adhesion of a label.

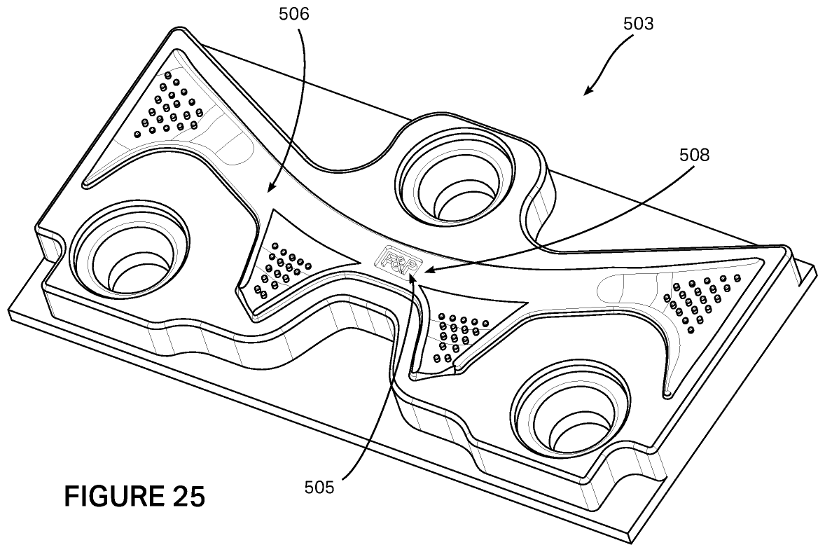

FIG. 25 shows another die for use in manufacturing a headgear. The die 503 of FIG. 25 additionally comprises a set of indicia features 508. The indicia features 508 are a combination of regions at the die face 506, at a recessed height, and potentially at various transition heights therebetween. By the combination of such features one or more indicia may be formed into the headgear when the panels are fused. The indicia may be identifiable from the resulting combination of fused, non-fused, and potentially transition zones. For example, where the panels are made thinner when they are fused, the indicia may be identifiable in the combination of relatively raised and lowered parts of the headgear.

Where the colour or surface texture of the panels are changed by their being fused, the indicia may additionally or alternatively be identifiable through differences in these features.

It will be appreciated that the indicia features 508 of the die will define in relief the resulting features of the indicia on the headgear; recessed parts of the die will define relatively raised parts of the headgear, and vice-versa.

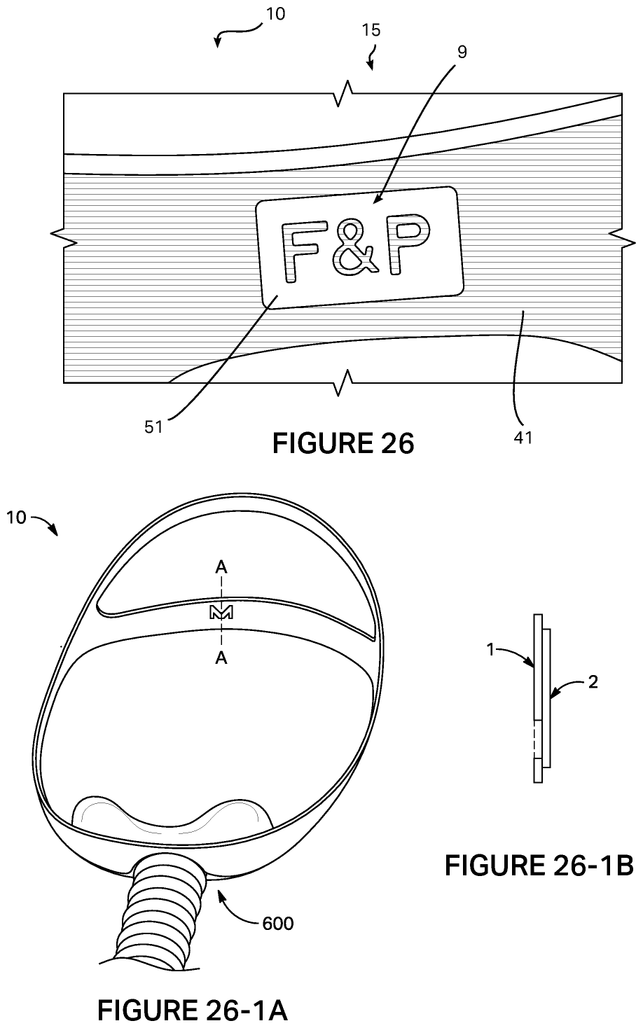

FIG. 26 shows a partial view of a fused headgear 10 having an indicium 9 formed in it by fusing with a die having indicia features 508 such as those of the die of FIG. 25.

As seen in FIG. 26, the indicia 9 is defined by a non-fused zone 51 and separate fused zones 41-43 which form the shape of the component letters or symbols of the indicia, in this case a brand.

In other configurations the indicia 9 may be formed without a surrounding differentiating part as with the non-fused zone 51 in FIG. 26. In such a case the component letters or symbols of the indicia would be defined by a non-fused zone, so as to distinguish the surrounding fused parts of the headgear.

The application of an indicia by fusing of the panels may be provided as a separate step to the fusing of the panels to join them together.

In various embodiments the forming of the indicia may be done in the same step as the fusing of the panels to join them together. This may allow for a simplification of the manufacturing process, as the indicia does not need to be provided as a separate manufacturing step.

While illustrated in FIG. 26 as being at a middle of the central region 15 of the headgear 10, and within a fused zone, it will be appreciated that indicia 9 may be provided at any desired location of the headgear. Similarly, the headgear may be fused so as to present the indicia at one or both of the internal or external surfaces of the headgear.

In addition to or alternatively to forming indicia by selective fusing of a panel or panels, indicia may be formed by selectively removing material of a panel or panels.

For example, FIG. 26-1A shows a headgear 10 which includes an indicia 9. FIG. 26-1B shows a cross-section through the line A-A of FIG. 26-1A. As seen in FIG. 26-1B, the headgear at the cross-section has a first panel 1 which is partially lapped by a second panel 2. The first panel 1 however has had material removed from it to form the shape of the indicia 9. The first panel 1 and second panel 2 have been fused together to define the indicia 9 in relief through the removed material.

Such a configuration may provide a clearly visible indicia particularly where the first panel 1 and second panel 2 are of different colours or surface textures.

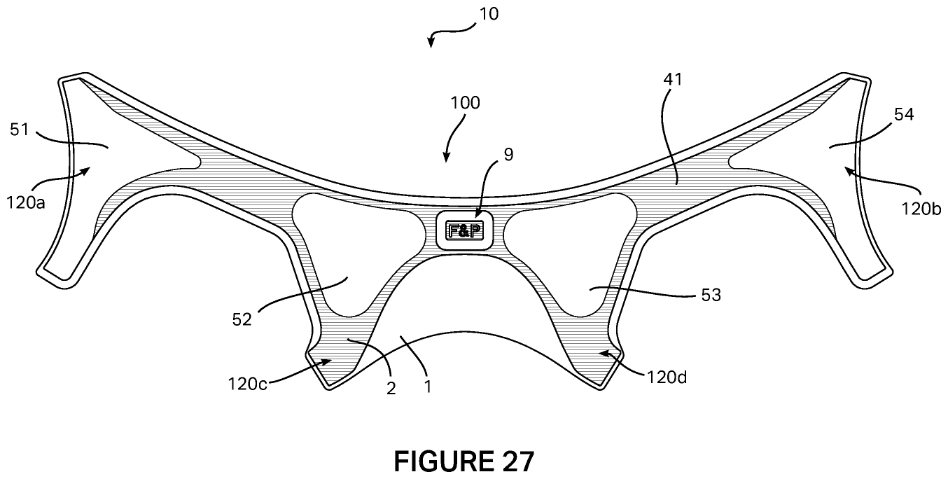

FIG. 27 shows another embodiment of a headgear 10 which includes a fused indicium 9. The headgear 10 has non-fused zones 51 and 52 at the strap connection portions 120a and 120b at the lateral ends of the headgear and is fused at the two more central strap connection portions 120c and 120d.

There are two large non-fused zones 53 and 54 above the strap connection portions 120c and 120d.

The indicia 9 is located centrally at the lapped part of the first panel 1 and second panel 2 above the cut-away of the second panel 2.

One or more straps may be attached to the headgear of FIG. 27 as previously described. While the first panel and second panel are fused together at the lower strap connection portions 120c and 120d the fused panels may be lapped against a strap and fused together with it.

The panels to form the headgear may need to be placed in the welding apparatus in particular positions relative to each other and to the dies of the welding apparatus. This may be necessary to provide the desired configuration of lapped and non-lapped regions of the headgear, and to provide the desired location of fused and non-fused zones.

Accordingly, it may be desirable to include one or more locating features either on or of one or more of the panels, to align them either or both with each other and with one or both of the dies.

FIG. 28A shows an embodiment of a layup for a headgear before it is fused. The layup includes a first panel and fully lapped second panel 2. The first panel 1 includes a number of alignment features 8 which are provided on a sacrificial part 7 of the first layer.

The alignment features, holes in the embodiment of FIG. 28A, may mate with corresponding features of the die or dies of the welding apparatus. This may allow the first panel to be placed in a desired location relative to the dies.

One or more of the panels in a layup may include such alignment features. For example, both the first panel 1 and second panel 2 may include alignment features. The alignment features may be discrete or may overlap with each other such when the panels are lapped in their intended positions. For example, both the first panel and second panel may have alignment features that are co-extant when the panels are located relative each other in their desired positions. By this configuration the panels may be located relative to the dies, and the panels may be located relative to each other.

In some forms the sacrificial parts 7 may themselves act as the alignment features, for example by aligning with some part of the die or dies.

The sacrificial parts 7 are to be separated from the panel or panels before the manufacture of the headgear is completed. The separation of the sacrificial parts may be by, for example, a stamping or cutting of the sacrificial parts away from the remainder of the panel or panels.

In some forms the sacrificial parts may be stamped to separate them from the panels, and the step of stamping may be integrated with the step of fusing the panels together. To this end the dies may include a cutting surface which may act on the panels when the dies are brought together to fuse the panels.

In other forms the sacrificial parts may be separated by a melting or burning of the panels during the fusion of the panels, for example in the case of high frequency welding by allowing an arcing between the dies where the sacrificial parts are to be separated from the remainder of the panels.

In some embodiments the alignment features 9 may not be provided on a sacrificial part which is removed from the layup, and instead are provided on or within the rear portion of the headgear itself. For example, one or both of the panels may include one or more holes for alignment of the panels, and those holes remain a feature of the headgear when it has been fused.

FIG. 28B illustrates the headgear 10 of FIG. 28A after it has been fused and stamped. The headgear includes a main fused zone 41, sets of spot welds 42, and four non-fused zones 51-54.

While stamping or cutting may be employed to separate sacrificial parts from the headgear to facilitate alignment of one or more panels as previously described, other parts of the headgear may additionally or alternatively be stamped or cut.

Accordingly, the headgear may have material removed from it by cutting or stamping. This may include either or both of removal of peripheral parts of one or more panels, and the removal of internal parts of one or more panels.

For example, parts of a lapped region and particularly of a fused zone of a lapped region may be removed from the headgear. The removal of these parts may for example reduce the weight of the headgear. The removal of parts may also improve the breathability of the headgear.

Cutting or stamping of one or more of the panels of the headgear may also be configured to create one or more indicia such as a brand, a model name or number, sizing information, a serial number, regulatory information, or other such features.

Cutting or stamping of one or more of the panels of the headgear may create an indicia by making one or more holes through the headgear in the shape of the indicia.

An indicia may additionally or alternatively be formed by removing part of one layer at a lapped region. The indicia may then be identifiable through the surface features of the headgear where the material has been removed. The indicia may be identifiable through colour or texture where the lapped layers are of different colours or textures.

Where the indicia is cut or stamped into one panel at a lapped region, it may be possible to provide an indicia which is identifiable or at least primarily identifiable from only one side of the headgear. For example, where a panel to be most proximate to the user at the inside surface of the headgear is stamped while an adjacent panel or panels towards or at the external surface are not stamped, the indicia may only be displayed at the inside surface of the headgear.

Any removed parts of the headgear may be removed prior to the fusing of the headgear.

Parts of the headgear may be removed in the same step as the fusing of the headgear, such as by a die-cutting of the headgear which occurs when the fusing is applied.

In other forms parts of the headgear may be removed subsequently to the fusing of the panels, either by a die cutting or any other suitable method.

In forms where there is a fused periphery of one or more lapped regions and the removal of parts of the headgear occurs along with or subsequently to the step of fusing the panels, the removal of material only within fused zones may maintain the fused periphery of the lapped region or regions.

Parts which are removed from the headgear may be of any suitable size and shape.

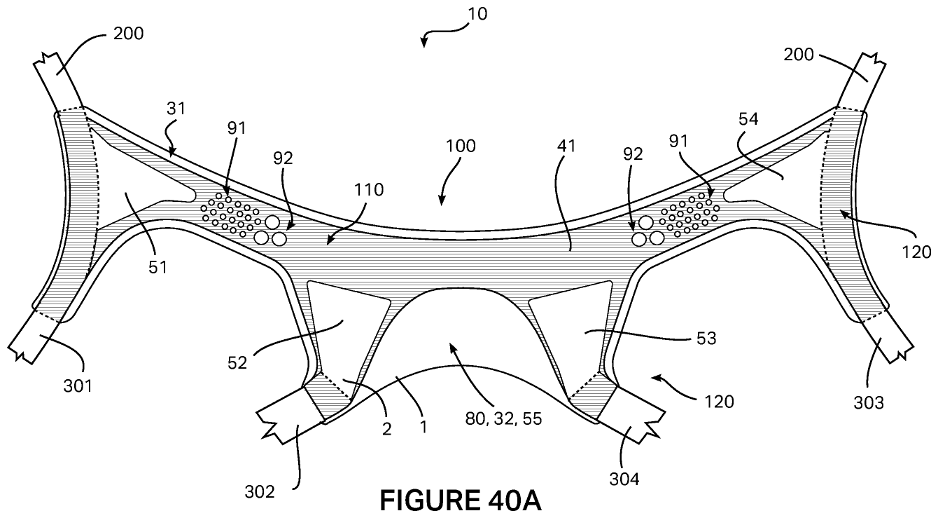

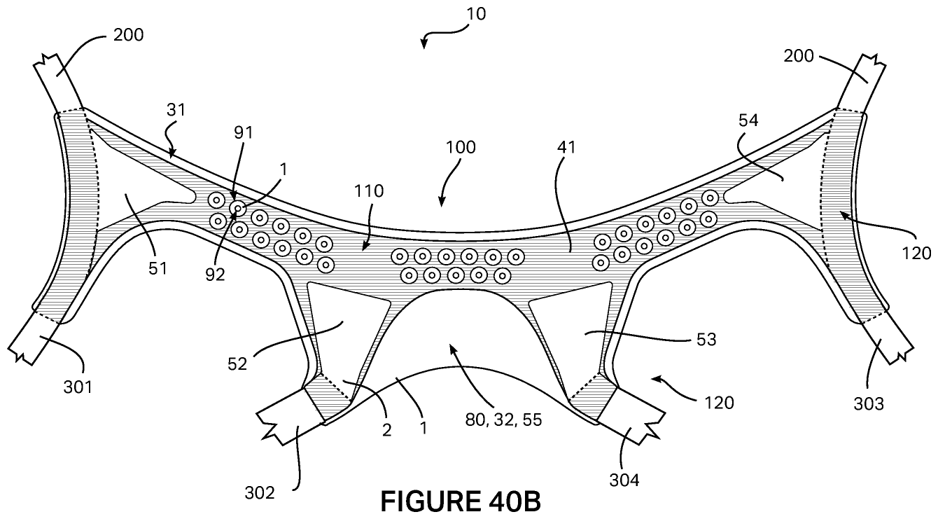

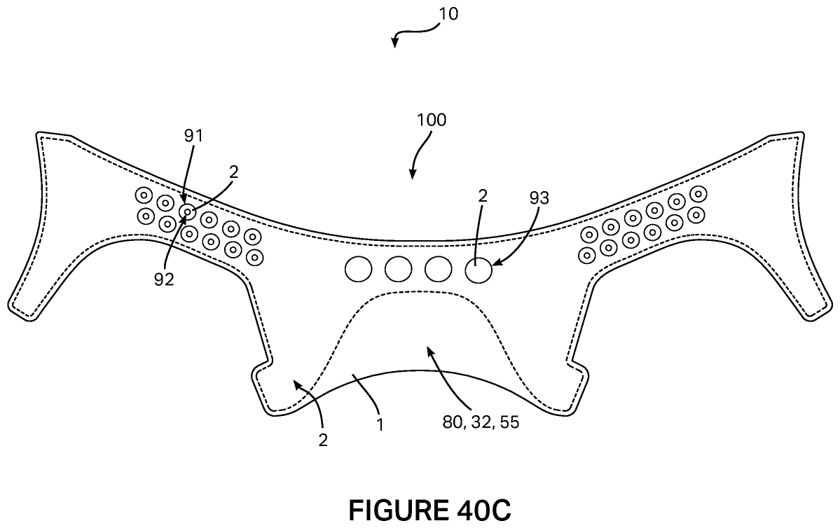

FIG. 40A shows the headgear 10 of FIG. 17B which has been joined with a plurality of straps 301-304 and 200. The headgear 10 of FIG. 40A illustrates how the headgear may be stamped through both the first layer 1 and second layer 2.

As seen in FIG. 40A, the headgear includes a cluster of first cut-outs 91 of a first size, and a cluster of second cut-outs 92 of a second, greater, size.

The cut-outs 91 and 92 of FIG. 40A pass through both the first panel 1 and second panel 2, to define a cut-out through the whole of the headgear.

Such cut-outs may reduce the weight of the headgear. They may also increase the breathability of the headgear.

The cut-outs 91 and 92 of FIG. 40A may be provided to each of the first panel and second panel individually before they are fused.

The cut-outs 92 and 92 of FIG. 40A may be provided to the first panel and second panel together, either before they are fused, as part of the fusing process, or after they are fused.

While FIG. 40A illustrates the cut-outs at lateral parts of the band 110, it will be appreciated that the cut-outs may be provided in any desired location on the headgear, and particularly where there is a desire to increase breathability of the headgear. Similarly, it will be appreciated that single cut-outs or clusters of cut-outs may be provided in any number of desired sizes and groupings.

FIG. 40B shows the headgear of FIG. 40A, but with a different configuration of cut-outs. As seen in FIG. 40A the headgear has a plurality of first cut-outs 91 of the second layer 2. Within the first cut-outs 91 and of a relatively smaller size, there are then respective second cut-outs 92 of the underlapped first panel 1.

This configuration may provide for increased reductions in weight relative to through holes of the size of the smaller cut-outs 92.

Where the first panel 1 is a more stretchable than the second panel 2, the configuration of FIG. 40B may also provide for localised regions of increased stretch within each cut-out 91 of the second panel 2.

While the larger cut-outs may be formed in the relevant panel before the panels are lapped with each other, the smaller cut-outs that pass through both panels may be formed once the panels have been lapped with each other, either before, during, or following the fusing of the panels.

FIG. 40C shows another embodiment of a headgear with cut-outs. FIG. 40C shows the rear portion 100 of FIG. 13, but from an opposite side. For example, where FIG. 13 shows a view of the outside of the headgear, FIG. 40C shows the patient-facing inside of the headgear.

In FIG. 40C the extent of the underlapped second panel 2 is shown in dashed lines.

As seen in FIG. 40C there are a plurality of first cut-outs 91 of the first panel. Within these and of a smaller size are second cut-outs 92 of the second panel 2. This is the opposite of the configuration of FIG. 40B.

In FIG. 40C the cut-outs 92 each define a hole through the headgear.

Also shown in FIG. 40C are a number of third cut-outs 93. These are cut-outs of the first layer 1, so that a region of the second layer 2 is exposed but no hole is defined through the headgear.

A headgear according to the disclosure may provide for one or more stretch zones 80. At a stretch region the headgear may provide for a relatively greater amount of extensibility than at adjacent parts of the headgear. The shape, size, and location of such stretch regions may allow for customised functionality of the headgear. For example, the stretch regions may be oriented to allow additional extensibility of the headgear in one direction, but not in another. The stretch regions may be configured to induce a relative rotation in parts of the headgear either side of the stretch region when there is a load applied across the stretch region.

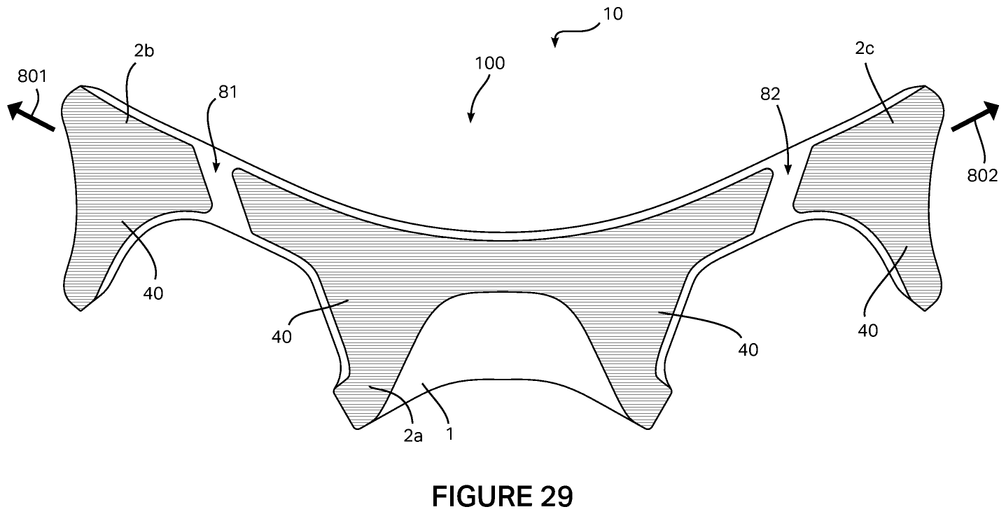

FIG. 29 shows an embodiment of a headgear fused from a first panel and second panel 2a, third panel 2b, and fourth panel 2c which are lapped to the first panel. Each of the second, third, and fourth panels may be of the same material or may alternatively be formed from two or three different materials.

The third panel 2b and fourth panel 2c are located on the first panel 1 spaced laterally apart from the second panel 2a. The gaps between these panels define a first and second stretch zones 81 and 82.

At the stretch zones 81 and 82 the headgear consists only of the first panel 1. As described in various other embodiments the first panel may be of a material which is relatively more extensible than the second (and third and fourth) panels, or at least which is just more extensible at its non-lapped regions where the stretch zones are than at the lapped regions where the panels have been fused.

As seen in FIG. 29 the stretch zones 81 and 82 are of substantially continuous lateral size and extend across the whole height of the band of the headgear.

When a load is applied across the headgear in the direction of the arrows 801 and 802 the headgear may stretch and extend in that direction due at least in part to extension at the stretch zones 81 and 82.

It may be desirable to provide for different amounts of extensibility in different parts of the headgear, to allow the headgear to stretch to a different shape than its laid-flat shape.

For example, it may be desirable that then the headgear is stretched an upper part of the headgear extends relatively more than a lower part of the headgear.

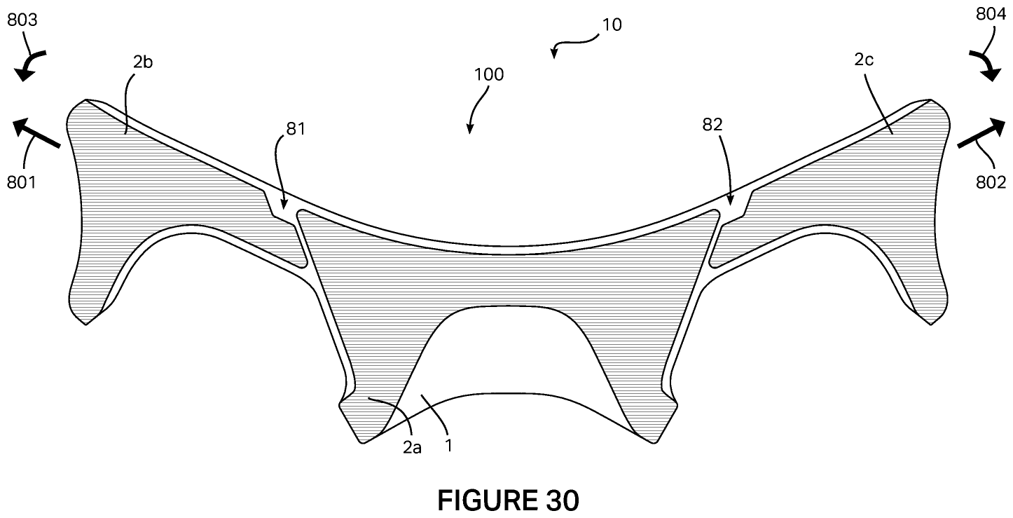

FIG. 30 illustrates an embodiment of the headgear 10 where two stretch zones 81 and 82 are configured to allow greater stretch towards a top of the band of the headgear than towards its bottom.

As seen in FIG. 30 the stretch zones 80 and 81 are of greater width towards the top of the band than at the bottom. Accordingly, when loads are applied to the direction of the arrows 801 and 802 the lateral ends of the headgear beyond the stretch zones 81 and 82 may be rotated at least some amount in the direction of the arrows 803 and 804.

Allowing more stretch at one part of the headgear 10 may be desirable to facilitate particular movements of the patient. For example, when a patient wearing the headgear lowers or raises their head, the distance between the top of the rear portion of the headgear and top attachment point to the patient interface and the distance between the bottom of the rear portion of the headgear and lower attachment point to the patient interface may change unequally. Accordingly, a greater stretchability at the top or bottom of the headgear may be desirable.

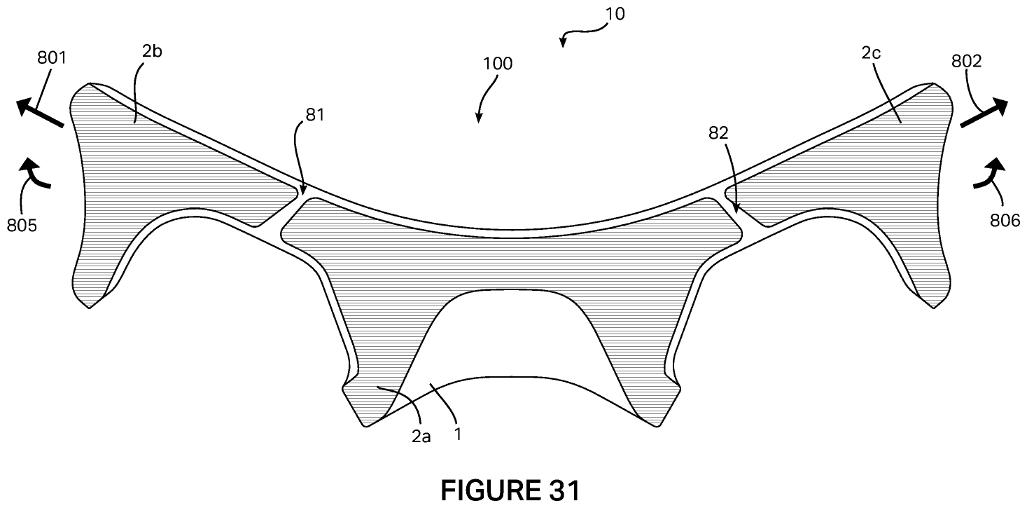

While FIG. 30 illustrates a configuration where the stretch zones have a stepped size along the lateral direction of the band, stretch zones may be provided that additionally or alternatively continuously vary in size along the lateral direction of the band. An example of this is shown in FIG. 31, where the stretch zones 81 and 82 are relatively narrower towards an upper part and relatively wider towards a lower part of the band of the headgear.

As the stretch regions are wider at their lower parts, when a load is applied in the direction of the arrows 801 and 802 the lateral parts of the headgear may be induced to rotate at least somewhat in the direction of the arrows 805 and 806.

An allowable amount of stretch of the headgear may be controlled by varying the size of the stretch zones and the stretch properties of the first panel 1.

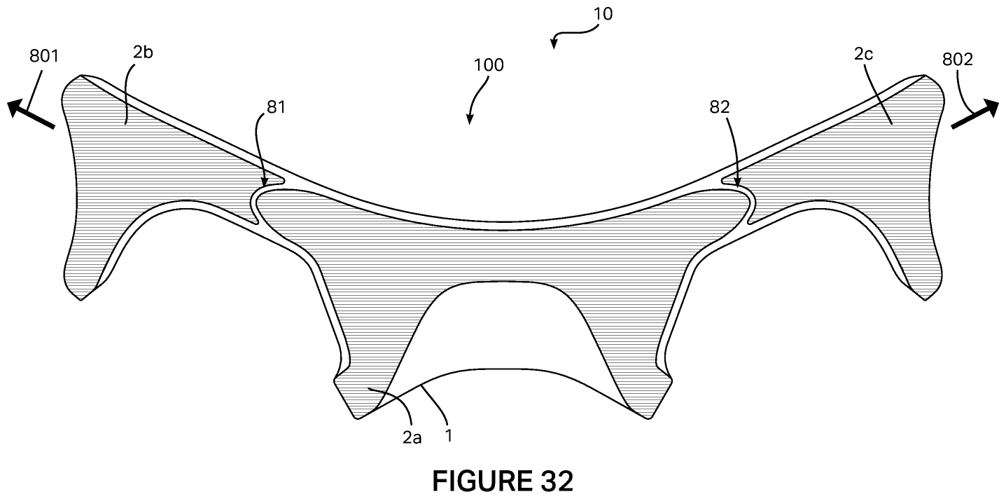

Stretch zones may also be provided in curved shapes, such as is illustrated by the stretch zones 81 and 82 of FIG. 32.

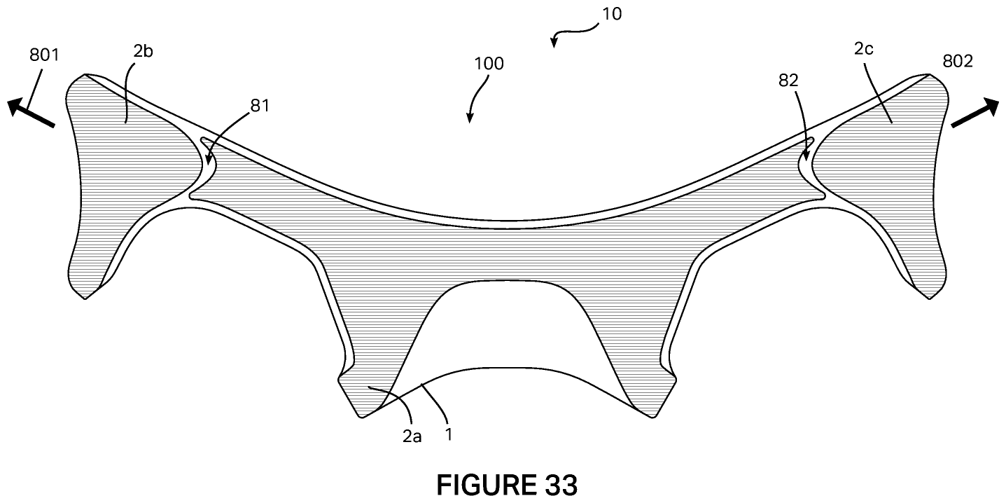

FIG. 33 illustrates a further embodiment where the curved stretch zones are oriented oppositely to that of FIG. 33, with the stretch zones 81 and 82 curving in towards the centre of the headgear.

In FIG. 33, unlike in FIG. 32, the stretch zones 81 and 82 are also of larger lateral dimension towards their centre than at their upper and lower ends. This may allow a relatively greater degree of stretch at the middle of the stretch zones 81 and 82.

While the foregoing examples have shown stretch zones which extend vertically across a whole height of part of the headgear to allow lateral stretch of the headgear, stretch zones may also be arranged laterally across the headgear to allow vertical stretch of the headgear.

For example, a stretch zone may be provided laterally across the fused zone of a headgear adjacent to a lower strap connection portion. This may allow additional vertical stretch in the headgear when load is applied to a connected strap.

A headgear may include one or more stretch zones. As illustrated in the configurations of FIGS. 29-33, these stretch zones may bisect the fused parts of the headgear, resulting in multiple separate fused zones.

In other configurations however, stretch zones may be provided that do not bisect the fused parts of the headgear, so that the fused zone of the headgear is one contiguous region.

Regardless of whether or not a headgear includes one or more stretch zones, the headgear may have one or more contiguous fused zones between two or more strap connection portions of the rear portion of the headgear. As loads are transferred between the straps of the headgear in use, a contiguous fused zone between two strap connection portions may aid in effectively transferring loads through the headgear.

For example, the rear portion 100 of FIG. 14 has a contiguous fused area of the first and second panels within the fused zone 41 between the lateral strap connection portions 120a and 120d. In fact, in the configuration of FIG. 14, the rear portion 100 defines contiguous regions of the fused zone 41 between each of the strap connection portions 120a-d.

The panels of the headgear may be configured to give a particular overall shape or structure to the headgear, which it may have when it is not being worn. This shape may be created by an existing 3D shape of the panels before they are fused, or by the fusing of the panels, or both.

Such shapes or structures may be important for signalling to a patient or person who will apply the headgear to a patient the nature of the various parts, and their intended orientations for donning. For example, it may be desirable that the headgear at rest presents an opening between the straps of each side within which the patient's head may be received. Similarly, it may be desirable that the headgear at rest presents the side straps as distinct members, projecting away from the remainder of the headgear and potentially also individually of each other, to allow easy identification and grasping of each strap by the patient so that they may be connected to the patient interface.

This shape and structure may for instance be demonstrated when the headgear 10 is held by a patient, or particularly when a portion of the headgear is grasped by the patient and the remainder of the headgear drapes or hangs from the grasped portion.

Where a headgear or part of a headgear includes two or more lapped panels, an edge of one or more of the panels may extend past an adjacent edge of one or more of the other lapped panels. An example of this was previously described in relation to FIG. 13, where the first panel 1 beyond the second panel 2 is unlapped and unsupported by the second panel 2. When worn, this configuration may allow contact pressure on the patient's head to gradually reduce from the edge of the lapped parts of first panel 1 and second panel 1 to the distal most unlapped part of the first panel 1. This may provide an edge softening effect for a patient when the headgear is worn.

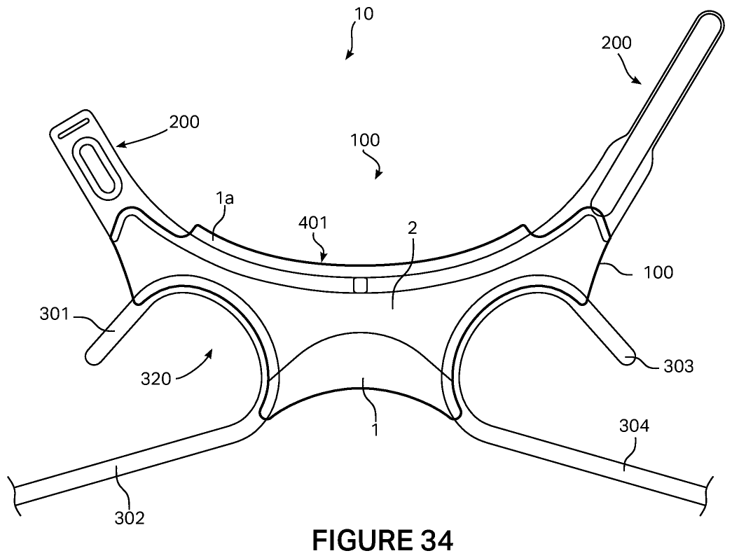

FIG. 34 illustrates a headgear 10 with such edge softening properties along at least part of an upper edge of the rear portion. The rear portion 100 of the headgear 10 includes a first panel 1 and second panel 2. A portion 1a first panel 1 at the rear portion 100 extends past the upper edge of the second panel 2. The portion 1a of the first panel 1 not lapped by the second panel 2. By this configuration, the first panel 1 defines an upper edge 401 of the rear portion 100 of the headgear around at least part of the rear of the patient's head.

As seen in FIG. 34, a top strap 200 is attached to the rear portion 100, and the portion 1a of the first panel 1 also extends beyond the upper edge of the top strap 200.

The overhanging portion 1a of the first panel 1 may provide an edge softening effect for a patient when the headgear is worn.

As illustrated in FIG. 34, the first panel 1 including the overhanging portion 1a is provided to be inside of the top strap 200 relative to the patient's head. As such, the overhanging portion 1a will roll away from the patient's head and preferably over the top strap 200 when the headgear is worn.

A panel which has an unlapped portion peripheral portion may be at least one of thinner, softer, and less dense than the headgear panel or part which it overlies, for example in the case of FIG. 34, the top strap 200.

While described in relation to forming a pressure-graduating edge at the upper side of the rear portion 100 of the headgear, the described techniques may be applied to any other part of the headgear to achieve the same functionality. For example, this panel configuration may be utilised at the lower periphery of the rear portion 100, and/or at the ear loops 320.

The panels of the headgear may be configured to give a particular overall shape or structure to the headgear, which it may have when it is not being worn.

Such shapes or structures may be important for signalling to a patient or person who will apply the headgear to a patient the nature of the various parts, and their intended orientations for donning. For example, it may be desirable that the headgear at rest presents an opening between the straps of each side within which the patient's head may be received. Similarly, it may be desirable that the headgear at rest presents the side straps as distinct members, projecting away from the remainder of the headgear and potentially also individually of each other, to allow easy identification and grasping of each strap by the patient so that they may be connected to the interface.

This shape and structure may for instance be demonstrated when the headgear 10 is held by a patient, or particularly when a portion of the headgear is grasped by the patient and the remainder of the headgear drapes or hangs from the grasped portion.

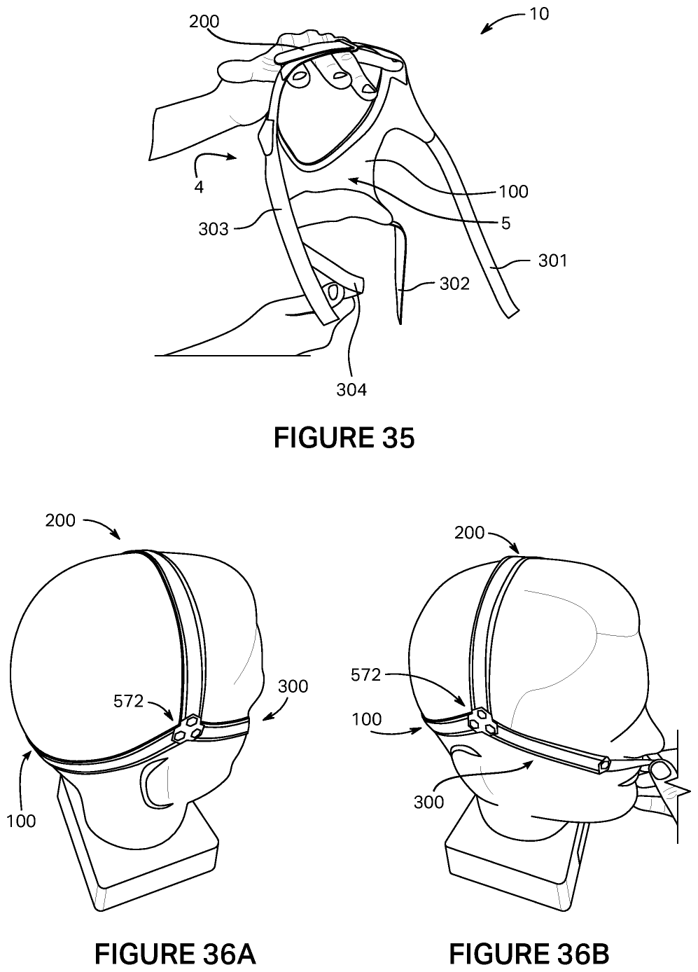

FIG. 35 illustrates a headgear 10 that is configured to display certain shape and form when not worn. The headgear 10 may have a top strap 200 which has sufficient stiffness that it at rest retains an at least partially circular form. This is shown in the example of FIG. 35, where the remainder of the top strap 200 draping from the patient's hand demonstrates a substantially circular form.

Also seen in FIG. 35 is the way in which the straps 300 can project away from the remainder of the headgear 10, and also discretely of each other. To provide such a configuration the panel or panels which comprise the straps 300 may be provided of a material of sufficient stiffness so as to drape with a sufficiently low amount of curvature over their length. In addition or alternatively, either or both of the rear portion 100 and/or top strap 200 to which the straps 300 attach may bias the straps to fall outwardly, away from each other and away from the remainder of the headgear 10.

To achieve such a configuration the straps 300 and/or the panels of the remainder of the headgear to which they lap may be provided with a natural curl or curve to them.

In some embodiments, a base panel may be lapped with a more peripheral stretch panel which is pre-stretched when fused to the base panel. This may induce a natural curvature in the fused panels, to provide a desired natural shape and/or divergence away from each other of the straps 300.

In addition or alternatively to any panel configurations to provide a resting shape to the headgear to signal the nature of the different portions or their use or application to a patient, the headgear may be configured to visually or by tactile cues signal these things to a patient.

For example, with reference to the headgear 10 of FIG. 35, the top strap and rear portion of the headgear may be provided of one or predominantly one colour. In order to be easily differentiated from the remainder of the headgear, one or more of the straps 300 may be provided having a different colour and/or texture than that of the top strap 200. As seen in FIG. 35, the upper side straps 301 and 303 may be provided having a different colour and/or texture than that of the lower side straps 302 and 304. Any such structural, visual, and/or tactile cues which may be built into the headgear may additionally or alternatively be utilised to aid a patient in differentiating between an internal surface 5 and an external surface 4 of the headgear 10.

For example, some or all of the panels which comprise the inner surface may be provided having a different colour or different colours than some or all of the panels which comprise the external surface 4 of the headgear.

In other configurations, some or all of the panels which comprise the internal surface 5 of the headgear may have a different texture than some or all of the panels defining the external surface 4. In particular, the texture of the inner surface 5 may be in part or whole softer than the external surface 4, to aid in signalling to the patient that this part should be in proximity to their head as well as to provide enhanced comfort when worn.

As seen in FIGS. 36A and 36B, the location of a join 572 between the rear portion 100, top strap 200, and side straps 300 may be configured to sit above the ear of the patient.

The side straps 300 of a headgear 10 may include one or more strap panels which are fused together.

The ends of the side straps of the headgear may pass through a fixture on the respiratory interface and fold back onto and fasten to themselves. To enable this there may be a two-part fastener system provided on the side strap, such as a hook-and-loop fastener.

However, when tightening a side strap back against itself it may be desirable to provide some form of feedback to the patient as to how tight the mask is and/or to provide some regular steps to which the straps may be adjusted to aid in easily equalising the amount that different straps are tightened.

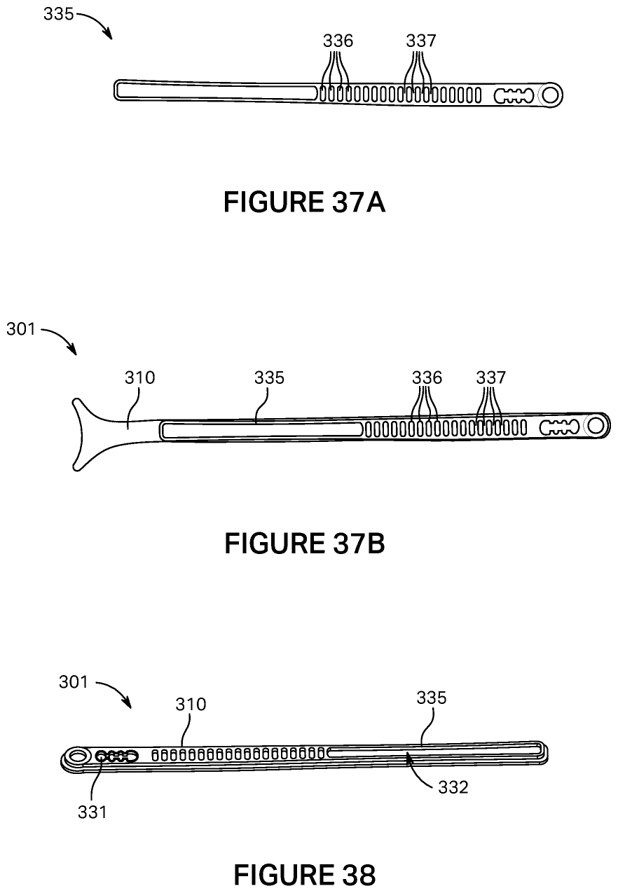

FIG. 37A shows an index panel 335 which is to be fused onto a base panel 310 to form a side strap. The index panel 335 includes at least a series of step-like formations 337 which extend at least partially between the two sides of the index panel 335.

The index panel 335 may comprise a material which is relatively stronger, stiffer, denser, and/or harder than the underlying base panel 310 to which it is to be fused.

FIG. 37B illustrates the index panel 335 of FIG. 37A fused to a base panel 310.

By providing the index panel 335 on the external surface 15 of the headgear relative to the base panel 310, the index panel 335 may come in contact with a fixture of the respiratory interface as the strap end is passed through it and pulled back towards the rear portion 100 of the headgear.

When there is tension on the two sides of the strap 301 across the fixture of the respiratory mask, the step formations 337 of the index panel 335 may provide tactile feedback to the patient as the straps are tightened on the respiratory interface.

A further function of such an index panel 335 which is of a relatively stronger, stiffer, denser, and/or harder material than the base panel 310 may be to provide wear resistance to the strap 301 as it engages with and passes over the fixture of the respiratory interface. For this purpose alone, a strap may include an index panel 335 without the defined cut-outs 336 to provide tactile feedback but may instead have an index panel 335 without cut-outs.

FIG. 38 illustrates a further configuration of a side strap 301 which includes a base panel 310 and an index panel 335 fused to it.

As seen in FIG. 38, a distal portion of the strap 301 has within cut-outs of the index panel 335 a first part 331 of a fastener system which corresponds with second part 332 of a fastener system. The second part 332 may be provided by the material of the base panel 310 itself, or by another material fused thereto.

While shown in FIGS. 37A-B and 38 as comprising a unitary panel with a plurality of cut-outs 336 to define the step formations 337, according to other embodiments the index panel 335 may be provided just as the step formations 337. In other words, the index panel 335 may instead comprise a plurality of step formations 337 without any linking material to define a single unitary panel.

The step formations 337 may present a raised surface above the surface of an underlying strap panel, such that the step formations provide resistance to movement of the strap relative to the interface. The spacing of the step formations may provide for an indication of indexing of each strap by a patient as they adjust the tension on the headgear straps.

To provide the indexed resistance the step formations 337 may be of a different and potentially harder or denser material than that of the underlying strap panel.

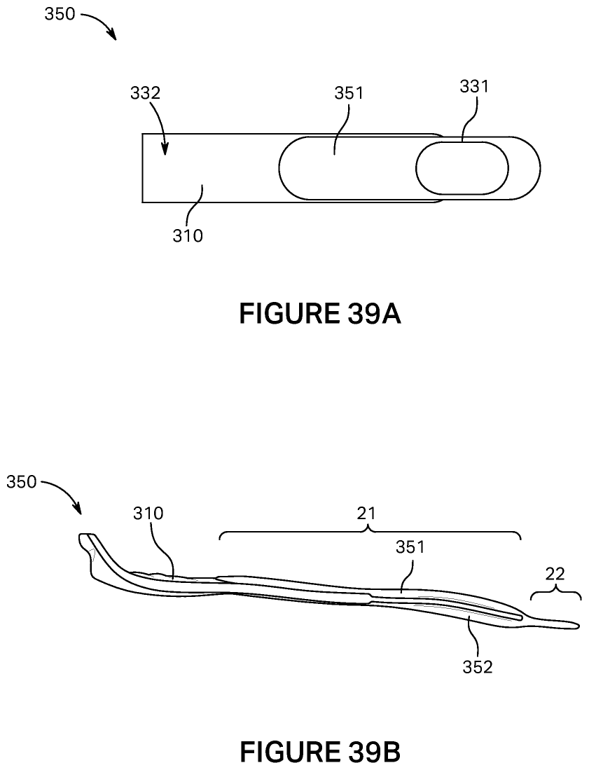

An example of such a strap-end feature 350 is illustrated in FIGS. 39A and 39B. As seen in FIG. 39A, the strap-end feature 350 includes a first tab panel 351 which is at least partially lapped to the strap panel 310. As seen in FIG. 39A, the first tab panel 351 has a greater surface smoothness than that of the strap panel 310, and thus may be able to be distinguished from it by touch.

On top of the first tab panel 351 is a first part 331 of a hook and loop fastener. In some embodiments the outer surface of the strap panel 310 may form the second part 332 of the hook and loop fastener, to engage the strap end with the strap when the strap end is folded back onto itself.

As seen in the configuration of FIG. 39A the strap panel 310 comprises the second half 332 of the hook and loop fastener. For example, the strap panel 310 may be of an unbroken loop material, and loops presented at the surface of the strap panel 310 may be utilised as the loops of the hook and loop fastener.

The strap panel 310 may be singly lapped by the first tab panel 351, or it may be lapped on its other major face by a corresponding second tab panel 352, as is illustrated in the side view of FIG. 39B.

While the presence of a first and/or second tab panel 351 and 352 may provide a desired degree of ability to differentiate between the strap end and the remainder of the strap, adhesive may be utilised to enhance this differentiation. For example, the adhesive may provide the strap end feature 350 with an increased stiffness relative to the strap panel 310. This difference in stiffness may act the or an additional physical cue to the patient as to the presence of the strap end.

The first and second tab panels 351 and 352 may be fully lapped against the strap panel 310 to provide a three-panel layup, such as is shown at the first lapped region 21 of FIG. 39B.

In other configurations the tab panels 351 and 352 may extend distally of the end of the strap panel 310, as is seen in FIG. 39B, to form a second lapped region 22.

The difference in thickness between the first lapped region 21 and second lapped region 22 may provide a further tactile cue for patient as to the location of the end of a strap.

Where the tab panels 351 and 352 comprise a second lapped region 22 at which they only singly lap each other, rather than lap either sides of the strap panel 310, an additional amount, or different type, of adhesive may be utilised at the second lapped region 22. This may provide the second lapped region 22 with an increased stiffness relative to that of either the first lapped region 21 or the strap panel 310 by itself and may be another potential form of cue to a patient that they are grasping the tab ends.

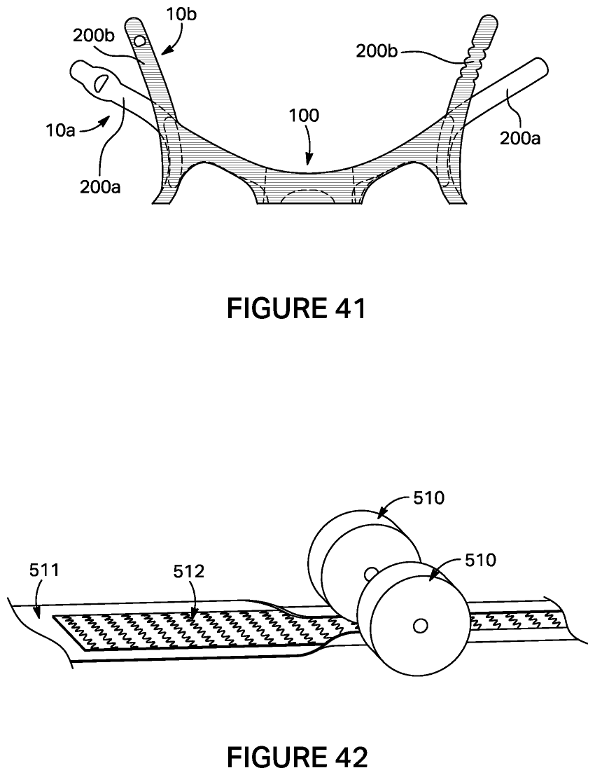

FIG. 41 is a view of the upper portions of two different configurations 10a and 10b of a headgear overlaid on each other. The two headgear each have a different configuration of their top straps 200.

The first configuration 10a has top straps 200a and the second configuration 10b has top straps 200b. The top straps 200b are oriented so they project less laterally outwards than do the top straps 200a, and instead extend primarily upwards. In FIG. 41 the top straps 200a are oriented so that they are substantially continuous extensions of the adjacent parts of the rear portion 100.

As seen in FIG. 41 the top straps 200a extend laterally outwards at angles of approximately 35 degrees to horizontal. The second configuration with the top straps 200b project at a steeper angle to the horizontal of approximately 70 degrees to the horizontal.

The angle of the top straps 200 of a headgear relative to a lateral axis of the headgear may influence where the top straps are located, in use, on the head of the patient. In some configurations it may be desirable for the top straps to pass, in use, vertically and laterally across the patient's head, with minimal or no proximal or distal displacement between the bases and ends of the top straps.

Regardless of where the straps pass across the patient's head, it may be desirable to select the angle of the top straps to the lateral axis of the headgear such that the top straps lie flat against the patient's head to improve comfort.

In use the more steeply oriented top straps 200b of FIG. 41 will cross the patient's head further forwards than would the more shallowly oriented top straps 200a.

The manufacture of a headgear according to the disclosure may involve the fusing of part or all of the headgear. While various discrete fusing processes are elsewhere described herein, such as with the welding press of FIGS. 9A and 9B, fusing may additionally or alternatively be provided as a continuous process.

For example, FIG. 42 illustrates a continuous welding operation where two rollers 510 are used to continuously weld two panels 511 and 512 together. Such a process may be desirable for increasing a speed of manufacture and may be particularly suited to continuous shapes such as welding lengths of panels which can be subsequently cut into sections for use as headgear straps.

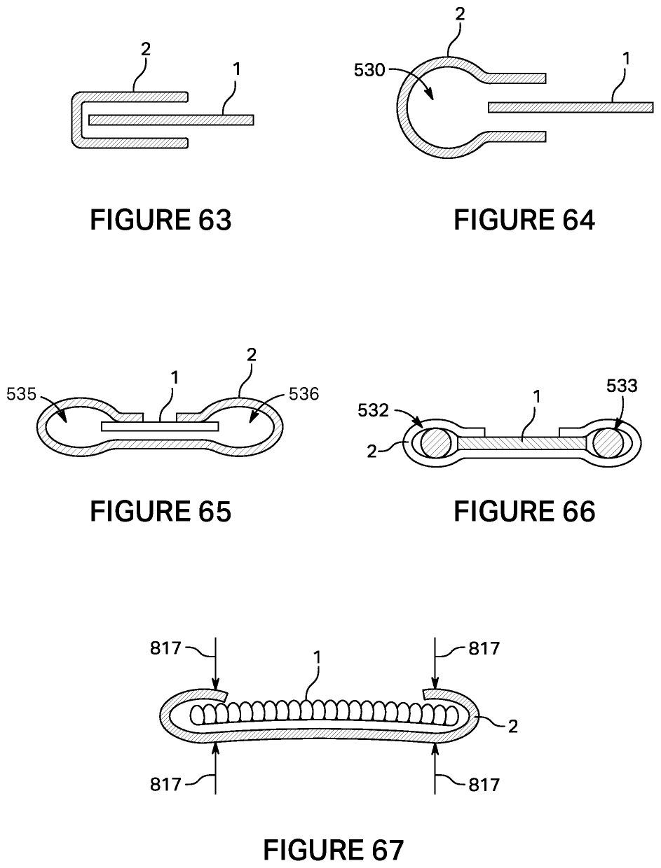

A continuous welding process may also be used to form a folded edge or edges of a headgear, such as is subsequently described in further detail for example in relation FIGS. 63-67. An example of a continuous fusing operation to form a headgear portion with folded edges is illustrated in FIG. 42, where the rollers 510 are used to continuously fuse the first panel 511 and second panel 512 together, along the edges of the first panel 511 each of which are folded back onto the second panel 512.

While a continuous welding process may be applied to a panel or panels of a continuous width, the width of the panels may vary.

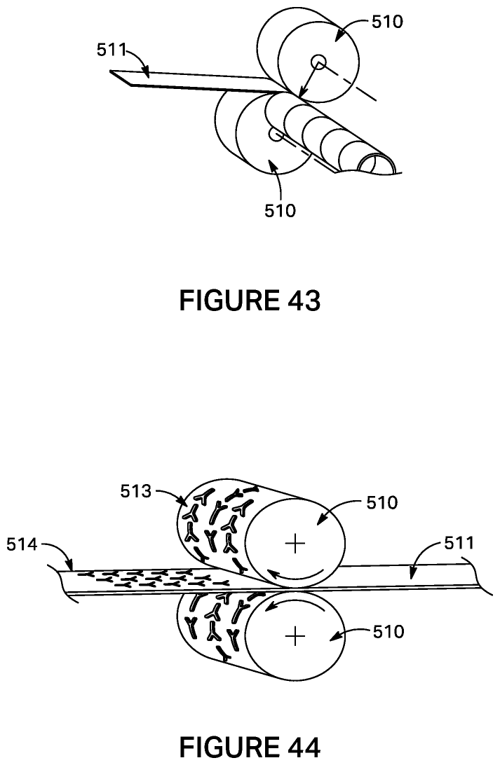

While FIG. 42 illustrates a continuous welding process performed in a plane, a continuous welding operation may be performed in other configurations such as where one or more panels are concentrically wound, and the helical joins are welded. An example of this is illustrated in FIG. 43, where a panel 511 is wrapped into a tube shape and rollers 510 weld at least the overlapping portions to fuse them together and hold the panel 511 in its tube shape.

A headgear according to the disclosure may be fused using one or more different fusing methods. For example, fusing may be provided by one or both of discrete and continuous processes. Fusing may be applied using only one type of fusing process, such as a high frequency welding, or it multiple different processes such as both a high frequency welding and a direct thermal welding may be applied to the same or different parts of the headgear.

Continuous welding processes may also be used to define fused and non-fused zones of the panels between the rollers, or to provide varying degrees of fusing, by using debossed regions of welding roller, in a similar configuration as described in relation to the die of FIG. 20.

FIG. 44 is an example of a continuous welding process where two rollers 510 each include a pattern of debossed regions 513, such that the panel 511 when welded are provided with a corresponding pattern of non-fused zones 514.A panel or panels which are to be fused may be pre-stretched before they are fused. Pre-stretching in combination with fusing may be used to cause a planar panel to assume a 3D shape. For example, a panel may be stretched then only one part of it fused. When the tension is released, the fused portion may recover less extension than the non-fused portions. This may urge the panel to assume a 3D shape.

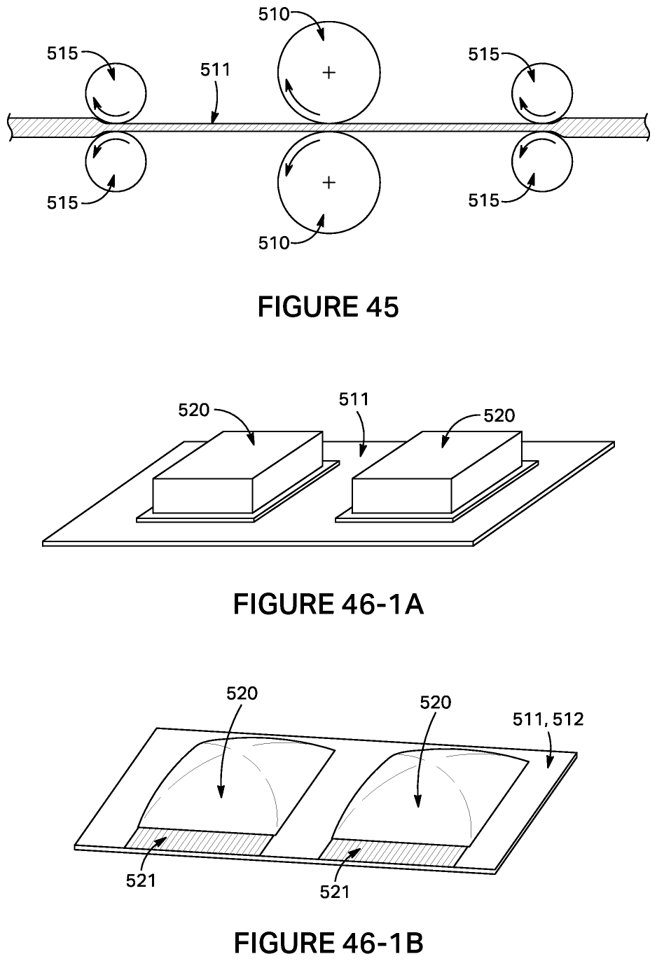

Pre-stretching may be utilised in either discrete or continuous fusing processes. FIG. 45 illustrates a continuous welding process with two rollers 510 which each act as a welding die to weld one or more panels 511, where the panels are pre-stretched by sets of secondary rollers 515.

A pre-stretched panel or set of panels may be lapped with another panel or set of panels which are not pre-stretched, and the lapped panels may then be welded together. The pre-stretching at one side of the set of lapped panels may be utilised to distort the panels into a desired 3D shape.

According to various configurations it may be desirable to form pockets or voids between two panels which are to be welded together. Such pockets or voids may be formed by selectively non-fusing the lapped region where the void is desired.

Where additional volume is desired in the pocket or void, and/or where it is desirable that the panels have fused material properties such as a surface finish or a stiffness at the pocket or void, one or more inserts 520 may be used in forming the pocket or void.

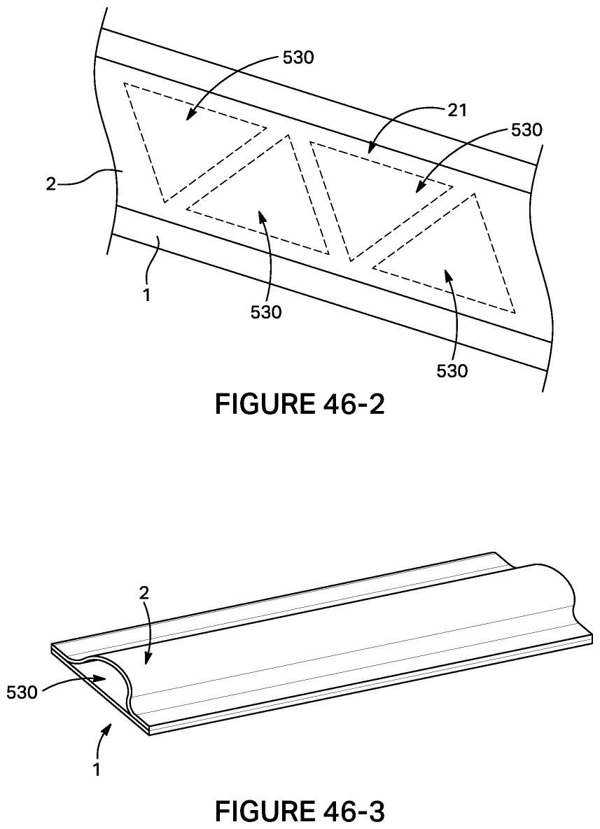

FIG. 46-1A illustrates two inserts 520 which are provided on top of a first panel 511. The inserts 520 shown have a rectangular cuboid shape, but it will be appreciated that they may be any desired shape as needed to impart the desired shape to the pocket or void.

In FIG. 46-1B a second panel 512 has been overlaid on the first panel 511, and the two panels have been fused together, then the inserts 520 have been removed through the open and non-fused sides 521.

The second panel may optionally be stretched over the inserts 520 before the panels are fused.

As seen in FIG. 46-1B the fused second panel 512 has retained a 3D shape to define two pockets 521.

Once the inserts 520 have been removed, the non-fused sides 521 may subsequently be fused or otherwise closed, if required, to close the pockets and form them into closed internal voids within the panels.

Where a fusing is applied across the inserts 520 they may be made of a non-fusible material. For example, in the case of high frequency welding, they may not include a dipolar material. Or, in the case of a direct thermal welding, they may have a melting point higher than the welding temperature.

FIG. 46-2 shows a first panel 1 which is partially lapped by a second panel 2. The lapped region 21 has been fused leave a series of voids 530 inside the lapped region 21 where the first panel 1 and second panel 2 are not fused together.

FIG. 46-3 shows another configuration where a first panel 1 and second panel 2 have been fused together along two opposite sides to leave a continuous pocket 530 running the length of the two lapped panels.

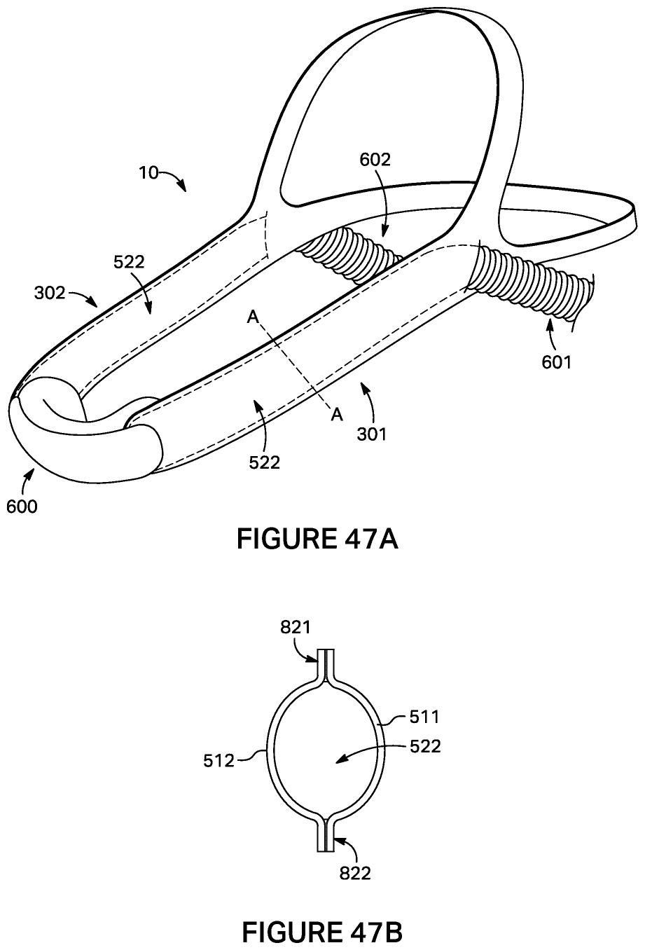

According to some embodiments of the disclosure, the fused panels of the headgear may be used to define air conduits. An example of such a configuration is shown in FIG. 47A, where a headgear 10 defines an air conduit 522 in each of its two side arms or side straps 301 and 302. The front portion of each side arm connects to a patient interface 600. External conduits 601 and 602 may then attach to more rearward portions of the headgear at the rear of each of the straps 301 and 302 as seen in FIG. 47A. In other forms the air conduits of the headgear may extend into the rear portion or top strap of the headgear, and the external conduit or conduits may attach at one or more locations on the rear portion or top strap of the headgear.

Air conduits may be formed in the headgear by selectively leaving portions of the headgear unfused. FIG. 47B illustrates a cross-sectional view through the line A-A of FIG. 47A. In FIG. 47B a first panel 511 and second panel 512 have been fused together along their edges 821 and 822, but the central portions have been left unfused to define the air conduit 522.

In addition to being left unfused the air conduits may be formed using inserts such as the inserts 520 described in relation to FIGS. 46-1A and 46-1B.

A pocket in a non-fused zone of the headgear may be accessible through a non-fused periphery of the lapped panels, as illustrated in FIG. 46B. A pocket may also be accessible through a cut-out of one of the lapped panels, where the periphery of the lapped panels are otherwise fully fused together.

Pockets of the headgear may be used to receive and hold inserts, such as stiffening inserts.

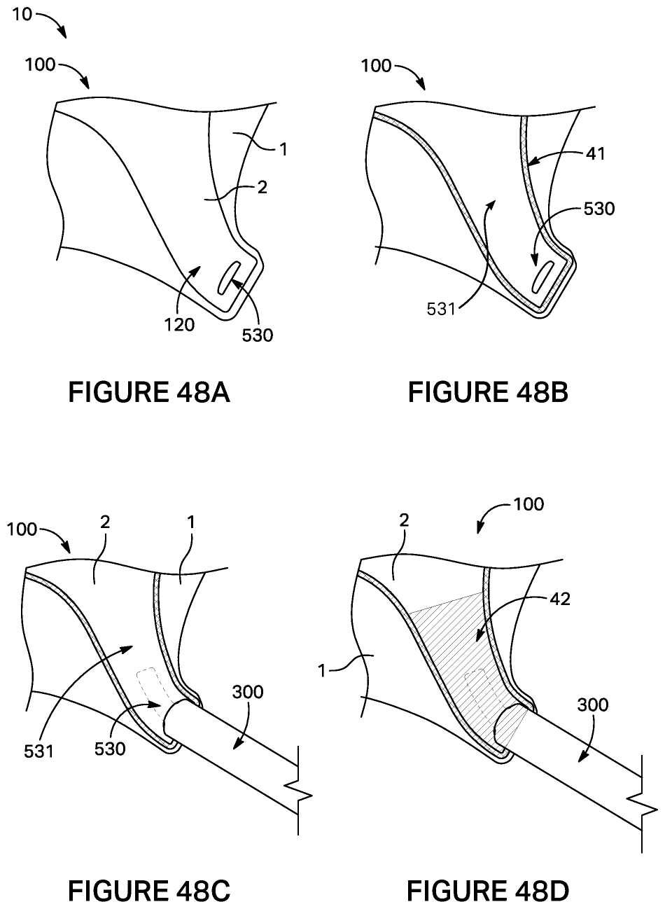

Pockets may additionally be utilised in the manufacture of the headgear, where it is assembled in multiple steps. For example, FIGS. 48A-D are steps in a process for attaching a strap to the remainder of the headgear by inserting the strap into a pocket of the headgear.

FIG. 48A illustrates part of a rear portion 100 of a headgear 10 that includes a strap connection portion 120. The rear portion 100 has a first panel 1 and a second panel 2 partially lapping the first panel 1. The second panel 2 includes a cut-out 530. In FIG. 48A the first panel 1 is visible through the cut-out 530 of the second panel 2.

The lapped first and second panels are then subsequently welded to each other about their periphery, as illustrated by the fused zone 41 of FIG. 48B. The fusing about the periphery of forms a pocket 531 between the first panel and second panel, which is accessible through the cut-out 530.

With the first and second panels joined to each other, the end of a strap 300 is inserted into the pocket 531 through the cut-out 530 as illustrated into 48C.

The strap end may be of the same size as the remainder of the strap, or, as illustrated in FIG. 48C, the strap end can be of a reduced size.

With the strap end inserted into the pocket 531, the pocket and strap end can be fused to secure the strap to the first and second panels. FIG. 48D illustrates the arrangement after the parts have been fused across part of the pocket, defining the fused zone 42.

Assembling a strap to the other panels of the headgear by insertion of one part into a pocket of the other may limit the potential for misalignment of the parts during assembly. For example, as in FIGS. 48A-D only the end portion of the strap 300 is sized to be received through the cut-out 530. Using a pocket to provide such physical constraints may simplify manufacture compared to other methods, such as joining a strap or other panel by sandwiching it between the loose ends of two other panels.

In an alternative configuration, a strap and rear portion could be joined as illustrated in FIGS. 48A-D, but without a cut-out 530 of the second panel 2 by leaving the strap-adjacent perimeter portion of the first panel 1 and second panel 2 unwelded in the second step. The strap 300 can then be inserted into the pocket 531 through the open perimeter, then the components welded together.

As previously described, selective fusing of different regions of a panel or multiple lapped panels may be employed to provide a desired characteristic to a headgear or part of a headgear. These characteristics may include texture, thickness, stretch properties, or surface properties such as UBL properties.

Such selective fusing may be carried out by regions, where entire parts of the headgear may be fused, and others left unfused. For example, as in FIG. 14 the entire lapped region 21 may be fused, while the entire unlapped region 32 is left unfused. Or, as in FIG. 48B-D the perimeter of a lapped region or part thereof may be fused, with the non-lapped region and internal portions of the lapped region left unfused.

Selective fusing may additionally or alternatively be carried out to provide areas of patterns of fused or non-fused area, such as have been described in relation to FIGS. 21 and 19, respectively.

As previously described, according to some configurations a headgear may at one or more locations satisfy the straight-line test, such that along a straight line drawn between two edges of the headgear, and more particularly between two edges of a lapped region of the headgear, there is more welded length than there is non-welded length.

FIG. 14 illustrates a rear portion 100 of a headgear which satisfies the straight-line condition along any straight line drawn between two edges of the lapped region 21.

While the lapped region 21 of the rear portion 100 of FIG. 14 is fully fused, the straight-line test may be satisfied in other configurations where a lapped region is not fully fused. For example, in the configuration of FIG. 19, the size and spacing of the fused zones 53 may be arranged such that along one or more lines drawn between locations along the upper and lower edges of the lapped region of the first and second panels the straight-lien condition may be satisfied.

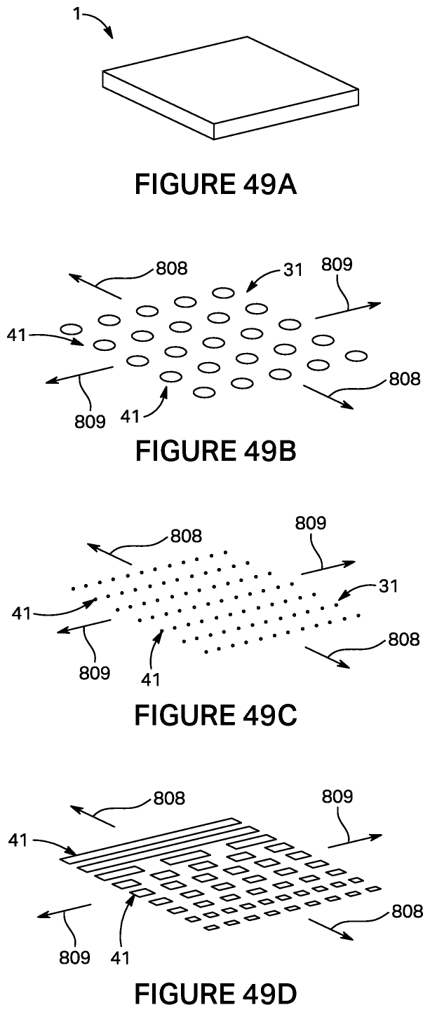

FIG. 49A is a view of an example panel 1, and FIGS. 49B-D show examples of different arrangements or patterns of fusing that may be provided to a panel or panels of the size of the example panel 1.

FIG. 49B illustrates an array of spaced circular fused zones 41, within a surrounding area which is unfused 31.

FIG. 49C illustrates the same array of fused zones 41 as in FIG. 49B, but where the fused zones are smaller and denser than the configuration of FIG. 49B.

The fused configurations of FIGS. 49B and 49C may reduce the stretch of a panel which is fused in this manner. However, the stretch properties in each of the direction of the arrows 808 and 809 in FIGS. 49B and 49C will be altered to the same degree, owing to the symmetrical arrangement of the fused zones in each direction.

In some configurations however it may be desirable to provide directionally altered stretch characteristics by selective fusing.

FIG. 49D shows another pattern in which fusing may be applied, where the density of fused area decreases in rows along the panel. Such a configuration may provide for uniform stretch characteristics across the panel in the direction of the arrows 808 when the panel is stretched in the direction of the arrows 809, but non-uniform stretch characteristics in the direction of the arrows 809 when the panel is stretched in the direction of the arrows 808. When stretched in the direction of the arrows 808 the degree of stretchability will increase towards the lower part of the panel as the density of fused area along the direction of stretching decreases.

Accordingly, by such configurations the directional stretch properties of a single panel, or a composite of panels, may be locally controlled. This may allow stretch to be provided in only areas where it is desired, in a direction that it is desired, and to the degree it is desired.

By selectively fusing the headgear the properties of a panel or area of lapped panels may be altered without the addition of other materials or fixtures. This may allow a headgear of highly customised and localised properties to be manufactured from a single panel of unitary properties, or lapped combinations of multiple panels, each of unitary properties.

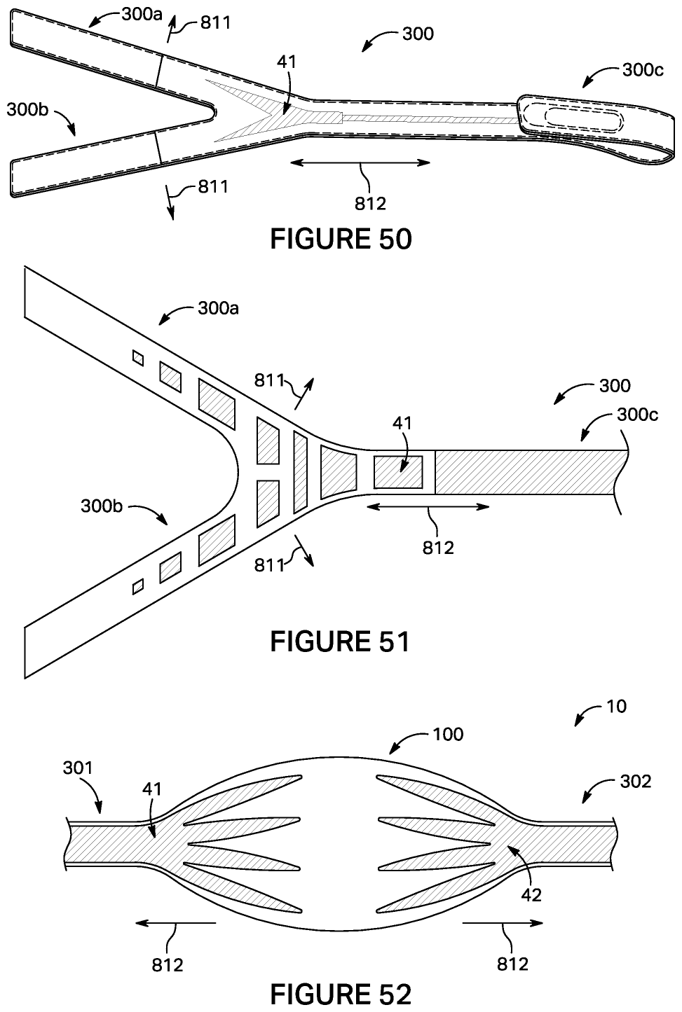

FIG. 50 illustrates a strap 300 of a headgear. The strap has a fused zone 41 which defines a bifurcated region towards where the strap splits into two parts 300a and 300b. The bifurcated nature of the fused zone 41 will allow the strap parts 300a and 300b to open away from each other in the direction of the arrows 811 but the contiguous fused zone from the split of the strap towards its end 300c along the direction of the arrows 812 will resist or prevent stretching in that direction.

FIG. 51 illustrates another similar configuration, but where the strap 300 is fused to define a first fused zone 41 towards the strap end 300c and then a decreasing amount of fused area towards the bifurcation of the strap and along each part 300a and 300b. This configuration will provide similar functionality as described in relation to FIG. 50, but the strap will have an increased amount of stretch in the direction of the arrows 812 at the region proximate to the bifurcation due to the non-contiguous nature of the fused zone 42 in that region along the direction of the arrows 812.

While described in relation to straps, it will be appreciated that the same concepts described in relation to FIGS. 50 and 51 could be applied to other portions of the headgear.

For example, FIG. 52 shows a rear portion 100 of a headgear 10 and proximate parts of two straps 301 and 302. The rear portion 100 and straps 301 and 302 may be formed from a single panel or combination of panels. As seen in FIG. 52, the rear portion 100 and straps 301 and 302 have been fused to define fused zones 41 and 42 in which the straps are fused along their length and fingers of fused area extend from each strap 301 and 302 into the rear portion. The size of each fused finger tapers towards its point.

Such a configuration may progressively limit stretch of the rear portion 100 towards its lateral sides in the direction of the arrows 812, while allowing stretch at the middle of the rear portion. The fused fingers may however have a less marked effect on stretch of the rear portion in the direction of the arrows 812, as the non-fused material between the fingers will allow stretch in that direction.

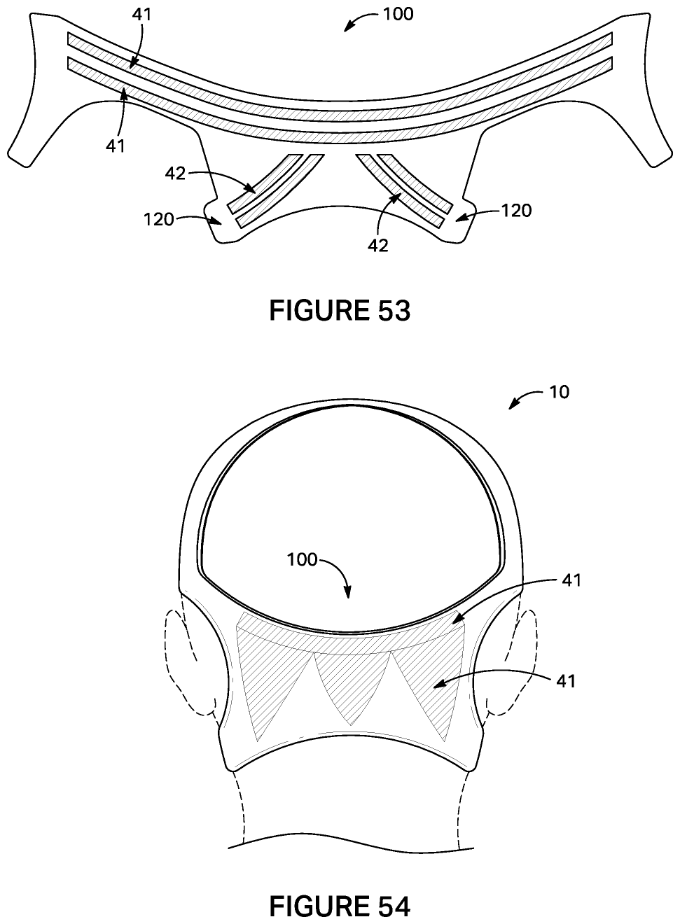

FIG. 53 shows a rear portion 100 which has fused zones 41 which extend continuously from one lateral side of the band 110 to the other, and fused zones 42 which extend in continuous lines from the strap connection portions 120 to the central part of the band 110. Such a configuration may provide for increased stiffness and ability for load transfer along the direction of the continuous parts of the fused zones 41 and 42, but still allow for stretch in the perpendicular direction. For example, the headgear may resist stretching along the lateral direction of the band more than across the height of the band 110.

FIG. 54 shows another headgear 10 when worn by a patient. The headgear 10 has a rear portion 100 which has a fused zone 41. The fused zone 41 comprises continuous part towards the top of the rear portion, then three triangular parts each of which taper towards the bottom of the rear portion.

Due to the configuration of the fused zone 41, this headgear 10 may limit lateral stretch towards the top of the rear portion, but increasingly allow stretch towards the bottom of the rear portion 100.

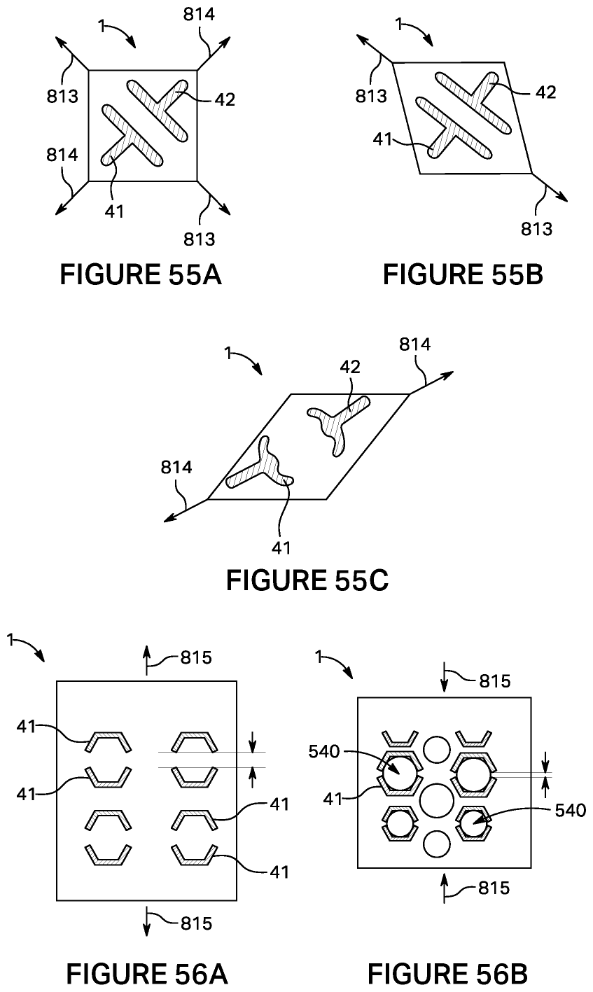

FIGS. 55A-C illustrate a further example of how a selective fusing of a panel may provide directionally different stretch properties.

As seen in FIG. 55A a panel 1 has been fused to define two fused zones 41 and 42. In the example of FIG. 55A the two fused zones are in mirror image “T” shapes.

The fused zones define a continuously fused part of the panel along the first diagonal of the panel in the direction of the arrows 813 but have a gap of non-fused panel between the two fused zones 41 and 42 along the other diagonal in the direction of the arrows 814.

While illustrated using “T” shaped fused zones, it will be appreciated that directional stiffness in the first lateral direction 813 relative to the second lateral direction 814 could be provided by the two parallel and adjacent parts of the “T” shapes which extend in the direction 813, without the legs that extend in the direction 814.

In the configuration of FIGS. 55A-C portions of each “T” shape extending in the direction of the arrows 814 may provide in-plane distortion of the panel when it is stretched in along the diagonal of the arrows 814. As seen in FIG. 55C, when stretched along the arrows 814 the arms of the “T” shapes that extend in the direction 813 may be bent towards each other.

While directionally different stretch properties are illustrated in FIGS. 55A-C using reflected “T”-shape fused zones, it will be appreciated the fused zones may be shaped and oriented so as to provide desired directional stretch properties or to produce other desired in-plane deformations under stretch.

It is a known issue that some full-face masks may tend to migrate upwards on a patient's face when they are worn. By using panels which have directionally different stretch characteristics, straps may be manufactured which are resistant to stretching in the direction of migration. Such a configuration may aid in preventing migration of the mask on the patient's face while maintaining stretch in other directions where it may still be desirable. For example, by employing selective fusing to provide directionally different stretch properties, headgear straps may be manufactured that are of reduced stretch transverse to their length, but not in a direction along their length.

Furthermore, it will be appreciated that by controlling the orientation of fused zones, such as the “T”-shaped fused zones 41 and 42 of the configuration of FIGS. 55A-C, it may be possible to locally reorient stretch and non-stretch directions of parts of the headgear. For example, different parts of a headgear strap may allow or resist stretch in different directions.

While illustrated in FIGS. 55A-C as using only two fused zones, it will be appreciated that patterns of similar small fused zones may be utilised across larger areas of a panel or panels.

Pre-stretching of panels may be combined with fusing to create surface features of the headgear.

An example of such a configuration is illustrated in FIGS. 56A and 56B. In FIG. 56A a panel 1 is shown which has been pre-stretched in the direction of the arrows 815 before being fused to define a pattern of fused zones 41-44. After fusing the stretch is removed, and the panel is allowed to contract in the direction of the arrows 816. As the fused parts move towards each other, they may cause bunching up of un-fused parts of the panel. This is illustrated in FIG. 56B, where the panel has recovered in the direction of the arrows 816 and bunchings 540 of the panel have been formed.

While fusing of panels may be used to alter material properties, such as stretch properties, to change the surface properties, or create surface features such as the bunchings 540, fusing may also be used to provide structure to a headgear.

As a degree of fusing increased, the fused panel or panels may become more plasticised and more rigid. Accordingly, fusing of a panel or panels may be used to provide structure to the headgear.

Any such additional structure provided by fused zones may allow for reduction in the size and bulk of the headgear.

Structure may be provided to the headgear by fusing of a panel, or by fusing another element such as a solid piece of plastic to the panel or panels of the headgear.

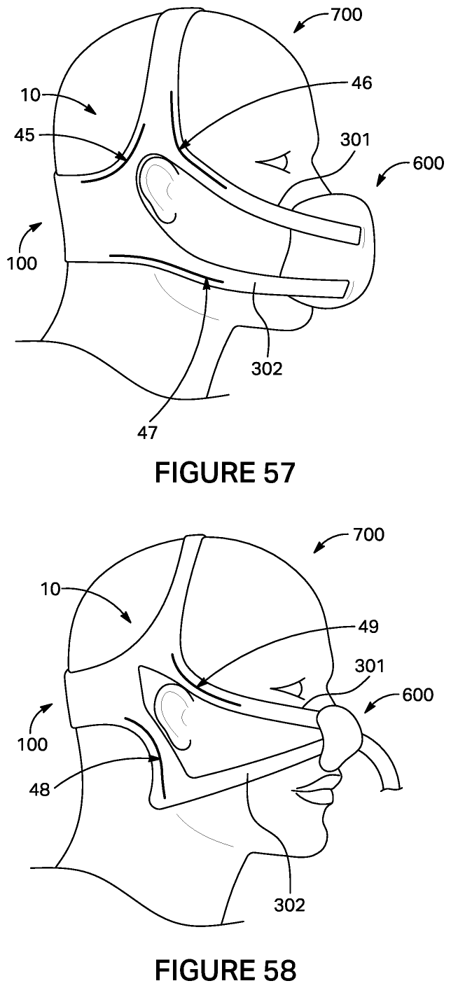

FIG. 57 shows a headgear 10 retaining an interface 600 to the face of a patient 700. The headgear 10 has three discrete fused structures 45-47. Each of the fused structures are relatively stiffer than the surrounding headgear, and act to transfer loads between different parts of the headgear while retaining a desired shape of the headgear.

The first fused structure 45 provides stiffness between the rear portion 100 and the top strap 200. The second fused structure 46 provides stiffness between the top strap 200 and the upper side strap 301. The third fused structure 47 provides stiffness between the rear portion 100 and the lower side strap 302.

The first fused structure 45 may aid in preventing upwards or downwards migration of the rear portion 100 on the patient's head. Similarly, each of the second fused structure 46 and third fused structure 47 may resist upwards or downwards movement of the straps, which will in turn prevent migration of the interface 600 up or down on the patient's face.

While illustrated in FIG. 57 as three discrete structures, in other configurations the three fused structures 45-47 could be provided as a single continuous structure.

The structure provided to the headgear by such fused structures may allow different configurations of the shape and positioning of the headgear on a patient's head. For example, they may allow the rear portion to be located higher up on the patient's head, away from their neck, than would otherwise be possible without causing distortion of the headgear.

FIG. 58 illustrates an embodiment of a headgear 10 retaining a nasal interface 600 on a patient's face. The headgear is configured so that the rear portion 100 sits higher up the back of the patient's head than the headgear 10 of FIG. 57.

Because of the higher position of the rear portion 100, the lower side strap 302 must extend down and around the patient's ear to avoid interfering with it. A first fused structure 48 provides stiffness to the rear portion 100 and lower side strap 302 around the rear of the patient's ear. Similarly, the upper side strap 301 does not connect in a direct line to the rear portion 100, so a second fused structure 49 provides stiffness between those two parts.

As illustrated in FIG. 58, in addition or alternatively to allowing repositioning of the rear portion 100 further up on the back of the patient's head, the use of fused structures may allow the headgear to be reconfigured to sit further away from the patient's ears or parts of their ears. This may allow the headgear to fit a broader range of people without causing interference with their ears and may provide added comfort as the headgear may be adjusted by the patient into a greater range of positions without contacting their ears.

As seen in FIG. 58 the headgear is shaped to provide extra clearance from the patient's ears than the headgear of FIG. 57, particularly in a region above and behind the patient's ears.

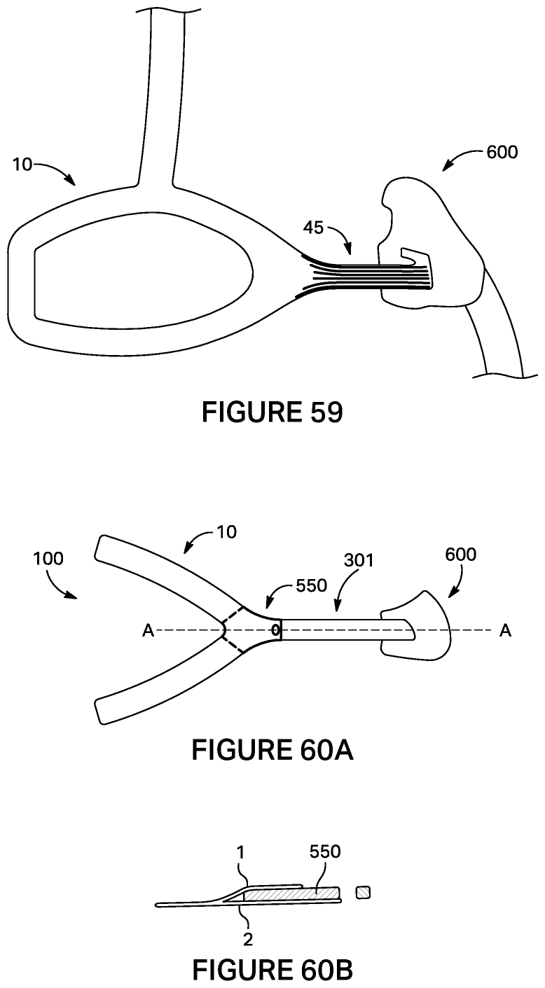

The use of fused structures may enable a reduction in the number of straps used in connecting a headgear 10 to a patient interface 600. For example, FIG. 59 shows a side view of a headgear 10 which connects to an interface 600 by a single point. The headgear may include parts that sit on both sides of the patients, head, but the use of a highly stiffened fused structure 45 may facilitate only a single point of connection between the headgear 10 and the interface 600.

Such a fused structure 45 may be formed by a highly fused panel, and particularly a textile or fabric panel. They may additionally or alternatively be formed by a rigid material that is fused to an underlying panel or panels.

In addition to being used for treating or joining together panels to form a headgear, fusing may be used to join other fixtures to the headgear. This may include where a fused structure is to be provided by a rigid material that is fused to the underlying panel or panels. It may also include a where attachment fixtures such as clips, buckles, or hook and loop pads are to be included as part of the headgear.

FIG. 60A shows a side view of a headgear 10 having a rear portion 100 and a single strap 301 and 302 on each side of the headgear. The headgear 10 is connected to an interface 600. A fixture 550 has been fused to panels of the rear portion 100.

The fixture 550 includes a slot through which the side straps can pass to be folded back onto and secured to themselves.

FIG. 60B shows a partial section view through the line AA of FIG. 60A, showing the fixture 550 and the slot to allow the strap to be passed through it. The rear portion 100 of the headgear at the fixture 550 includes a first panel 1 and a second panel 2 which are lapped either side of the fixture 550 and secured to it.

The securing of such fixtures may be by fusing. The fusing process may be the same or different to that used to fuse the headgear panels. The fixtures may be secured at the same time or in a separate step to the fusing of the headgear panels.

For example, where the headgear panels are fused by a high frequency welding, the fixture 550 of FIGS. 60A-B may be of a dipolar material and may be fused to the first panel and second panel 2 at the same high frequency welding step that the panels are fused together. The fixture 550 may alternatively be fused to the panels 1 and 2 in a subsequent high frequency welding step.

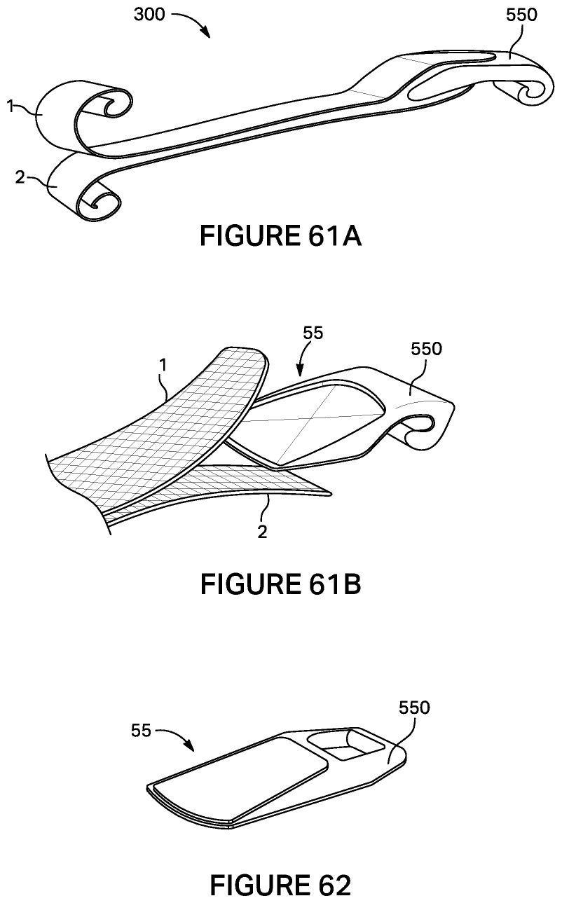

FIG. 61A shows part of a headgear, strap 300, which has a fixture 550 fused to it. The panels 1 and 2 of the strap 300 overlap and underlap the fixture 550 and are be fused to provide a seamless transition between the strap and the fixture.

As illustrated in FIG. 61B, showing the arrangement of FIG. 61A before the panels 1 and 2 are fused to the fixture 550, the fixture 550 has recesses 55 formed in it which correspond to the shape of the ends of the panels 1 and 2. The height of the fixture 550 also tapers towards the strap. These features may aid in providing a seamless transition between the strap and the fixture.

FIG. 62 shows an example of another form of a fixture 550 which provides a slot, rather than the hook feature illustrated in the fixture of FIGS. 61A and 61B.

While generally described as being connected by fusing, in other configurations fixtures may be attached to the panels of the headgear by other methods, such as by an adhesive or by stitching, for example.

While fixtures as separate parts may be attached to a panel or panels of the headgear by fusing, such as is illustrated and described in relation to FIGS. 61A-B and 62, fixtures may additionally or alternatively be provided to a headgear by other methods.

A panel or panels may be formed into a desired shape then fused to form that part of the panels into a solid plastic. For example, the end of a panel or panels may be curled to form the shape of the clip fixture 550 of FIG. 61A, then fused to solidify the panel or panels in that shape.

While a panel or panels themselves may be fused to form a fixture, the fused parts may instead be used to form a substrate for overmolding, where the overmolding forms the desired fixture.

For example, an end of a panel or panels may be fused to make a part that is sufficiently rigid to allow overmolding. In such a configuration the end of the panel or panels may be formed into the or part of the shape of the final fixture or may be fused in a flat configuration.

Once imparted with sufficient structural integrity from fusing, the end of the panel or panels may be inserted into a mould and overmolded with another material, for example a solid plastics material.

It may be desirable to ensure that edges of the headgear, or edges of individual panels, are smooth and soft to maximise comfort for the patient. Where an edge of the headgear is defined by a cut edge of a panel, unless the panel is made of a non-unravelable material, the edge may require treatment such as fusing to prevent it unravelling. Such treatments may stiffen or harden the edge and make it less comfortable. Even if edge is defined by a non-unravelable material, it may be desirable to further soften or smooth the edge.

One method of treating the edges of the headgear or of individual panels is to fold another panel about the so that the periphery of the headgear or panel is no longer defined by a cut edge but the continuous rolled surface of a panel.

FIG. 63 illustrates a first panel 1 which has had a second panel 2 folded over one of its edges. The panels may then be fused together, either about only a portion away from the folded edge or across the whole of the panels 1 and 2.

FIG. 64 shows another configuration of a first panel 1 with a second panel 2 folded about one edge of it. In the configuration of FIG. 64 the folded panel is not folded hard onto itself, and instead defines a void 530 at the edge. Such a void may be formed by using a sufficiently stiff panel for the second panel 2, such that it does not fold fully back on itself. The void may also be formed by the either temporary or permanent use of an insert which is located at the edge and the second panel 2 is folded about it.

FIG. 65 shows another configuration of a first panel 1 with a second panel 2 folded about both its lateral edges. In FIG. 65 the second panel 2 also defines voids 535 and 536 at each of the folded edges.

FIG. 66 shows the configuration of FIG. 65, but where the voids 535 and 536 are filled with respective edge members 532 and 533. The edge members 532 and 533 may be provided at the edges of the first panel 1 when the second panel 2 is wrapped around the first panel 1, or where the voids are formed without need of inserts, the edge members 532 and 533 may be inserted into the voids 535 and 536 after the second panel 2 is wrapped about the first panel and they are fused together.

Edge members may act to hold a void open. They may additionally or alternatively also provide structure, such as a shape memory to the edge. For example, where a metal filament is used as an edge member, it may be deformable but aid in holding the edge in a set shape. Further examples of an edge member may include a bead or rope piping.

FIG. 67 shows another second panel 2 wrapped about both edges of a first panel 1, but so that one face of the first panel 1 is left substantially exposed. The first and second panels may then be fused at the location of the arrows 817. Such a configuration may be desirable where the surface properties, for example a UBL property, of the first panel is to be utilised but the edges of the panel are to be softened or rounded.

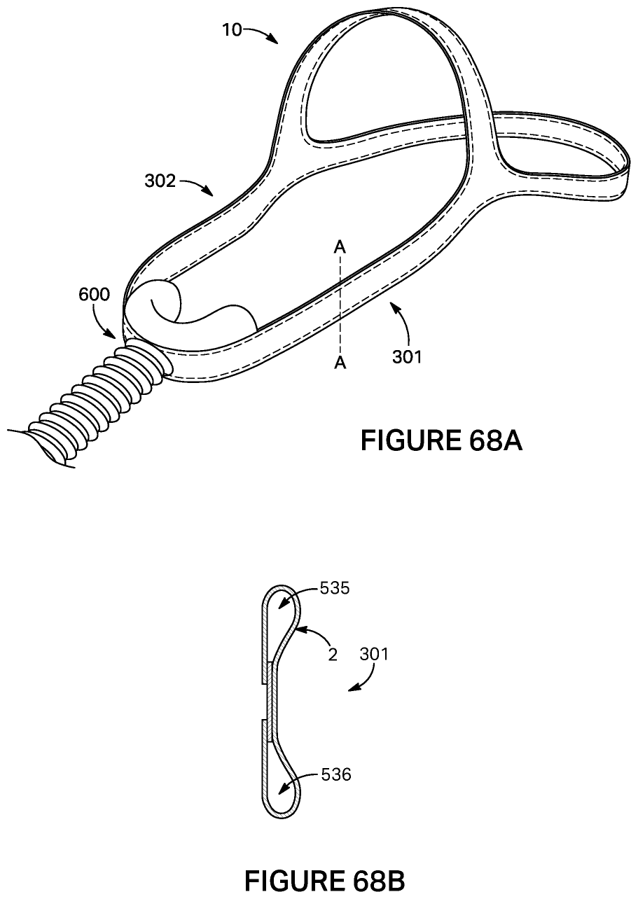

FIG. 68A shows a headgear 10 according to one embodiment. The headgear 10 is attached to an interface 600. The headgear has two straps 301 and 302, and a rear portion 100. The headgear is formed from a plurality of lapped and fused panels, and at least some of the edges of the headgear are defined by folded panels rather than cut edges.

FIG. 68B shows a cross-section through the line A-A of FIG. 68A. The headgear strap 301 at the cross-section has a first panel 1 which is surrounded by a wider second panel 2 which is folded about the first panel. The folded edges are each approximately the same width as the first panel 1, so the assembly of the first and second panels is in total approximately three times the width of the first panel 1.

Each of the folded edges define voids 535 and 536 where the second panel 2 folds back on itself.

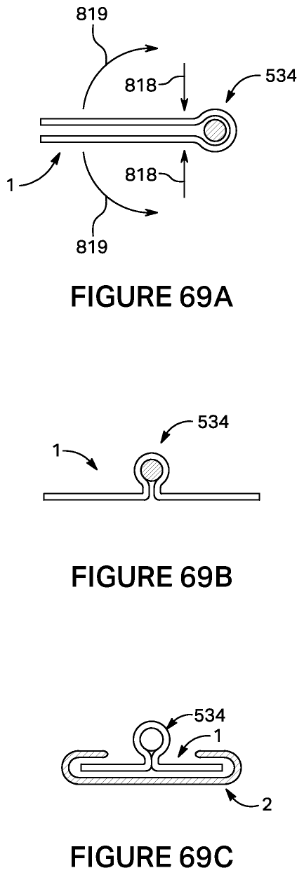

The use of an insert or piping about which a panel is folded and welded may be utilised to define other features in addition to edges of the headgear. For example, as seen in FIG. 69A a first panel 1 may be folded about a bead 534 and then the panel fused to itself adjacent to the bead 534 at the location of the arrows 818. The free ends of the first panel 1 may then be folded outwards in the direction of the arrows 819, to the configuration shown in FIG. 69B. In this configuration the bead and second panel wrapped about it may define a slider, along which and adjustable fixture such as a hook or buckle may be slid.

As illustrated in FIG. 69C the assembly of FIG. 69B may further be wrapped by a second panel 2 provided folded rather than cut edges to the assembly.

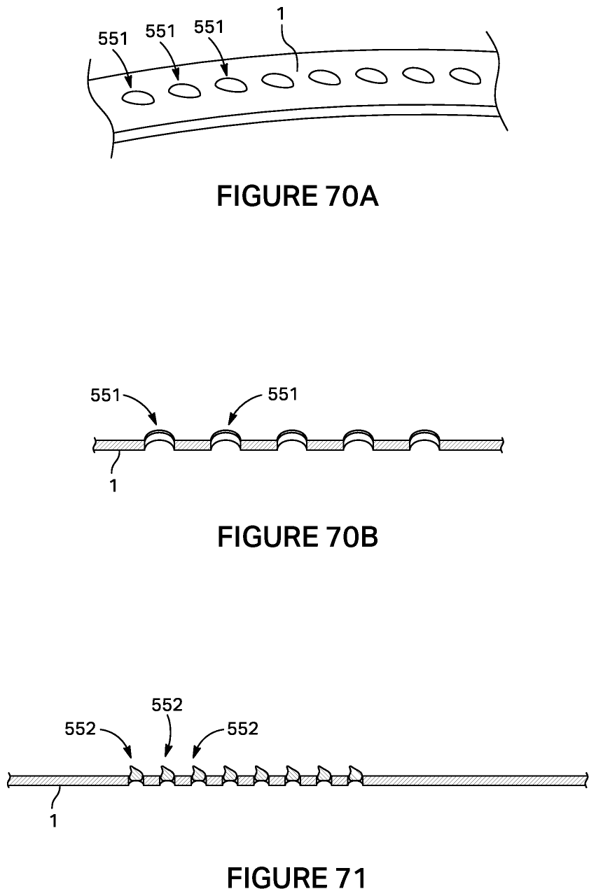

Fusing may be deployed to form other types of adjustment mechanisms. For example, FIG. 70A illustrates a panel or layup of panels which have been fused to form an array of rigid plasticised buttons 551. The buttons 551 are localised bumps in the panel or layup of panel, which may mate with another panel or fixture having a corresponding array of holes into which the buttons 551 may couple, in the same manner as the snap-fit adjustment of a baseball hat.

FIG. 70B shows a cross-section along the array of buttons 551 of FIG. 70A, illustrating how the panel or panels may be fused into rigid bumps.

FIG. 71 shows another form of adjustment features, where a panel or layup of panels have been fused to form an array of teeth 552. The teeth 552 may be used to provide a one-way adjustment mechanism in the nature of a zip-tie.

Adjustment features may be formed by fusing causing a melting and drooping of the material to form the buttons. They may also be formed using dies, such as the dies 503 and 504 of FIGS. 9A and 9B, which have projections and depressions corresponding to the desired shape of the adjustment features.

Additional materials may be added between panels of the headgear before the panels are fused together. These materials may be fusible or non-fusible materials. For example, a void or pocket forming insert may be placed between the panels, or an edge forming bead or filament may be placed between panels or within a fold of one panel.

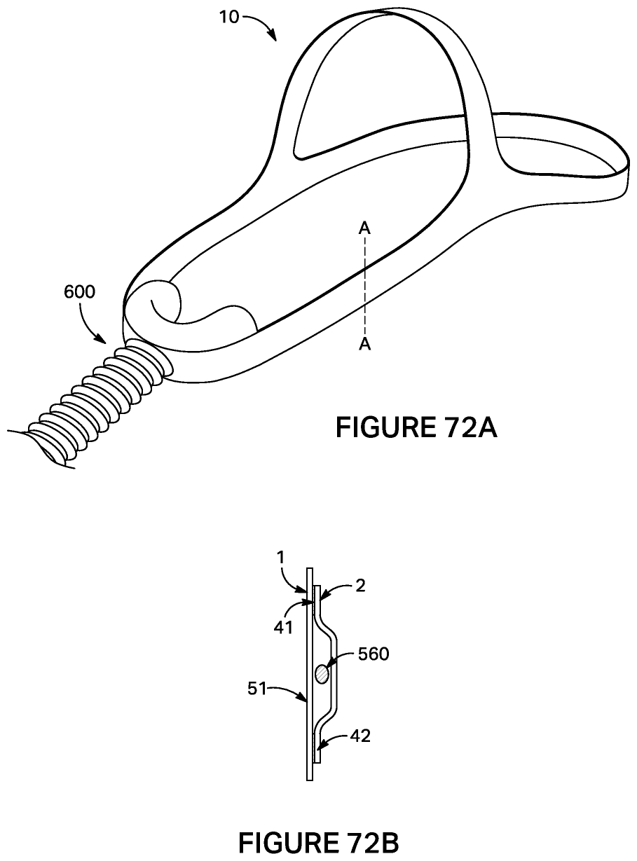

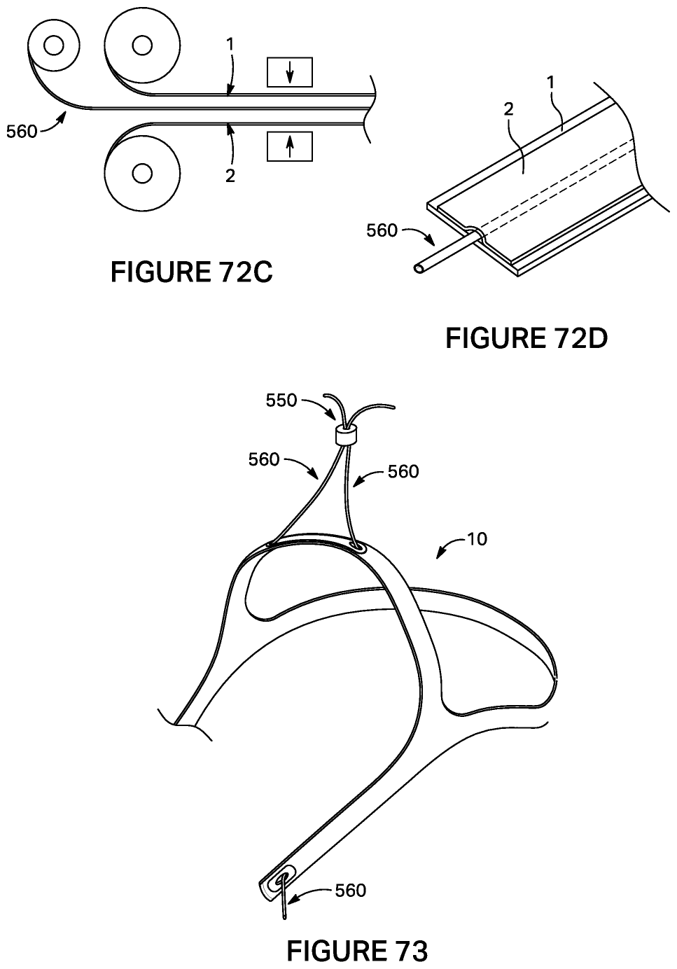

FIG. 72A shows an example embodiment of a headgear 10 attached to an interface 600.

FIG. 72B is a cross-section through the headgear 10 at the line A-A of FIG. 72A. At the cross-section the headgear 10 has a first panel 1 which is partially lapped by a second panel 2. Peripheral portions of the lapped panels are fused to define a first fused zone 41 and second fused zone 42. A filament 560 is located between the two lapped panels, and within a non-fused zone 51 between the two fused zones 41 and 42.

The filament 560 may provide additional structure to the headgear, such as to provide shape memory where the filament is a plastically deformable metal.

In other forms, such a filament 560 may be used for providing fit adjustment to the headgear, as will subsequently be described.

The filament 560 may be inserted placed between two panels before they are fused in a discrete fusing process. In other forms, such as illustrated in FIG. 72C, the filament 560 may be placed between a first panel 1 and second panel 2 as part of a continuous fusing process.

FIG. 72D is another view of a fused first panel 1 and second panel 2 with a filament 560 provided within a pocket defined by a zone where the two panels are not fused to each other.

FIG. 73 is a partial view of a headgear 10 with a filament 560 in each of the side straps 301 and 302 which extends along the top strap 200. By securing either the side strap-ends or top strap-ends of the filaments 560 then drawing on the other ends the fit of the headgear may be adjusted in the same manner as a drawstring.

As illustrated in FIG. 73, the headgear 10 may include a fixture 550 that the filaments 560 pass through to enable fixed adjustment of the tension on the filaments 560.

According to the disclosure, a headgear may include a rear portion and two or more straps for connecting between the rear portion and a patient interface. Some or all of the straps of a headgear may be formed integrally with the headgear, by being formed from one or more of the same lapped panels as the rear portion of the headgear. In other configurations, one or more of the straps may be formed separately, then attached to the rear portion of the headgear. As previously described, FIG. 11 illustrates a configuration where separate straps 200 and 301-304 are attached to a rear portion 100 to form the headgear 10.

One or more straps of the headgear may have either a straight or non-straight shape when laid flat. The use of straps having a straight shape when laid flat may increase the yield of the material or materials from which the straps are formed.

The straps of the headgear may be made from a single panel or multiple lapped panels like the rear portion. Or, one or more of the straps of the headgear may be of another material or materials, for example a foam and fabric laminate.

Straps which are formed separately to the rear portion of a headgear may be attached to the rear portion by one or more different methods. In at least some configurations, one or more headgear straps may be fused to the rear portion. The fusing may be by a welding, such as for example an ultrasonic welding.

In other configurations, one or more straps may be additionally or alternatively attached to the rear portion by other methods, such as gluing or stitching.

Instead of being permanently attached, some straps may be removably attached to the rear portion of the headgear. This may facilitate, for example, the replacement of worn straps, or changing the type or size of the straps used with a particular rear portion. One or more straps may be removably attachable to the rear portion by, for example, fasteners or clips, or a hook and loop arrangement.

Where the attachment of one or more straps includes the welding of the strap and rear portion together, one side of the strap may be welded to a panel or panels of the rear portion. In the configuration of FIG. 11, for example, the straps 200 and 301-304 have each been laid on top of the lapped first and second panels 1 and 2 and attached to them at only one side of each respective strap.

In other configurations, a strap may be at least partially sandwiched between two panels of the rear portion. FIG. 17A illustrates a rear portion 100 which has the non-fused zones 51-54 of the first panel 1 and second panel 2 at each of the strap connection portions 120, such that part of a strap may be located between the first panel 1 and second panel 2 and the components attached to each other, such as by welding. FIG. 17B then illustrates respective straps which have been welded between the first panel 1 and second panel 2 at each of the strap connection portions 120.

The strength of a fused connection between a strap and rear portion may depend on the type of strap utilised and its material properties. For example, for some types of fabric laminated foams which may be used as straps, a fused or more particularly welded connection between the laminated foam and a rear portion may be relatively enhanced where the laminated foam is welded to a non-fused part of the rear portion.

More particularly, the connection may be relatively enhanced where the laminated foam is welded to a single panel which has not been fused, rather than two or more lapped panels which are unfused.

FIG. 74 shows a headgear 10 having a rear portion 100 with a first panel 1 and second panel 2. At the lateral extents of the rear portion, the first panel 1 extends beyond the second panel 2 to define strap connecting portions 120a and 120b of the first panel 1. The straps 200a/301 and 200b/303 have been lapped against the strap connection portions 120a and 120b and fused together with the first panel 1.

As illustrated in FIG. 74 the straps 200a/301 and 200b/303 have been lapped against a patient-facing side of the first panel 1, being the side opposite to the second panel 2. In other configurations the straps 200a/301 and 200b/303 may be lapped against the non-patient facing side of the first panel,

While the straps 200a/301 and 200b/303 at the strap connection portions 120a and 120b are illustrated in FIG. 74 as only partially lapped with the first panel 1, in other configurations the unlapped part of the first panel 1 at the strap connection portions 120a and 120b may be of sufficient size to allow the full width of the straps to be lapped against it.

The attachment of a strap by fusing with an unlapped part of the first panel 1 may be used at any one or more of the straps of a headgear, including any lower side straps 302 and 304 of a headgear.

As previously described, it may be desirable to limit curves and changes in width of headgear straps, in order to increase the yield of the material or materials from which the straps are formed. FIG. 75 illustrates a configuration where the headgear 10 includes separate straps 200a, 200b, and 301-304.

Except for the strap end feature 330 of the second top strap 200b, each of the straps 200a, 200b, and 301-304 of the headgear 10 of FIG. 75 have a constant width along their respective lengths.

While the second top strap 200b is illustrated with a widened end having a slot to receive the end of the first top strap 200a, it will be appreciated that other forms of attachment between the top straps, such as a buckle or hook and loop fastening system, may be used to allow the second top strap 200b to have a constant width along its entire length.

As illustrated in FIG. 75, each of the straps 200a, 200b, 302 and 304 are also straight along their entire respective lengths. The upper side straps 301 and 303 are also straight, except for a single curve towards the rear portion 100 attached ends. These curves may aid in locating the strap ends in desired proximity to strap connection fixtures of a patient interface in use.

In other configurations however the upper side straps 301 and 303 may be entirely straight along their length.

As illustrated in FIG. 75, the straps 200a, 200b, and 301-304 have been placed on top of the lapped first and second panels and are fused to the second panel 2 at the lapped region 21 of the rear portion 100. This provides a headgear 10 where the straps 200a, 200b, and 301-304 are located wholly at the non-patient facing side of the headgear.

While illustrated in FIG. 75 as being fused only to the second panel 2 and not to the non-lapped portion 31 of the first panel 1, in some configurations the straps may be fused to the parts of both the second panel 2 and first panel 1 which they are lapped against.

As previously described, for example in relation to FIGS. 48A-D, the connection of a strap to a rear portion of a headgear may be facilitated by the insertion of a strap end into a pocket of the rear portion.

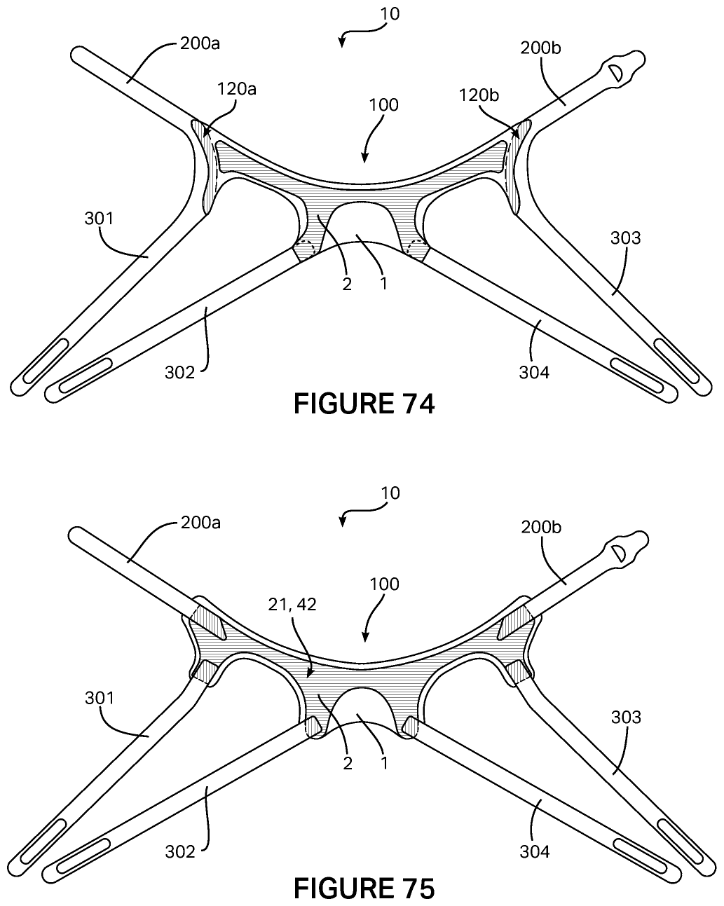

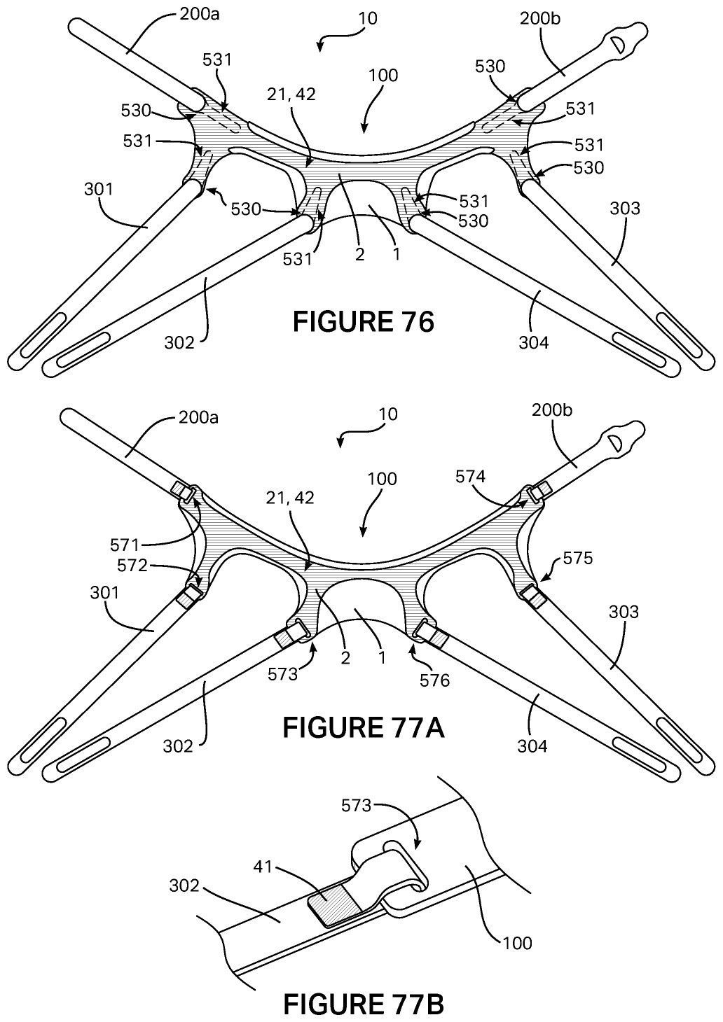

FIG. 76 illustrates a further embodiment of a headgear 10 having a first and second top straps 200a and 200b, and two pairs of upper and lower side straps 301-302 and 303-304. Each of the straps are connected to the rear portion 100 by the insertion of each strap end into a respective cut-out 530 of the second panel 2.

Where the first panel 1 and second panel 2 are fused together before connection of the straps, the fusing may leave unfused areas adjacent each of the cut-outs 530 to define pockets 531 to accommodate the ends of each of the straps, as described in relation to FIGS. 48A-D. Once inserted into the pockets, the strap ends and first and second panels can then be fused together.

Where the first panel 1 and second panel 2 are fused together at the same time as fusing the straps to them, no predefined pockets between the first and second panels are needed as the strap end can simply be inserted between the unfused first and second panels.

As seen in FIG. 76 each of the straps 200a, 200b, and 301-304 are of continuous widths and straight, excepting the ends of the side straps 301-304 in the pockets of the rear portion 100, and the strap connection fixture of the second strap 200b. While the strap ends of the side straps 301-304 are shown angled relative to the remainder of each side strap, in other configurations the strap ends connected to the headgear may not be angled relative to the remainder of each respective strap.

FIG. 77A illustrates another form of strap connection to a rear portion 100. As seen in FIG. 77A, end portions of individual straps 200a, 200b, and 301-304 each pass through respective slots 571-576 of the strap connection portions of the rear portion 100. The strap ends are then folded back and connected to themselves. The connection may of a releasable form, using for example a hook and loop fastening system.

Or, as illustrated in FIG. 77A and in further detail in FIG. 77B, when inserted through the slots of the rear portion the straps may be fused to themselves as illustrated by the fused zone 41. The fusing of a strap to itself may be preferred where the straps are made of a fabric laminated foam, which may fuse more effectively to another fabric laminated foam than to a textile alone, such as the first panel 1 or second panel 2.

The slots 571-574 may be slots through the thickness of the rear portion 100, for example through both a first panel 1 and second panel 2.

In other forms, for example where it is desirable to locate the straps away from the patient contacting side of the rear portion, the slots 571-574 may pass through only one or more of the outer panels of the rear portion. In this configuration the strap ends would enter a pocket between the first panel 1 and second panel 2 before extending through a respective slot and being folded back onto themselves.

While in FIG. 77A slots are formed in a panel or panels of the rear portion 100 to accommodate the attachment of straps, in other forms some of the slots may be formed in a strap, to allow for the connection of other straps to it.

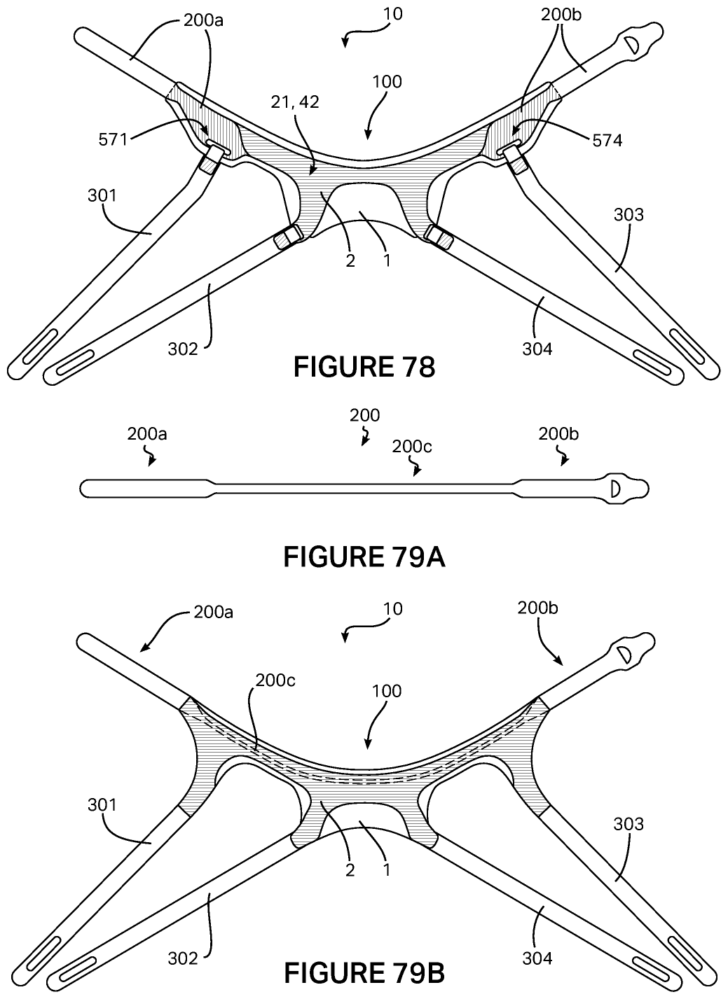

For example, in FIG. 78 the first top strap 200a and second top strap 200b have slots 571 and 574 formed in them, which accommodate the upper side straps 301 and 303.

In the configuration of FIG. 78 the top straps 200a and 200b each lap the rear portion up to a most lateral extent of the ear loops 320.

The slots 571 and 574 may be slots only through the top straps 200a and 200b, or may alternatively be slots through the top straps 200a or 200b and one or more of the panels of the rear portion 100 to which they are lapped.

The portions 200a and 200b of a top strap may be provided as part of two of the side straps, for example as illustrated in FIG. 74. In other forms they may be provided as separate parts, such as illustrated in FIG. 75. In still other forms, the portions 200a and 200b of a top strap may include one continuous piece, for example as previously illustrated and described in relation to FIG. 34. A top strap which includes a continuous piece of material may enhance the ability for loads to be transferred between the ends of the top strap, without total reliance on the material of the rear portion 10 for transferring those loads. The use of a top strap which includes a continuous piece of material also removes the need for separate top strap attachment fixtures on the rear portion.

FIG. 79A shows a top strap 200 which has two top strap ends 200a and 200b, with a relatively thinned central portion 200c connecting therebetween. The top strap 200 has a straight shape. Its non-curved formed shape may allow for a relatively improved yield of the substrate or substrates from which it is formed.

FIG. 79B sows the top strap 200 of FIG. 79A incorporated as part of a headgear 10, sandwiched between the first panel 1 and second panel 2 and fused thereto. Because the central portion 200c of the top strap 200 is relatively thin, it may be able to be shaped to follow the curve of the upper edge of the rear portion with minimal out of plane warping or distortion. By this configuration, a headgear can be provided with a continuous and curved top strap 200 and an increased material yield, relative to a top strap 200 which is itself formed in the desired curved shape.

The top strap 200 may formed from a fabric laminated foam.

Where the properties of the straps and rear portion of a headgear mean that the straps may more securely be fused onto their own material than to the panels of the rear portion, the rear portion may have portions of the same material as the straps added to it to act as strap connection portions.

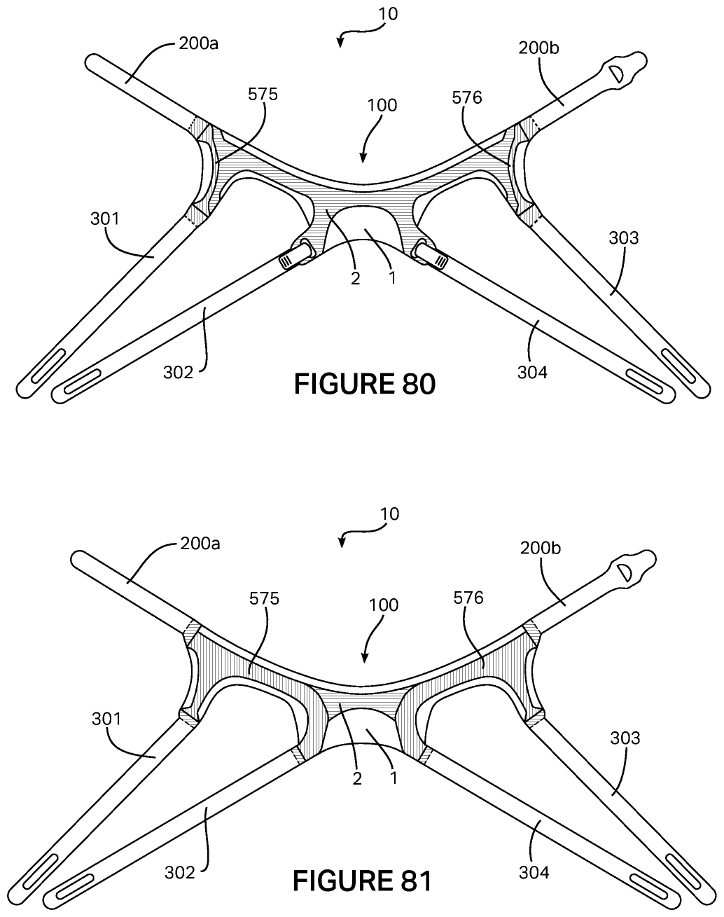

FIG. 80 illustrates a headgear 10 where two wishbone-shaped strap material portions 575 and 576 have been lapped to the second panel 2 at each lateral extent of the rear portion 100. For example, where the straps are made from a fabric laminated foam, the strap material portions 575 and 576 may also be a fabric laminated foam.

The straps 200a and 200b, and upper side straps 301 and 303, have each been laid on top of the strap material portions 575 and 576, and fused thereto.

The strap material portions 575 and 576 may be fused to the rear portion 100. Alternatively, particularly where a fused connection between the two materials may not provide sufficient strength, the strap material portions 575 and 576 may be fixed to the rear portion 100 by other methods such as by an adhesive or by stitching.

The thinned wishbone-like configuration of the strap material portions 575 and 576 may mean they can be cut in a straight shape, then curved when they are applied to the rear portion without causing undue distortion.

While illustrated in FIG. 80 as a single piece at each side of the rear portion 100, strap material portions may be provided separately on the rear portion for one or more of the straps of a headgear.

While the strap material portions may be a fabric laminated foam, particularly where straps are of a fabric laminated foam, the strap material portions may alternatively be an adhesive, such as a hot melt adhesive.

FIG. 81 illustrates another example of a headgear 10 incorporating strap material portions 575 and 576 to aid in the connection of straps 200a, 200b, and 301-304 to the rear portion 100. As seen in FIG. 81, the strap material portions 575 and 576 each continuously extend between the three strap connection points of each lateral side of the rear portion.

As well as being bonded to each other, a strap and rear portion of a headgear can be connected indirectly using an intervening part.

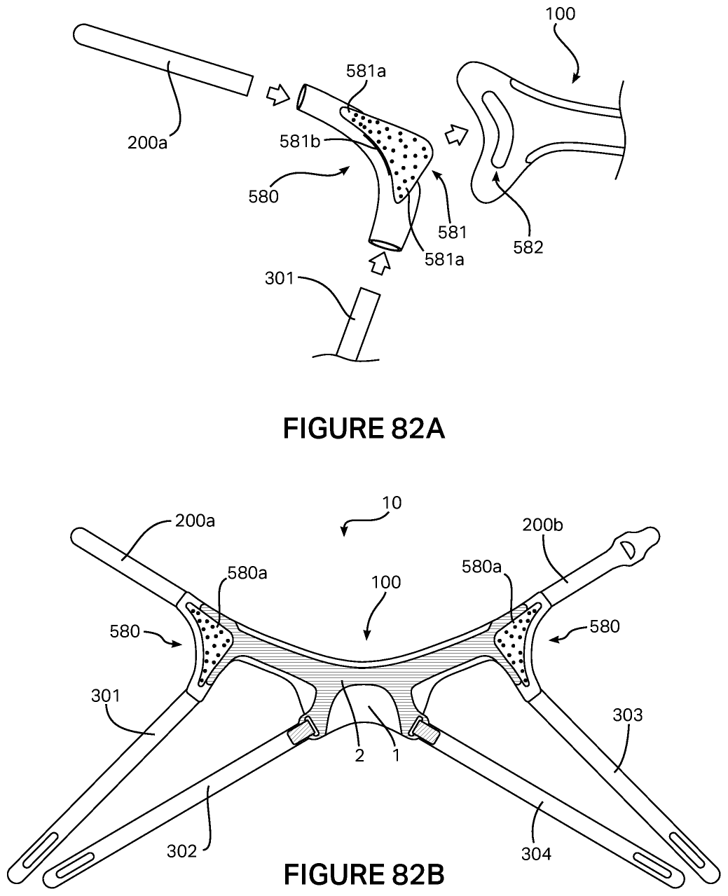

For example, as illustrated in FIG. 82A, two straps 200a and 301 may be inserted into an overmold piece 580, which is then connected to the rear portion 100 of the headgear. In FIG. 82A a mounting post 581 depends from the overmold piece 580. The mounting post 581 has a barbed or mushroom-like shape, with a base 581b that connects to the overmold piece 580 and a head with laterally extending wing portions 581a. As seen in FIG. 82A the rear portion 100 has a cut-out 582. The length of the cut-out 582 is less than the length of the mounting post 581 between its two lateral wing portions 581a. The rear portion 100 and strap assembly are connected by passing the cut-out 582 over the mounting post 581 so that it is retained by the wing portions 581a.

By this configuration a strap assembly may be removably connected to a rear portion of a headgear. This may allow a straps or sets of straps, or the rear portion to be changed—for example to replace a worn-out item, or to provide a different size-without requiring the entire replacement of the headgear.

A headgear 10 with respective overmold pieces 580 overmolded on straps 200a and 301, and 200b and 303, and connected to the rear portion 100 by the mounting posts 581, is illustrated in FIG. 82B.

The rear portion 100 is illustrated in FIGS. 82A and 82B as connecting to the strap assembly by the mounting post 581, the rear portion may also be overmolded to form a permanent rather than a removable connection.

As previously described, it may be desirable to locate the connection points of one or more straps of the headgear away from the patient-facing side of the rear portion. This may improve a continuousness or smoothness of the patient-facing side of the headgear, and so improve the comfort of the headgear.

It may also be desirable however to connect both sides of a strap to the rear portion, rather than only having a connection at one side of the strap. Connecting both sides of the strap to the rear portion may provide an increased strength of join. It may also reduce any torsion of the headgear at the join.

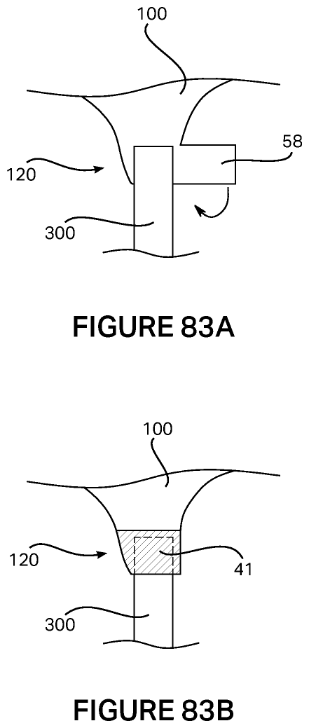

FIG. 83A illustrates a strap connection portion 120 of a rear portion 100. At the strap connection portion 120 the rear portion 100 includes an extension 58. A strap 300 is lapped on one side against the rear portion 100 at the strap connection portion 120, and the extension 58 is folded over the strap 300 so that it is lapped on both sides by the rear portion 100.

As illustrated in FIG. 83B, the strap and rear portion may be fused together at the strap connection portion 120 to attach the strap 300 with the rear portion 100.

While various preferred embodiments utilise fusing to treat a non-lapped region or to join panels at a lapped region, other methods of joining or treating panels may also be utilised. For example, panels may be joined by the use of an adhesive, or various panels or panel parts may include stitched joins, particularly at locations where such stitching may not undesirably impact on the bulk of the headgear or particularly thickness of the joins and comfort of the patient when wearing the headgear. Panels may further be joined by the use of fixtures to hold the panels together.

An adhesive may be applied between panels to be joined, or it may additionally or alternatively be applied to the lapped panels such as by dipping or soaking the panels in an adhesive.

The term adhesive will be understood to encompass any one or more commonly available types or combinations of adhesives. The adhesive may for example be an acrylic adhesive, an anaerobic adhesive, or a cyanoacrylate adhesive.

The adhesive may for example be an ester or ether-based compound. More specifically it may comprise nylon-polyamide, be nylon-polyamide, ester-polyurethane, polyester, or polyolefin.

Particularly where the adhesive is provided as a solid or semi-solid, but potentially also where it is a liquid or a gel, the adhesive may need to be activated to initiate or accelerate the process of curing the adhesive to form the join between the lapped panels. For example, in the case of a solid or semi-solid adhesive the adhesive may be activated by melting from its solid or semi-solid state. In other forms the adhesive may be activated by the combination of two components of the adhesive, such as in the case of a two-component acrylic adhesive.

While the process of joining panels by forming a bond by an adhesive will herein be referred to as a curing of the adhesive, it will be understood that such curing may include the forming of bonds by one or more of a chemical reaction within the adhesive or combination of adhesives, by the presence of a particular environment (such as anaerobic environment) or contact with a particular type of material (such as an alkaline material), by drying, or by an application of one or more of pressure, heat, sound, or light, or any other commonly utilised method to transform the adhesive into a bonded state.

In an example, the adhesive may be in the form of a solid or semi-solid hot-melt adhesive. Heat may be applied to activate the adhesive by melting it, and the adhesive may cure such as by one of the above-referenced curing processes in order to form the join between the respective lapped panels.

An adhesive may be provided as a separate layer and assembled between the respective panels, where it may then be activated to create the bond. Particularly where the adhesive is a liquid or gel adhesive, adhesive may be applied to a region of one or both of the lapped panels during the assembly of the panels.

Where the adhesive is in the form of a hot-melt adhesive, it may be provided as a tape. This tape or sheet of adhesive may be able to be cut to form desired plane shapes to provide the desired bonded regions of panels.

Such a tape or sheet hot-melt adhesive may additionally comprise a sticky or adhesive surface or surface coating, such that the adhesive may provide at least some provisional hold of the panels to which it is assembled before the adhesive is activated.

While according to various embodiments adhesive may be used alongside a fusing of the panels to join and/or treat parts of the headgear, at least some preferred embodiments may exclude the use of an adhesive. The use of an adhesive- or any other joining method which is additive-will inherently result in an increase in one or both of the weight or thickness of the joined panels.

For example, where an adhesive tape is used it will be laid between the panels which are joined. While the adhesive may be melted fully or partially into one or both of the panels, the adhesive will add weight to the joined panels relative to a fusing of the panels directly to each other.

While generally illustrated as being fully lapped by the first layer, according to various embodiments the second layer may only be partially lapped by the first layer.

While generally illustrated as being fused together across whole parts of the lapped region, the lapped panels of a headgear may be fused together only or primarily about a border of the lapped region. In such situations the boundary where fusing is applied may however still extend beyond the lapped region onto any adjacent non-lapped region.

According to various aspects of the disclosure a headgear comprising a plurality of panels each of which is at least partially lapped with another of the panels and which are fused together may have a reduced weight relative to a headgear formed by other methods.

A headgear according to the disclosure may have a weight of less than about 30 g, of less than about 20 g, or of less than about 10 g.

In particular, various embodiments of a headgear 10 according to the disclosure may have a weight of about 17.5 g to about 27.5 g, and more particularly of about 25 g.

While the foregoing description has made reference to various general concepts of lapping panels together and various features of particular embodiments of headgear formed by such lapped panels, it is intended within the scope of the disclosure that any of the general concepts or features of particular aspects or embodiments may be combined with each other in any number of different ways to provide a headgear or part thereof.

1-21. (canceled)

22. A rear portion for a headgear for a patient interface, the rear portion comprising:
a first textile panel; and
a second textile panel, the first textile panel and the second textile panel defining a lapped region in which the first textile panel and the second textile panel respectively overlap and underlap each other and a non-lapped region in which the first textile panel is not lapped,
wherein at the lapped region, adjacent surfaces of the first textile panel and the second textile panel are fused together.

23. The rear portion of claim 22, wherein the adjacent surfaces of the first textile panel and the second textile panel at the lapped region are fused directly to each other without any interposed material.

24. The rear portion of claim 22, wherein the lapped region comprises a fused portion and a non-fused portion.

25. The rear portion of claim 22, wherein the lapped region comprises a transition zone between a fused portion and a non-fused portion, the transition zone defining a degree of fusion between that of the non-fused portion and the fused portion, and the transition zone comprises a gradient of degrees of fusion between that of the fused portion and the non-fused portion.

26. The rear portion of claim 22, wherein the rear portion comprises different stretch properties at each of:
(a) a fused portion of the lapped region,
(b) a non-fused portion of the lapped region, and
(c) the non-lapped region of the first textile panel.

27. The rear portion of claim 22, wherein adjacent surfaces of the first textile panel and the second textile panel about a periphery of the lapped region are fused together.

28. The rear portion of claim 22, wherein the second textile panel is fully lapped by the first textile panel, and the adjacent surfaces of the first textile panel and the second textile panel are fused together about a whole periphery of the second textile panel.

29. The rear portion of claim 28, wherein a periphery of the lapped region includes a border both sides of a perimeter of the lapped region.

30. The rear portion of claim 29, wherein the first textile panel and the second textile panel at the lapped region are continuously fused together across the lapped region between two or more points about the perimeter of the lapped region.

31. The rear portion of claim 22, wherein the first textile panel and the second textile panel are fused together at a substantial entirety of the lapped region.

32. The rear portion of claim 22, wherein the non-lapped region is provided at a lower periphery of the rear portion along one or more curved portions of the lapped region.

33. The rear portion of claim 32, wherein the non-lapped region at the lower periphery of the rear portion comprises a varying width.

34. The rear portion of claim 32, wherein the non-lapped region at the lower periphery of the rear portion comprises an edge with a radius of curvature less than that of an adjacent curved portion of the lapped region.

35. The rear portion of claim 34, wherein the non-lapped region at the lower periphery of the rear portion defines one or more crescent shapes.

36. The rear portion of claim 22, wherein the rear portion comprises a central section comprising laterally spaced strap connection portions, and wherein the non-lapped region is a first non-lapped region and is located between the laterally spaced strap connection portions of the central section.

37. The rear portion of claim 36, wherein the rear portion comprises lateral sections either side of the central section, each lateral section comprising a strap connection portion at a lateral extent thereof, and wherein the rear portion comprises a second non-lapped region located at a lateral peripheral edge of the rear portion between each strap connection portion of the lateral sections and a respective strap connection portion of the central section.
